(19) United States
(12) Patent Application Publication
(10) Pub. No.: US 2011/0092517 A1
(43) Pub. Date: Apr. 21, 2011

(54) Use of PPAR Delta Ligands for the Treatment or Prevention of Inflammation or Energy Metabolism/Production Related Diseases

(75) Inventors: Ronald Barbaras, Seilh (FR); Daniela Carmen Oniciu, Balma (FR); Jean-Louis H. Dasseux, Toulouse (FR); Robert A. Scott, Rouffiac-Tolosan (FR); John R. Wetterau, Indianapolis, IN (US)

(73) Assignee: Cerenis Therapeutics S.A. & Nippon Chemiphar Co., Ltd.

(21) Appl. No.: 12/856,532

(22) Filed: Aug. 13, 2010

Related U.S. Application Data
(60) Provisional application No. 61/234,231, filed on Aug. 14, 2009.
(60) Provisional application No. 61/251,655, filed on Oct. 14, 2009.

(51) Int. Cl. A61K 31/496; A61P 11/00; A61P 11/06; A61P 29/00; A61P 9/10; A61P 19/02; A61P 3/10; A61P 3/04; A61P 5/50; A61P 9/12; A61P 3/06; A61P 21/00; A61P 25/08; A61P 17/14; A61K 31/426; A61K 31/423; A61P 17/02; A61K 31/421; A61K 31/4164; A61K 31/4709; A61K 31/506; A61K 31/4439; A61K 31/415; A61K 31/381

(52) U.S. Cl. 514/254.02; 514/365; 514/375; 514/374; 514/398; 514/314; 514/256; 514/342; 514/406; 514/438; 514/443

(57) ABSTRACT

Provided herein are methods for treatment, prevention, or amelioration of one or more symptoms of a disease or condition related to disorders of insulin and/or glucose metabolism, inflammatory conditions, mitochondrial disease, muscle disorders, or pulmonary disorders, involving administering a PPARδ agonist or a pharmaceutical composition comprising a PPARδ agonist. In one embodiment, the disease or condition is selected from myopathy, inflammatory vascular diseases, Parkinson's and Alzheimer's diseases, systemic inflammatory disorders, renal ischemia, inflammatory rheumatic disorders, and inflammatory diseases of the lung. In another embodiment, methods for increasing oxidative muscle fibers, reducing mitochondria disease, decreasing insulin resistance, decreasing plasma glucose, or decreasing weight, involving administering a PPARδ agonist or a pharmaceutical composition comprising a PPARδ agonist, are provided.

This application claims priority to U.S. Provisional Application No. 61/234,231, filed Aug. 14, 2009 and U.S. Provisional Application No. 61/251,655, filed Oct. 14, 2009, which are incorporated herein by reference in their entireties.

### FIELD

Provided herein are methods for treatment, prevention, or amelioration of one or more symptoms of a disease or condition related to disorders of insulin and/or glucose metabolism, inflammatory conditions, mitochondrial disease, muscle disorders, or pulmonary disorders, involving administering a PPARδ agonist or a pharmaceutical composition comprising a PPARδ agonist. In one embodiment, the disease or condition is selected from myopathy, inflammatory vascular diseases, Parkinson's and Alzheimer's diseases, systemic inflammatory disorders, renal ischemia, inflammatory rheumatic disorders, and inflammatory diseases of the lung. In another embodiment, methods for increasing oxidative muscle fibers, reducing mitochondria disease, decreasing insulin resistance, decreasing plasma glucose, or decreasing weight, involving administering a PPARδ agonist or a pharmaceutical composition comprising a PPARδ agonist, are provided.

### BACKGROUND

The peroxisome is a small organ present in cells of animals and plants, and its matrix contains various enzymes such as catalases. Various compounds such as fibrates, herbicides, and phthalic acid plasticizers are known as peroxisome proliferators, which induce proliferation of peroxisomes.

Isseman, et al. have identified a nuclear receptor which is activated by peroxisome proliferator and called it peroxisome proliferator activated receptor (PPAR) (Nature, 347:645-650, 1990). Since then three subtypes of PPAR, designated PPARα, PPARγ and PPARδ have been identified (Proc. Natl. Acad. Sci. USA, 91: 7335-7359, 1994).

Different compounds have been shown to modulate PPAR activity. The fibrates used as serum triglyceride (TG) lowering drugs modulate PPARα activity, and thiazolidine compounds (Troglitazone, Rosiglitazone, Pioglitazone) useful in the treatment of diabetes are known as ligands of PPARγ.

PPARδ agonists have been shown to increase the level of HDL particles and reduce the atherosclerotic plaque as the first acceptors of cholesterol from peripheral cells by reverse cholesterol transport. Pre-β HDL particles were first described by C. Fielding (Biochemistry 27(1):25-29 (1988)) and are small HDL discoidal particles with very few molecules of lipids, mainly phospholipids, and apoA-I. The mechanism of interaction between pre-β particles and cells is still largely unknown. Nevertheless, ABCA1 transporters seem to be involved in the cholesterol efflux from cells to pre-β HDL. Following the efflux, the pre-β HDL particles are further transformed into more mature and larger particles such as HDL₃ and HDL₂. The latter interact with liver cells for cholesterol elimination through the bile duct. It is noteworthy that this pathway, which is the reverse cholesterol transport, is the main, if not the only, cholesterol eliminaton pathway from the body. This pathway is also called reverse lipid transport since other lipids, such as oxidized lipids, are transported and cleared by the same mechanism.

Two different studies in humans using apoA-I Milano and human plasma apoA-I associated with small artificial HDL particles (JAMA 290(17):2292-2300 (2003); and JAMA 297(15):1675-1678 (2007)) have demonstrated the important role of pre-β HDL particles in plaque regression. Additionally, a recent post-hoc analysis of two large clinical trials by Steeg et al. (JACC 51:634-643 (2008)) found that when controlling for apoAI, HDL-C and apoB, elevated apoAI is a better predictor of decreased cardiovascular risk than HDL-C. The study further suggested that at very high HDL-C levels, HDL-C may actually increase risk.

By various other mechanisms, PPAR δ agonists are effective at preventing, reversing, or treating other types of inflammations and particularly diseases linked to lung inflammation. Using intravital microscopy in the mouse cremasteric microcirculation, Piqueras et al [CITE] have shown that activation of PPAR δ by its selective ligand GW501516 inhibited TNF-alpha induced leukocyte rolling flux, adhesion, and emigration in a dose-dependant manner. Moreover, PPARδ agonists reduced the expression of adhesion molecules such as ICAM-1, VCAM-1, and E-selectin in the cremasteric postcapillary venules. Similarly, rolling and adhesion of hPMNs under physiological flow on TNF-alpha-activated HUVECs were also inhibited markedly by GW501516. These inhibitory responses of GW501516 on activated endothelium were accompanied by a reduction in TNF-alpha induced endothelial GRO-release and VCAM-1, E-selectin, and ICAM-1 mRNA expression. Taken together, these results show that PPAR δ modulates acute inflammation in vivo and in vitro under flow by targeting the neutrophil-endothelial cell (J. Leukoc. Biol. 86, 2009).

Renal ischemia, also called nephric ischemia, is the deficiency of blood in one or both kidneys, or nephrons, usually due to functional constriction or actual obstruction of a blood vessel. Acute renal ischemia is associated with significant morbidity and mortality. There has been little progress in treating the disease over the last 50 years. Currently dialysis is the only effective therapy. A few reports have proposed a relationship between the activation of PPARα (Portilla et al., Am J. Physiol. Renal Physiol. 278: F667-F675 (2000)), PPARγ (Sivarajah et al., Am. J. Nephrol. 23: 267-276 (2003)) and δ (Letavernier et al. J. Am. Soc. Nephrol. 16: 2395-2402 (2005)) and protection from acute renal ischemia. It has been suggested that the protective effect of PPARδ may be due to its activation of the anti-apoptotic Akt signaling pathway and by promoting increased spreading of tubular epithelial cells.

PPAR α and δ expression is reactivated in the adult epidermis after various stimuli, resulting in keratinocyte proliferation and differentiation such as tetradecanoylphorbol acetate topical application, hair plucking, or skin wound healing. It was shown that PPAR δ mutant primary keratinocytes show impaired adhesion and migration properties, revealing PPAR α and δ activity in adult mouse epidermal repair (Michalik, L., J. Cell Biol, 2001. 154, (4), 799-814).

Some specific PPAR agonists have been shown effective in inducing peroxisomal and lipid metabolic gene expression in human keratinocytes. It has been shown that targeted deletion of PPARγ in follicular stem cells in mice causes a skin and hair phenotype that emulates scarring alopecia (Karnik et al. J Investigative Dermatology ((2009) 129, 1243-1257). These studies suggest that PPARγ is crucial for healthy pilosebaceous units and it is the loss of this function that triggers the pathogenesis of LPP. PPARδ agonist-targeted therapy may represent a new strategy in the treatment of these disorders.

Therapeutic use of certain peroxisome proliferator—activated receptor PPARα agonists (e.g., fibrates) for the treatment of dyslipidemia has infrequently been associated with the untoward side effect of myopathy (Faiola et al. Toxicol. Sci. 2008; 105: 384-394). Myopathy is a muscular disease in which the muscle fibers do not function properly resulting in muscular weakness. PPAR-δ agonists induced similar hepatic and skeletal muscle alterations as noted with some fibrates. PPAR-α KO and corresponding wild-type (WT) mice were administered toxicological dosages of a potent PPARδ agonist tool ligand GW0742, which also has weak PPAR-α agonist activity, or a potent PPAR-α agonist WY-14,643 for 10 days. Increases in liver weights and clinical chemistry indicators of skeletal muscle damage and/or liver injury were more pronounced in WT mice compared to KO mice administered the PPAR-δ agonist. Likewise, the incidence and severity of skeletal myopathy were greater in WT mice given GW0742 compared to KO mice. Ultrastructural and immunohistochemical analysis revealed significant peroxisome proliferation in muscle and liver of WT mice treated with each agonist, however, KO animals showed little or no evidence of hepatic and muscle peroxisome proliferation. PMP-70 protein expression in liver was consistent with these results. The hepatomegaly, hepatic and skeletal muscle peroxisome proliferation, and skeletal myopathy induced by this PPARδ ligand were predominantly mediated by its cross-activation of PPARα, though PPARδ agonism contributed slightly to these effects.

Mitochondrial diseases are disorders that affect the function of the mitochondria generally induced by mitochondrial DNA. Mitochondrial diseases take on unique characteristics because of the way the diseases are often inherited and because mitochondria are critical to cell function. The subclass of these diseases that have neuromuscular disease symptoms are often referred to as a mitochondrial myopathy. In addition to the mitochondrial myopathies, other examples include: diabetes mellitus, deafness, Leber's hereditary optic neuropathy (LHON), visual loss beginning in young adulthood, Wolff-Parkinson-White syndrome, multiple sclerosis-type disease, Leigh syndrome, subacute sclerosing encephalopathy, neuropathy, ataxia, retinitis pigmentosa, ptosis, and myoneurogenic gastrointestinal encephalopathy (MNGIE).

Many neurodegenerative diseases cause physical or functional alteration of mitochondria. This is the case for rare neurodegenerative disorders as well as extremely common age-related diseases, such as Alzheimer's and Parkinson's diseases. For some disorders, specific patterns of altered mitochondrial function or systemic mitochondrial dysfunction are demonstrable. Some disorders arise from mitochondrial DNA mutation, some arise from nuclear gene mutation, and for some the etiology is not definitively known. Swerdlow R H. (J Alzheimers Dis. 2009 Jun. 19, on-line edition) classifies neurodegenerative diseases using mitochondrial dysfunction as a unifying feature, and defines a group of disorders called neurodegenerative mitochondriopathies.

Beta-oxidation takes place in mitochondria. Fatty acids (FAs) are components of cell membrane, enzymes, and hormones and are one of the most important energy sources for organisms. There are several types of fatty acids oxidative degradation processes in the cell, namely alpha-, beta-, and omega-oxidation, which takes place in specialized cellular structures: mitochondria and peroxisomes. One known pathway is beta-oxidation taking place in the matrix of mitochondria. It is responsible for the degradation of straight-chain FAs. The pathway of beta-oxidation of fatty acids is comprised of at least 25 enzymes and specific transport proteins. Deficiencies in 18 of them have been demonstrated to cause diseases in humans. These diseases show a wide variety of symptoms, which can be expressed at random, one at a time, or in sets, characteristic of the individual rather than the metabolic character of the disease. Disorders of beta-oxidation are believed to cause about 1-3% of unexplained sudden infant deaths (SIDS). Acute fatty liver of pregnancy (AFLP) and the syndrome of hemolysis, elevated liver enzymes, and low platelets (HELLP syndrome), which have significant neonatal and maternal morbidity and mortality, have also been associated with beta-oxidation deficiency in fetuses. Moczulski D (Antioxid Redox Signal. 2009 May 7) and Yao Z et al. (Postepy Biochem. 2008; 54(2):161-8) summarized recent observations on disorders associated with fatty-acid oxidation, such as deficiencies of beta-oxidation enzymes, namely VLCAD, TFP and LCHAD, MCAD, MCKAT, M/SCHAD, and SCAD, and deficiencies of the enzymes TCP I, CT, and CPT II of the carnitine cycle.

Mitochondrial diseases in children are more frequently caused by mutations in nuclear DNA than in mitochondrial tDNA and are diagnosed by biochemical investigation of muscle biopsy and search for mitochondrial mutations. Special clinical phenotypes are associated with the mutations in SURF1 gene, in SCO2 gene and with mtDNA depletion syndromes. Leigh syndrome is the most common clinical presentation of various mitochondrial disorders during childhood (Pronicka E, Postepy Biochem. 2008; 54(2):161-8).

Thus, there is a need for methods of using PPARδ agonists for treating the above-referenced disorders and diseases.

### SUMMARY

In one embodiment, provided is a method of treatment, prevention, or amelioration of one or more symptoms of insulin resistance, involving administering a PPARδ agonist. Such methods reduce, alleviate or eliminate antihyperglycemic and insulin-sensitizing effects.

In another embodiment, provided is a method of treatment, prevention, or amelioration of one or more symptoms of disorders associated with increased oxidative muscle fibers, involving administering a PPAR-δ agonist.

In another embodiment, provided is a method of treatment, prevention, or amelioration of one or more symptoms of inflammation, involving administering a PPARδ agonist.

In another embodiment, provided is a method of treatment, prevention, or amelioration of one or more symptoms of diseases or disorders associated with functional constriction or actual obstruction of a kidney blood vessel, involving administering a PPARδ agonist. In these methods, the PPARδ agonist improves blood circulation in one or both kidneys.

In another embodiment, provided is a method of treatment, prevention, or amelioration of one or more symptoms of disorders associated with lung inflammation, involving administering a PPAR-δ agonist.

In another embodiment, provided is a method for treating diseases of the lung, including but not limited to, chronic obstructive airways disease (COAD), chronic obstructive pulmonary disease (COPD), adult onset asthma, emphysema or juvenile onset, and asthma, involving administering a PPARδ agonist.

In another embodiment, provided is a method for treating other inflammatory conditions where an inflammatory response is present such as inflammatory vascular diseases (including but not limited to atherosclerosis, coronary or peripheral vascular disease, myocardial infarction or stroke), inflammatory bowel disease (Crohn's disease and ulcerative colitis), systemic inflammatory disorders (Lupus Erythematosus) or inflammatory rheumatic disorders (including but not limited to rheumatoid arthritis or psoriatic joint disease), and inflammatory diseases of the lung, involving administering a PPARδ agonist.

In another embodiment, provided is a method for treating disorders or manifestations of insulin and glucose metabolism (including insulin resistance, diabetes, the metabolic syndrome, hypoglycemia, high blood pressure, obesity or dyslipidemia, protection of pancreatic beta cells and prevention of microvascular and macrovascular disorders), involving administering a PPARδ agonist.

In another embodiment, provided is a method for treating central or abdominal or visceral obesity, in which weight loss is required or desired, involving administering a PPARδ agonist.

In another embodiment, provided is a method for treating disorders of the kidney, including but not limited to, renal ischemia, involving administering a PPARδ agonist.

In another embodiment, provided is a method for treating mitochondrial disorders, including but not limited to, myoclonus twitching, epilepsy, ragged red fibers (RRF), hearing loss, exercise intolerance, dementia, and lactic acidosis, comprising administering a PPARδ agonist.

In another embodiment, provided is a method for treating hair loss comprising administering a PPARδ agonist.

In another embodiment, provided is a method for wound healing comprising administering a PPARδ agonist.

In another embodiment, in the methods provided, the use of a low dose of any selective PPARδ agonist with a selectivity of >500 over PPARα and PPARγ results that avoid the side effects associated with the use of PPARα and PPARγ agonists, or classical PPAR agonist side effects when used in conjuction to treatment of the disorders above. Exemplary compounds include but are not limited to GW-501516 (Ligand/GSK), RWJ-800025 (JNJ/Metabolex), KD-3010 (Kalypsys, Inc.), BAY 68-5042 (Bayer), and compounds described in Bratton, L. D. et al., Bioorg. Med. Chem. Lett. 2007 (web edition) and Kasuga, J. I. et al., Bioorg. Med. Chem. 2007 (web edition).

Examples of PPARδ compounds for use in the compositions and methods provided herein are described below.

In one embodiment, the compounds have the following general formula (I) or a salt thereof:

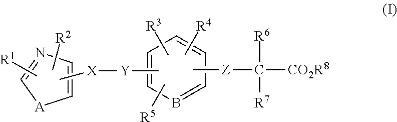

wherein:

R¹ is phenyl, naphthyl, pyridyl, thienyl, furyl, quinolyl or benzothienyl, any of which can have substituents selected from the group consisting of C₁₋₈ alkyl, C₁₋₈ alkyl having halogen, C₁₋₈ alkoxy, C alkoxy having halogen, C₂₋₈ alkenyl, C₂₋₄ alkynyl, halogen, C₂₋₇ acyl, benzoyl, hydroxyl, nitro, amino, phenyl and pyridyl;

R² is C₂₋₈ alkyl, C₁₋₈ alkyl having halogen, C₂₋₈ alkenyl, C₂₋₈ alkynyl, 3-7 membered cycloalkyl, C₁₋₈ alkyl having 3-7 membered cycloalkyl, or C₁₋₆ alkyl substituted with phenyl, naphthyl or pyridyl, any of which can have substituents selected from the group consisting of C₁₋₈ alkyl, C₁₋₈ alkyl having halogen, C₁₋₈ alkoxy, C₁₋₈ alkoxy having halogen, C₂₋₈ alkenyl, C₂₋₈ alkynyl, halogen, C₂₋₇ acyl, benzoyl, hydroxyl, nitro, amino, phenyl and pyridyl;

A is oxygen, sulfur or NR⁹ in which R⁹ is hydrogen or C₁₋₈ alkyl;

X is a C₁₋₈ alkylene chain which can have substituents selected from the group consisting of C₁₋₈ alkyl, C₁₋₈ alkoxy and hydroxyl and which can contain a double bond;

Y is C(═=O), C(═±N—-OR¹⁰), CH(OR¹¹), CH═=CH, C—-C, or C(═=CH₂) in which each of R¹⁰ and R¹¹ is hydrogen or C₁₋₈ alkyl;

each of R³, R⁴ and R⁵ is hydrogen, C₁₋₈ alkyl, C₁₋₈ alkyl having halogen, C₁₋₈ alkoxy, C₁₋₈ alkoxy having halogen, C₂₋₈ alkenyl, C₂₋₈ alkynyl, halogen, C₂₋₇ acyl, benzoyl, hydroxyl, nitro, amino, phenyl, or pyridyl;

B is CH or nitrogen;

Z is oxygen or sulfur;

each of R⁶ and R⁷ is hydrogen, C₁₋₈ alkyl, C₁₋₈ alkyl having halogen; and

R⁸ is hydrogen or C₁₋₈ alkyl;

provided that at least one of R³, R⁴ and R⁵ is not hydrogen.

Also provided is an activator of peroxisome proliferator activated receptor δ, which contains as an effective component a compound of the formula (I) or a salt thereof.

In another embodiment, a compound has the following general formula (II) or a salt thereof

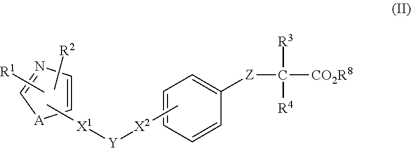

wherein:

each of R¹ and R² independently is a hydrogen atom, a halogen atom, nitro, an alkyl group having 1-8 carbon atoms, an alkoxy group having 1-8 carbon atoms, an alkyl group having 1-8 carbon atoms which has 1 to 3 halogen substituents, an alkoxy group having 1-8 carbon atoms which has 1 to 3 halogen substituents, an alkenyl group having 2-8 carbon atoms, an alkynyl group having 2-8 carbon atoms, a 3-7 membered cycloalkyl group, an alkyl group having 1-8 carbon atom which has a 3-7 membered cycloalkyl substituent, an aryl group having 6-10 carbon atoms which optionally has a substituent, an arylalkyl group which has a C₆₋₁₀ aryl portion and C₁₋₈ alkyl portion, a heterocyclic group which optionally has a substituent or a heterocyclic-alkyl group having an alkyl group of 1-8 carbon atoms;

A is an oxygen atom, a sulfur atom, or NR³ in which R³ is a hydrogen atom or an alkyl group having 1-8 carbon atoms;

each of X and Z independently is —C(═O)—, —C(═O)NH—, —C(═N—OR⁴)—, —CH(OR⁵)—, —NH(C═O)—, —NHSO₂—, —SO₂NH—, —CH═CH—, or a bond in which each of R⁴ and R⁵ is a hydrogen atom or an alkyl group having 1-8 carbon atoms; and

Y is an alkylene chain having 1-8 carbon atoms.

Also provided is an activator of peroxisome proliferator activated receptor δ, which contains as an effective component a compound of the formula (II) or a salt thereof.

In yet another embodiment, the compound has the following formula (III) or a salt thereof:

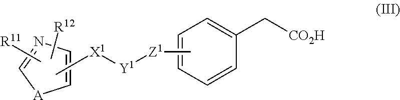

wherein:

each of R¹¹ and R¹² independently is a hydrogen atom, a halogen atom, nitro, hydroxyl, amino, an alkyl group having 1-8 carbon atoms, an alkoxy group having 1-8 carbon atoms, an alkyl group having 1-8 carbon atoms which has 1 to 3 halogen substituents, an alkoxy group having 1-8 carbon atoms which has 1 to 3 halogen substituents, an alkenyl group having 2-8 carbon atoms, an alkynyl group having 2-8 carbon atoms, a 3-7 membered cycloalkyl group, an alkyl group having 1-8 carbon atoms which has a 3-7 membered cycloalkyl substituent, or a phenyl, naphthyl, benzyl, phenethyl, pyridyl, thienyl, furyl, quinolyl, or benzothienyl group which optionally has a substituent selected from the group consisting of a halogen atom, nitro, hydroxyl, amino, an alkyl group having 1-8 carbon atoms, an alkoxy group having 1-8 carbon atoms, an alkyl group having 1-8 carbon atoms which has 1 to 3 halogen substituents, an alkoxy group having 1-8 carbon atoms which has 1 to 3 halogen substituents, an alkenyl group having 2-8 carbon atoms, an alkynyl group having 2-8 carbon atoms, a 3-7 membered cycloalkyl group, an alkyl group having 1-8 carbon atoms which has a 3-7 membered cycloalkyl substituent, phenyl and pyridyl;

each of X¹ and Z¹ independently is —C(═O)—, —C(═O)NH—, —C(═N—OR¹⁴)—, —CH(OR¹⁵)—, —NH(C═O)—, —NHSO₂—SO₂NH—, —CH═CH—, or a bond in which each of R¹⁴ and R¹⁵ is a hydrogen atom or an alkyl group having 1-8 carbon atoms; and

Y¹ is an alkylene chain having 1-8 carbon atoms.

In another embodiment, provided is an activator of peroxisome proliferator activated receptor which contains as an effective component a phenylacetic acid derivative of the formula (III) or their salts.

In another embodiment, the compound has the following general formula (IV) or a salt thereof:

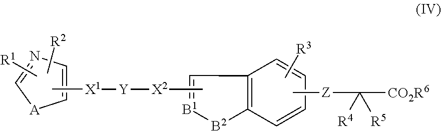

wherein:

A is O, S or NR⁷ in which R⁷ is hydrogen or C₁₋₈ alkyl;

B¹ is CW or N in which W is hydrogen or a bond; B² is O, S or NR⁸ in which R⁸ is hydrogen or C₁₋₈ alkyl;

each of X¹ and X² is O, S, NH, NHC(═O), C(═=O), C(═N—OR⁹, CH(OR¹⁰), C═C, c≡C or a bond in which each of R⁹ and R¹⁰ is hydrogen or C₁₋₈ alkyl;

Y is a C₁₋₈ alkylene chain, which can be substituted with C₁₋₈ alkyl or C₁₋₈ alkyl substituted with 1-3 halogens;

Z is NH, O or S;

R¹ is aryl, which can be substituted with a group or atom selected from the group consisting of C₁₋₈ alkyl, C₁₋₈ alkoxy, C₁₋₈ alkyl substituted with 1-3 halogens, hydroxyl, nitro, amino, phenyl, pyridyl and halogen, or a heterocyclic group having five to eight membered ring comprising one to three hetero atoms selected from the group consisting of nitrogen, oxygen and sulfur and the other atoms consisting of carbon (benzene ring can be condensed with the heterocyclic ring);

R² is C₂₋₈ alkyl, C₁₋₈ alkyl substituted with 1-3 halogens, C₃₋₇ cycloalkyl, C₂₋₈ alkenyl, C₂₋₈ alkynyl, alkyl (comprising C₁₋₈ alkyl moiety) substituted with aryl, which can be substituted with a group or atom selected from the group consisting of C₁₋₈ alkyl, C₁₋₈ alkoxy, C₁₋₈ alkyl substituted with 1-3 halogens, hydroxyl, nitro, amino, phenyl, pyridyl and halogen, or C₁₋₄ alkyl substituted with a heterocyclic group having five to eight membered ring having one to three heteroatoms selected from the group consisting of nitrogen, oxygen and sulfur and the other atoms consisting of carbon;

R³ is halogen, trifluoromethyl, C₁₋₈ alkyl, C₂₋₈ alkenyl or C₂₋₈ alkynyl;

each of R⁴ and R⁵ is hydrogen, C₁₋₈ alkyl or C₁₋₈ alkyl substituted with 1-3 halogens; and R⁶ is hydrogen, C₁₋₈ alkyl substituted with amino, C₁₋₈ alkyl or alkali metal;

provided that each of Z and R³ is attached to the benzene ring, and X² is not attached to the benzene ring.

Also provided is an activator of peroxisome proliferator activated receptor δ, which contains as an effective component a compound of the formula (IV) or a salt thereof.

In another embodiment, the compound has the following general formula (V) or a salt thereof

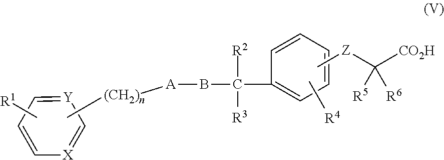

wherein:

R¹ and R⁴ are the same or different and each represents a hydrogen atom, an alkyl group having 1 to 8 carbon atoms, an alkenyl group having 2 to 8 carbon atoms, an alkynyl group having 2 to 8 carbon atoms, an alkoxy group having 1 to 8 carbon atoms, a halogen atom, an alkyl group having 1 to 8 carbon atoms and a halogen atom substituent, an alkoxy group having 1 to 8 carbon atoms and a halogen atom substituent, a hydroxyl group, a nitro group, an acyl group having 2 to 8 carbon atoms, an aryl group having 6 to 10 carbon atoms, or a 5- or 6-membered heterocyclic group;

R² represents a hydrogen atom;

R³ represents an alkyl group having 1 to 8 carbon atoms, or R³ is combined with R² to represent ═O or ═C(R⁷)(R⁸) in which R⁷ and R⁸ are the same or different and each represents a hydrogen atom or an alkyl group having 1 to 8 carbon atoms;

R⁵ and R⁶ are the same or different and each represents a hydrogen atom, an alkyl group having 1 to 8 carbon atoms, or an alkyl group having 1 to 8 carbon atoms and a halogen atom substituent;

X and Y are the same or different and each represents CH or N;

Z represents an oxygen atom or a sulfur atom;

A represents a 5-membered heterocyclic group selected from the group consisting of pyrazole, thiophene, furan and pyrrole which optionally has an alkyl substituent having 1 to 8 carbon atoms which has a substituent selected from the group consisting of an alkyl group having 1 to 8 carbon atoms, a 3- to 7-membered cycloalkyl group, an alkenyl group having 2 to 8 carbon atoms, an alkynyl group having 2 to 8 carbon atoms, an alkoxy group having 1 to 8 carbon atoms, an alkyl group which has 1 to 8 carbon atoms and a 3- to 7-membered cycloalkyl group substituent, an alkyl group having 1 to 8 carbon atoms and a halogen atom substituent, an alkoxy group having 1 to 8 carbon atoms and a halogen atom substituent, an aryl group having 6 to 10 carbon atoms, a 5- or 6-membered heterocyclic group, an aralkyl group having an aryl moiety of 6 to 10 carbon atoms and an alkylene moiety of 1 to 8 carbon atoms, and 5- or 6-membered heterocyclic group;

B represents an alkylene chain having 1 to 8 carbon atoms which optionally has a substituent selected from the group consisting of an alkyl group having 1 to 8 carbon atoms, a 3- to 7-membered cycloalkyl group, an alkenyl group having 2 to 8 carbon atoms, an alkynyl group having 2 to 8 carbon atoms, an alkoxy group having 1 to 8 carbon atoms, a halogen atom, an alkyl group having 1 to 8 carbon atoms and a halogen atom substituent, and an alkoxy group having 1 to 8 carbon atoms and a halogen atom substituent, the alkylene group optionally having a double bond in the case that the alkylene group has 2 to 6 carbon atoms; and

n is an integer of 0 to 5.

In another embodiment, provided are compounds having the following formula (VI) or salts thereof:

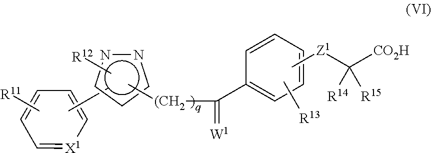

wherein:

R¹¹ and R¹³ are the same or different and each represents a hydrogen atom, an alkyl group having 1 to 8 carbon atoms, an alkenyl group having 2 to 8 carbon atoms, an alkynyl group having 2 to 8 carbon atoms, an alkoxy group having 1 to 8 carbon atoms, a halogen atom, an alkyl group having 1 to 8 carbon atoms and a halogen atom substituent, an alkoxy group having 1 to 8 carbon atoms and a halogen atom substituent, a hydroxyl group, a nitro group, an acyl group having 2 to 8 carbon atoms, an aryl group having 6 to 10 carbon atoms, or a 5- or 6-membered heterocyclic group;

R¹² represents a hydrogen atom, an alkyl group having 1 to 8 carbon atoms, a 3- to 7-membered cycloalkyl group, an alkenyl group having 2 to 8 carbon atoms, an alkynyl group having 2 to 8 carbon atoms, an alkoxy group having 1 to 8 carbon atoms, an alkyl group having 1 to 8 carbon atoms and a 3- to 7-membered cycloalkyl group substituent, an alkyl group having 1 to 8 carbon atoms and a halogen atom substituent, an alkoxy group having 1 to 8 carbon atoms and a halogen atom substituent, an aryl group having 6 to 10 carbon atoms, a 5- or 6-membered heterocyclic group, an aralkyl group having an aryl moiety of 6 to 10 carbon atoms and an alkylene moiety of 1 to 8 carbon atoms, or an alkyl group having 1 to 8 carbon atoms and a 5- or 6-membered heterocyclic substituent;

R¹⁴ and R¹⁵ are the same or different and each represents a hydrogen atom, an alkyl group having 1 to 8 carbon atoms, or an alkyl group having 1 to 8 carbon atoms and a halogen atom substituent;

X¹ represents CH or N;

Z¹ represents an oxygen atom or a sulfur atom;

W¹ represents an oxygen atom or CH₂; and

q is an integer of 2 to 4.

In another embodiment, provided are compounds having the following formula (VII) or salts thereof:

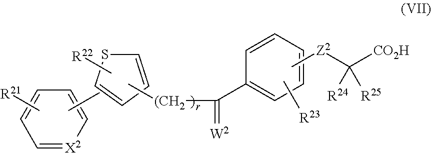

wherein:

R²¹ and R²³ are the same or different and each represents a hydrogen atom, an alkyl group having 1 to 8 carbon atoms, an alkenyl group having 2 to 8 carbon atoms, an alkynyl group having 2 to 8 carbon atoms, an alkoxy group having 1 to 8 carbon atoms, a halogen atom, an alkyl group having 1 to 8 carbon atoms and a halogen atom substituent, an alkoxy group having 1 to 8 carbon atoms and a halogen atom substituent, a hydroxyl group, a nitro group, an acyl group having 2 to 8 carbon atoms, an aryl group having 6 to 10 carbon atoms, or a 5- or 6-membered heterocyclic group;

R²² represents a hydrogen atom, an alkyl group having 1 to 8 carbon atoms, a 3- to 7-membered cycloalkyl group, an alkenyl group having 2 to 8 carbon atoms, an alkynyl group having 2 to 8 carbon atoms, an alkoxy group having 1 to 8 carbon atoms, an alkyl group having 1 to 8 carbon atoms and a 3- to 7-membered cycloalkyl group substituent, an alkyl group having 1 to 8 carbon atoms and a halogen atom substituent, an alkoxy group having 1 to 8 carbon atoms and a halogen atom substituent, an aryl group having 6 to 10 carbon atoms, a 5- or 6-membered heterocyclic group, an aralkyl group having an aryl moiety of 6 to 10 carbon atoms and an alkylene moiety of 1 to 8 carbon atoms, or an alkyl group having 1 to 8 carbon atoms and a 5- or 6-membered heterocyclic substituent;

R²⁴ and R²⁵ are the same or different and each represents a hydrogen atom, an alkyl group having 1 to 8 carbon atoms, or an alkyl group having 1 to 8 carbon atoms and a halogen atom substituent;

X² represents CH or N;

Z² represents an oxygen atom or a sulfur atom;

W² represents an oxygen atom or C₁₋₁₂; and

r is an integer of 2 to 4.

In still another embodiment, provided is an activator for peroxisome proliferator activated receptor δ containing a compound of the formula (V), (VI) or (VII) as an effective component.

In another embodiment, provided are compounds having the following formula (VIII) or salts thereof:

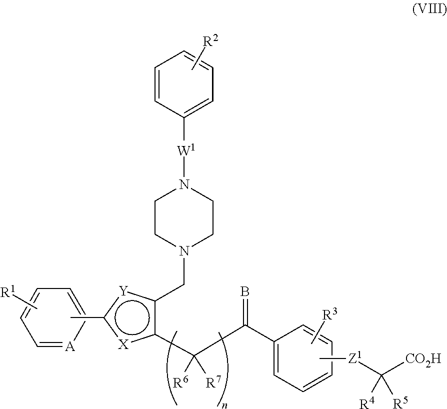

wherein:

A represents CH or a nitrogen atom;

B represents an oxygen atom or C(R⁸)(R⁹) in which each of R⁸ and R⁹ independently represents a hydrogen atom or an alkyl group having 1 to 8 carbon atoms;

W¹ represents a bond, C(═O), or (—C(R¹⁰)(R¹¹)—)ₘ in which each of R¹⁰ and R¹¹ independently a hydrogen or an alkyl group having 1 to 8 carbon atoms and m is an integer of 1 to 3;

X and Y differ from each other, and each represents an oxygen atom, a sulfur atom, a nitrogen atom, or CR¹² in which R¹² represents a hydrogen atom or an alkyl group having 1 to 8 carbon atoms;

Z¹ represents a bond, an oxygen atom, a sulfur atom, or C(R¹³)(R¹⁴) in which each of R¹³ and R¹⁴ independently represents a hydrogen atom or an alkyl group having 1 to 8 carbon atoms;

each of R¹, R² and R³ independently represents a hydrogen atom, an alkyl group having 1 to 8 carbon atoms, an alkenyl group having 2 to 8 carbon atoms, an alkynyl group having 2 to 8 carbon atoms, an alkoxy group having 1 to 8 carbon atoms, a halogen atom, an alkyl group having 1 to 8 carbon atom which is substituted with a halogen atom, an alkoxy group having 1 to 8 carbon atom which is substituted with a halogen atom, hydroxyl, nitro, an acyl group having 2 to 8 carbon atoms, an aryl group having 6 to 10 carbon atoms, or a 5- or 6-membered heterocyclic group;

each of R⁴ and R⁵ independently represents a hydrogen atom, an alkyl group having 1 to 8 carbon atoms, or an alkyl group having 1 to 8 carbon atoms which is substituted with a halogen atom;

each of R⁶ and R⁷ independently represents a hydrogen atom, an alkyl group having 1 to 8 carbon atoms, an alkenyl group having 2 to 8 carbon atoms, an alkynyl group having 2 to 8 carbon atoms, or an alkyl group having 1 to 8 carbon atoms which is substituted with a halogen atom; and

n represents an integer of 1 to 5.

In another embodiment, provided are compounds having the following formula (IX) or salts thereof:

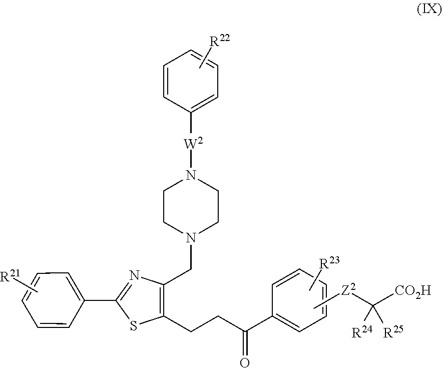

wherein:

W² represents a bond, C(═O), or —CH₂;

Z² represents an oxygen atom or a sulfur atom;

each of R²¹, R²² and R²³ independently represents a hydrogen atom, an alkyl group having 1 to 8 carbon atoms, an alkenyl group having 2 to 8 carbon atoms, an alkynyl group having 2 to 8 carbon atoms, an alkoxy group having 1 to 8 carbon atoms, a halogen atom, an alkyl group having 1 to 8 carbon atom which is substituted with a halogen atom, an alkoxy group having 1 to 8 carbon atom which is substituted with a halogen atom, hydroxyl, nitro, an acyl group having 2 to 8 carbon atoms, an aryl group having 6 to 10 carbon atoms, or a 5- or 6-membered heterocyclic group;

each of R²⁴ and R²⁵ independently represents a hydrogen atom, an alkyl group having 1 to 8 carbon atoms, or an alkyl group having 1 to 8 carbon atoms which is substituted with a halogen atom.

In still another embodiment, provided is an activator for peroxisome proliferator-activated receptor containing a compound of the formulas (VIII) or (IX) as an effective component.

In another embodiment, provided are compounds having the following formula (X) or salts thereof:

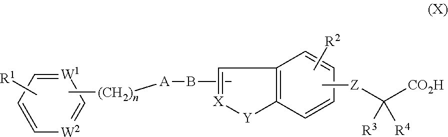

wherein:

each of W¹ and W² independently represents a nitrogen atom or CH′

X represents a nitrogen atom or CH;

Y represents an oxygen atom or a sulfur atom;

Z represents a bond, an oxygen atom, a sulfur atom or NR^{S}, in which R⁵ represents a hydrogen atom or an alkyl group having 1 to 8 carbon atoms;

each of R¹ and R² independently represents a hydrogen atom, a halogen atom, a hydroxyl group, a nitro group, an amino group, an alkyl group having 1 to 8 carbon atoms, a 3- to 7-membered cycloalkyl group, an alkenyl group having 2 to 8 carbon atoms, an alkynyl group having 2 to 8 carbon atoms, an alkoxy group having 1 to 8 carbon atoms, an alkyl group having 1 to 8 carbon atoms and a 3- to 7-membered cycloalkyl substituent, an alkyl group having 1 to 8 carbon atoms and a halogen substituent, an alkoxy group having 1 to 8 carbon atoms and a halogen substituent, an aryl group having 6 to 10 carbon atoms, 5- or 6-membered heterocyclic group, an aralkyl group having an aryl moiety of 6 to 10 carbon atoms and an alkylene moiety of 1 to 8 carbon atoms, or an alkyl group having 1 to 8 carbon atoms and a 5- or 6-membered heterocyclic substituent;

each of R³ and R⁴ independently represents a hydrogen atom, an alkyl group having 1 to 8 carbon atoms, or an alkyl group having 1 to 8 carbon atoms and a halogen substituent;

A represents a 5-membered hetero ring selected from the group consisting of pyrazole, thiophene, furan, isoxazole, isothiazole and pyrrole, in which the 5-membered hetero ring may have a substituent selected from the group consisting of a halogen atom, a hydroxyl group, a nitro group, an amino group, an alkyl group having 1 to 8 carbon atoms, a 3- to 7-membered cycloalkyl group, an alkenyl group having 2 to 8 carbon atoms, an alkynyl group having 2 to 8 carbon atoms, an alkoxy group having 1 to 8 carbon atoms, an alkyl group having 1 to 8 carbon atoms and a 3- to 7-membered cycloalkyl substituent, an alkyl group having 1 to 8 carbon atoms and a halogen substituent, an alkoxy group having 1 to 8 carbon atoms and a halogen substituent, an aryl group having 6 to 10 carbon atoms, a 5- or 6-membered heterocyclic group, an aralkyl group having an aryl moiety of 6 to 10 carbon atoms and an alkylene moiety of 1 to 8 carbon atoms, and an alkyl group having 1 to 8 carbon atoms and a 5- or 6-membered heterocyclic substituent;

B represents a bond or an alkylene chain having 1 to 8 carbon atoms which may have a substituent selected from the group consisting of an alkyl group having 1 to 8 carbon atoms, a 3- to 7-membered cycloalkyl group, an alkoxy group having 1 to 8 carbon atoms and a halogen substituent and further may have a double or triple bond; and

n is an integer of 0 to 3.

In another embodiment, provided are compounds having the following formula (XI) or salts thereof:

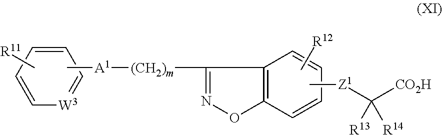

wherein:

W³ represents a nitrogen atom or CH;

Z¹ represents an oxygen atom or a sulfur atom;

each of R¹¹ and R¹² independently represents a hydrogen atom, a halogen atom, a hydroxyl group, a nitro group, an amino group, an alkyl group having 1 to 8 carbon atoms, an alkoxy group having 1 to 8 carbon atoms, an alkyl group having 1 to 8 carbon atoms and a halogen substituent, or an alkoxy group having 1 to 8 carbon atoms and a halogen substituent;

each of R¹³ and R¹⁴ independently represents a hydrogen atom or an alkyl group having 1 to 8 carbon atoms;

A¹ represents pyrazole or thiophene which may have a substituent selected from the group consisting of a halogen atom, a hydroxyl group, a nitro group, an amino group, an alkyl group having 1 to 8 carbon atoms, an alkoxy group having 1 to 8 carbon atoms, an alkyl group having 1 to 8 carbon atoms and a halogen substituent, or an alkoxy group having 1 to 8 carbon atoms and a halogen substituent; and

m is an integer of 2 to 4.

In a further embodiment, provided is an activator for peroxisome proliferator activated receptor δ containing a compound of the formulas (X) or (XI) as an effective component.

In another embodiment, provided are compounds having the following formula (XII) or salts thereof:

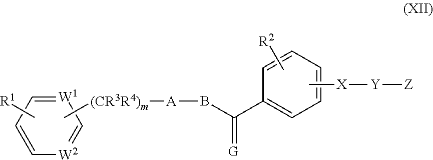

wherein:

each of W¹ and W² independently is CH or nitrogen;

X is NR⁵ or CR⁶R⁷, wherein R⁵ is hydrogen, C₁₋₈ alkyl, C₁₋₈ alkyl substituted with halogen, C₁₋₈ alkyl substituted with C₁₋₈ alkoxy, cycloalkyl of three-membered to seven-membered ring, C₁₋₈ alkyl substituted with cycloalkyl of three-membered to seven-membered ring, C₁₋₈ alkyl substituted with phenyl, C₂₋₈ acyl, or C₂₋₈ alkenyl, and each of R⁶ and R⁷ independently is hydrogen or C₁₋₈ alkyl;

Y is —(CR⁸R⁹)ₙ—, wherein each of R⁸ and R⁹ independently is hydrogen or C₁₋₈ alkyl, and n is 1 to 4; or

X and Y are combined to form —CR¹⁰═CR¹¹— or ethynylene, wherein each of R¹⁹ and R¹¹ independently is hydrogen or C₁₋₈ alkyl;

Z is carboxyl or tetrazolyl;

G is O, S or CR¹²R¹³, wherein each of R¹² and R¹³ independently is hydrogen or C₁₋₈ alkyl;

A is a five-membered heterocyclic ring selected from the group consisting of thiazole, oxazole, imidazole, pyrazole, thiophene, furan, and pyrrole, which can be substituted with a substituent selected from the group consisting of C₁₋₈ alkyl, C₂₋₈ alkenyl, C₂₋₈ alkynyl, C₁₋₈ alkoxy, halogen, C₁₋₈ alkyl substituted with halogen, C₁₋₈ alkoxy substituted with halogen, hydroxyl, nitro, C₂₋₈ acyl, C₆₋₁₀ aryl, and a five-membered or six-membered heterocyclic group;

B is a C₁₋₈ alkylene, C₂₋₈ alkenylene or C₂₋₈ alkynylene chain, wherein the chain can be substituted with a substituent selected from the group consisting of C₁₋₈ alkyl, cycloalkyl of three-membered to seven-membered ring, C₁₋₈ alkoxy, and halogen;

each of R¹ and R² independently is hydrogen, C₁₋₈ alkyl, C₂₋₈ alkenyl, C₂₋₈ alkynyl, C₁₋₈ alkoxy, halogen, C₁₋₈ alkyl substituted with halogen, C₁₋₈ alkoxy substituted with halogen, hydroxyl, nitro, C₂₋₈ acyl, C₆₋₁₀ aryl, or a five-membered or six-membered heterocyclic group;

each of R³ and R⁴ independently is hydrogen or C₁₋₈ alkyl; and

m is an integer of 0 to 3.

In another embodiment, provided are compounds having the following formula (XIII) or salts thereof:

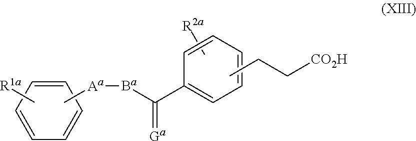

wherein:

wherein G^{a} is O, S or CH₂;

A^{a} is five-membered heterocyclic ring selected from the group consisting of thiazole, oxazole, and thiophene, which can be substituted with a substituent selected from the group consisting of C₁₋₈ alkyl, C₁₋₈ alkoxy, halogen, C₁₋₈ alkyl substituted with halogen, C₁₋₈ alkoxy substituted with halogen, hydroxyl, nitro, and C₂₋₈ acyl;

B^{a} is a C₁₋₈ alkylene or C₂₋₈ alkenylene chain; and

each of R^{1a} and R^{2a} independently is hydrogen, C₁₋₈ alkyl, C₁₋₈ alkoxy, halogen, C₁₋₈ alkyl substituted with halogen, C₁₋₈ alkoxy substituted with halogen, hydroxyl, nitro, or C₂₋₈ acyl.

In another embodiment, provided are compounds having the following formula (XIV) or salts thereof:

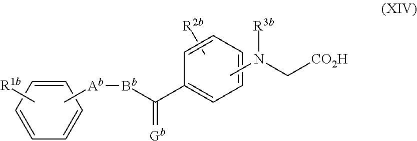

wherein:

G is O, S Or CH₂;

A is a five-membered heterocyclic ring selected from the group consisting of thiazole, oxazole, and thiophene, which can be substituted with a substituent selected from the group consisting of C₁₋₈ alkyl, C₁₋₈ alkoxy, halogen, C₁₋₈ alkyl substituted with halogen, C₁₋₈ alkoxy substituted with halogen, hydroxyl, nitro, and C₂₋₈ acyl;

B^{b} is a C₁₋₈ alkylene or C₂₋₈ alkenylene chain;

each of R^{1b} and R^{2b} independently is hydrogen, C₁₋₈ alkyl, C₁₋₈ alkoxy, halogen, C₁₋₈ alkyl substituted with halogen, C₁₋₈ alkoxy substituted with halogen, hydroxyl, nitro, or C₂₋₈ acyl; and

R^{3b} is hydrogen or C₁₋₈ alkyl.

Also provided is an activator of peroxisome proliferator activated receptor δ which contains as an effective component a compound having the formula (XII), (XIII), or (XIV) or a salt thereof.

In another embodiment, a compound has the following general formula (XV) or a salt thereof:

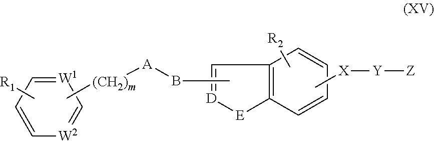

wherein:

each of W¹ and W² is independently CH or N;

X is NR³ or CR⁴R⁵, in which R³ is an alkyl group having 1 to 8 carbon atoms, an alkyl group having 1 to 8 carbon atoms and a halogen atom substituent, an alkyl group having 1 to 8 carbon atoms substituted with an alkoxy group having 1 to 8 carbon atoms, an alkyl group having 1 to 8 carbon atoms substituted with a 3-7 membered cycloalkyl group, an alkyl group having 1 to 8 carbon atoms substituted with a phenyl group, an acyl group having 2 to 8 carbon atoms or an alkenyl group having 2 to 8 carbon atoms;

each of R⁴ and R⁵ is independently H or an alkyl group having 1 to 8 carbon atoms;

Y is —(CR⁶R⁷)ₙ—, in which each of R⁶ and R⁷ is independently H or an alkyl group having 1 to 8 carbon atoms, and n is an integer of 1 to 4;

Z is a carboxylic group or a tetrazolyl group;

A is a 5 or 6-membered-heterocyclic group selected from the group consisting of thiazole, oxazole, imidazole, pyrazole, thiophene, furan, pyrrole, pyridine or pyrimidine, or a phenyl group, which may have a substituent selected from the group consisting of an alkyl group having 1 to 8 carbon atoms, a 3-7 membered cycloalkyl group, an alkenyl group having 2 to 8 carbon atoms, an alkynyl group having 2 to 8 carbon atoms, an alkoxyl group having 1 to 8 carbon atoms, an alkyl group having 1 to 8 carbon atoms substituted with a 3-7 membered cycloalkyl group, an alkyl group having 1 to 8 carbon atoms and a halogen atom substituent, an alkoxy group having 1 to 8 carbon atoms and a halogen atom substituent, an aryl group having 6 to 10 carbon atoms, a 5 or 6-membered heterocyclic group, aralkyl group comprising an aryl group having 6 to 10 carbon atoms and an alkyl group having 1 to 8 carbon atoms or an alkyl group having 1 to 8 carbon atoms substituted with a 5 or 6-membered heterocyclic group;

B is a bond or an alkylene chain having 1 to 8 carbon atoms which may have a substituent selected from the group consisting of an alkyl group having 1 to 8 carbon atoms, a 3-7 membered cycloalkyl group, an alkoxy group having 1 to 8 carbon atoms or a halogen atom and which may have a double bond or triple bond when the carbon number of alkylene chain is 2 or more;

D is N or CH;

E is O or S;

each of R¹ and R² is independently H, an alkyl group having 1 to 8 carbon atoms, an alkenyl group having 2 to 8 carbon atoms, an alkynyl group having 2 to 8 carbon atoms, an alkoxy group having 1 to 8 carbon atoms, a halogen atom, an alkyl group having 1 to 8 carbon atoms and a halogen atom substituent, an alkoxy group having 1 to 8 carbon atoms and a halogen atom substituent, hydroxyl, nitro, an acyl group having 2 to 8 carbon atoms, an aryl group having 6 to 10 carbon atoms or a 5 or 6-membered heterocyclic group; and

m is an integer of 0 to 3.

In one embodiment, the compounds having the formula (XV), wherein:

both W¹ and W² are CH;

X is CR⁴R⁵, CH₂, or NR³, wherein R³ is an alkyl group having 1 to 8 carbon atoms. In another embodiment, R³ is a methyl group;

Y is CH₂;

Z is a carboxylic group;

A is thiazole or oxazole which may have a substituent selected from the group consisting of an alkyl group having 1 to 8 carbon atoms, an alkenyl group having 2 to 8 carbon atoms, an alkynyl group having 2 to 8 carbon atoms, an alkyl group having 1 to 8 carbon atoms and a halogen atom substituent, an aryl group having 6 to 10 carbon atoms or a 5 or 6-membered heterocyclic group; pyrazole which may have a substituent selected from the group consisting of an alkyl group having 1 to 8 carbon atoms, an alkenyl group having 2 to 8 carbon atoms, an alkynyl group having 2 to 8 carbon atoms, an alkyl group having 1 to 8 carbon atoms and a halogen atom substituent, an aryl group having 6 to 10 carbon atoms or a 5 or 6-membered heterocyclic group;

B is an ethylene chain

D is N;

E is O;

each of R¹ and R² is independently H, an alkyl group having 1 to 8 carbon atoms, an alkenyl group having 2 to 8 carbon atoms, an alkoxy group having 1 to 8 carbon atoms, a halogen atom, an alkyl group having 1 to 8 carbon atoms and a halogen atom substituent or an alkoxy group having 1 to 8 carbon atoms and a halogen atom substituent; and

m is O.

In another embodiment, provided is a compound having the general formula (XVI) or a pharmaceutically acceptable salt thereof,

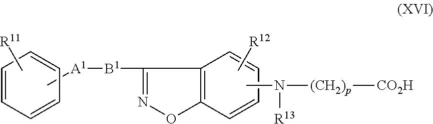

wherein:

R¹³ is an alkyl group having 1 to 8 carbon atoms or an alkyl group having 1 to 8 carbon atoms and a halogen atom substituent;

p is an integer of 1 to 4;

A¹ is thiazole, oxazole, pyridine, pyrimidine or phenyl which may have a substituent selected from the group consisting of an alkyl group having 1 to 8 carbon atoms or an alkyl group having 1 to 8 carbon atoms and a halogen atom substituent;

B¹ is an alkylene chain having 2 to 4 carbon atoms; and

each of R¹¹ and R¹² is independently H, an alkyl group having 1 to 8 carbon atoms, a halogen atom, an alkyl group having 1 to 8 carbon atoms and a halogen atom substituent. wherein N(R¹³)((CH₂)ₚ—CO₂H) is attached to the 6th position of benzisoxazole.

In another embodiment, the compounds having the formula (XVI), wherein:

R¹³ is a methyl group;

p is 1;

A¹ is thiazole, oxazole or phenyl which may have a substituent selected from the group consisting of an alkyl group having 1 to 8 carbon atoms or an alkyl group having 1 to 8 carbon atoms and a halogen atom substituent;

B¹ is an ethylene chain;

R¹¹ is an alkyl group having 1 to 8 carbon atoms, a halogen atom or an alkyl group having 1 to 8 carbon atoms and a halogen atom substituent; and

R¹² is H, an alkyl group having 1 to 8 carbon atoms or an alkyl group having 1 to 8 carbon atoms and a halogen atom substituent.

In yet another embodiment, provided is a compound having the general formula (XVI),

wherein:

R¹³ is an alkyl group having 1 to 8 carbon atoms;

p is 1;

A¹ is thiazole which may have an alkyl group having 1 to 8 carbon atoms as a substituent;

B¹ is an ethylene chain;

R¹¹ is an alkyl group having 1 to 8 carbon atoms, a halogen atom or an alkyl group having 1 to 8 carbon atoms and a halogen atom substituent; and

R¹² is H, an alkyl group having 1 to 8 carbon atoms or an alkyl group having 1 to 8 carbon atoms and a halogen atom substituent.

In yet another embodiment, provided is a compound having the general formula (XVII) or a pharmaceutically acceptable salt thereof,

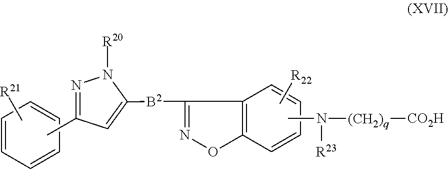

wherein:

R²³ is an alkyl group having 1 to 8 carbon atoms or an alkyl group having 1 to 8 carbon atoms and a halogen atom substituent;

q is an integer of 1 to 4;

R²⁰ is an alkyl group having 1 to 8 carbon atoms;

B² is an alkylene chain having 2 to 4 carbon atoms;

each of R²¹ and R²² is independently H, an alkyl group having 1 to 8 carbon atoms, a halogen atom, an alkyl group having 1 to 8 carbon atoms and a halogen atom substituent;

In one embodiment, the compounds having the formula (XVII), wherein N(R²³)((CH₂)_{q}—CO₂H) is attached to the 6th position of benzisoxazole;

R²³ is a methyl group;

q is 1;

B² is an ethylene chain;

R²¹ is an alkyl group having 1 to 8 carbon atoms, a halogen atom or an alkyl group having 1 to 8 carbon atoms and a halogen atom substituent; and

R²² is H, an alkyl group having 1 to 8 carbon atoms or an alkyl group having 1 to 8 carbon atoms and a halogen atom substituent.

Also provided is an activator of peroxisome proliferator activated receptor δ which contains as an effective component a compound having the formula (XV), (XVI) or (XVII) or a salt thereof.

In another embodiment, a compound has the following general formula (XVIII) or a salt thereof:

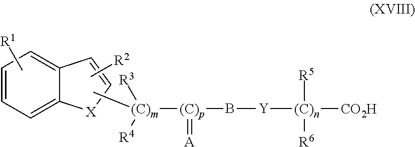

wherein:

R¹ represents hydrogen, halogen, hydroxyl, nitro, amino, cyano, carboxyl, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, a 5- or 6-membered heterocyclic group, an aralkyl group having an aryl moiety of 6-10 carbon atoms and an alkylene moiety of 1-8 carbon atoms, or an alkyl group having 1-8 carbon atoms and a 5- or 6-membered heterocyclic substituent;

R² represents hydrogen, an alkyl group having 1-8 carbon atoms, an alkenyl group having 2-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an acyl group having 2-8 carbon atoms, an aryl group having 6-10 carbon atoms, or an aralkyl group having an aryl moiety of 6-10 carbon atoms and an alkylene moiety of 1-8 carbon atoms;

each of R³, R⁴, R⁵ and R⁶ independently represents hydrogen, an alkyl group having 1-8 carbon atoms, or an alkyl group having 1-8 carbon atoms and having a halogen substituent;

X is oxygen, sulfur or NR⁷, R⁷ representing hydrogen, an alkyl group having 1-8 carbon atoms, an alkyl group having 1-8 carbon atoms and having a halogen substituent, an aralkyl group having an aryl moiety of 6-10 carbon atoms and an alkylene moiety of 1-8 carbon atoms, an acyl group having 2-8 carbon atoms, or an alkenyl group having 2-8 carbon atoms;

Y is oxygen, sulfur, NR⁸ or a bond, R⁸ representing hydrogen, an alkyl group having 1-8 carbon atoms, an alkyl group having 1-8 carbon atoms and having a halogen substituent, an acyl group having 2-8 carbon atoms, or an alkenyl group having 2-8 carbon atoms;

p is 0 or 1;

A is oxygen CH₂, N—NH₂ or N—OR⁹, R⁹ representing hydrogen, an alkyl group having 1-8 carbon atoms, an alkyl group having 1-8 carbon atoms and having a halogen substituent, an acyl group having 2-8 carbon atoms, an alkenyl group having 2-8 carbon atoms, or an aralkyl group having an aryl moiety of 6-10 carbon atoms and an alkylene moiety of 1-8 carbon atoms;

B represents, in the case of p=1, a benzene ring having or not having a substituent selected from the group consisting of halogen, hydroxyl, nitro, amino, an alkyl group having 1-8 carbon atoms, 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an alkoxy group having 1-8 carbon atoms and having a halogen substituent, an acyl group having 2-8 carbon atoms, an aryl group having 6-10 carbon atoms, or an aralkyl group having an aryl moiety of 6-10 carbon atoms and an alkylene moiety of 1-8 carbon atoms, and, in the case of p=0, a condensed ring selected from the group consisting of indole, benzofuran, benzisoxazole and 1,2-benzisothiazole, in which said condensed ring has or does not have a substituent selected from the group consisting of halogen, hydroxyl, nitro, amino, an alkyl group having 1-8 carbon atoms, 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an alkoxy group having 1-8 carbon atoms and having a halogen substituent, an acyl group having 2-8 carbon atoms, an aryl group having 6-10 carbon atoms, or an aralkyl group having an aryl moiety of 6-10 carbon atoms and an alkylene moiety of 1-8 carbon atoms;

Y is bonded to the benzene ring of B;

—(C(R³)(R⁴))ₘ— is bonded to the condensed ring of B at its 3-position;

m is an integer of 1 to 4;

n is an integer of 0 to 5; and

Y is a bond in the case of n=0.

In yet another embodiment, provided is a compound having the following formula (XIX) or a pharmacologically acceptable salt thereof:

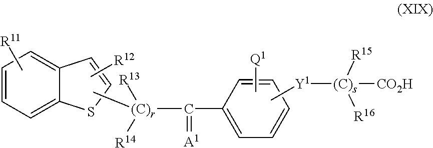

wherein:

R¹¹ represents hydrogen, halogen, hydroxyl, nitro, amino, cyano, carboxyl, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, a 5- or 6-membered heterocyclic group, an aralkyl group having an aryl moiety of 6-10 carbon atoms and an alkylene moiety of 1-8 carbon atoms, or an alkyl group having 1-8 carbon atoms and a 5- or 6-membered heterocyclic substituent,

R¹² represents hydrogen, an alkyl group having 1-8 carbon atoms, an alkenyl group having 2-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an acyl group having 2-8 carbon atoms, an aryl group having 6-10 carbon atoms, or an aralkyl group having an aryl moiety of 6-10 carbon atoms and an alkylene moiety of 1-8 carbon atoms,

each of R¹³, R¹⁴, R¹⁵ and R¹⁶ independently represents hydrogen, an alkyl group having 1-8 carbon atoms, or an alkyl group having 1-8 carbon atoms and having a halogen substituent,

Y¹ is oxygen, sulfur, NR¹⁸ or a bond, R¹⁸ representing hydrogen, an alkyl group having 1-8 carbon atoms, an alkyl group having 1-8 carbon atoms and having a halogen substituent, an acyl group having 2-8 carbon atoms, or an alkenyl group having 2-8 carbon atoms,

A¹ is oxygen, CH₂, N—NH₂ or N—OR¹⁹, R¹⁹ representing hydrogen, an alkyl group having 1-8 carbon atoms, an alkyl group having 1-8 carbon atoms and having a halogen substituent, an acyl group having 2-8 carbon atoms, an alkenyl group having 2-8 carbon atoms, or an aralkyl group having an aryl moiety of 6-10 carbon atoms and an alkylene moiety of 1-8 carbon atoms,

Q¹ represents hydrogen, halogen, hydroxyl, nitro, amino, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, or an aralkyl group having an aryl moiety of 6-10 carbon atoms and an alkylene moiety of 1-8 carbon atoms,

r is an integer of 1 to 4, and

s is an integer of 1 to 5.

In another embodiment, the compounds having the formula (XIX) wherein:

R¹¹ represents hydrogen, an alkyl group having 1-8 carbon atoms, or an alkyl group having 1-8 carbon atoms and having a halogen substituent;

R¹² represents an alkyl group having 1-8 carbon atoms or an alkyl group having 1-8 carbon atoms and having a halogen substituent;

each of R¹³ and R¹⁴ represents hydrogen;

each of R¹⁵ and R¹⁶ independently represents hydrogen or an alkyl group having 1-8 carbon atoms;

Y¹ is oxygen, N(alkyl having 1-8 carbon atoms), or represents an alkyl group having 1-8 carbon atoms, or a bond;

A¹ is oxygen, CH₂, N—OH, or N(O-benzyl);

Q¹ represents an alkyl group having 1-8 carbon atoms or an alkyl group having 1-8 carbon atoms and having a halogen substituent;

r is 2; and

s is 1 or 2.

In one embodiment, provided is a compound having the following formula (XX) or a pharmacologically acceptable salt thereof

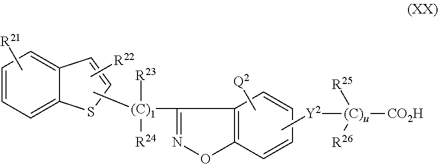

wherein:

R²¹ represents hydrogen, halogen, hydroxyl, nitro, amino, cyano, carboxyl, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, a 5- or 6-membered heterocyclic group, an aralkyl group having an aryl moiety of 6-10 carbon atoms and an alkylene moiety of 1-8 carbon atoms, or an alkyl group having 1-8 carbon atoms and a 5- or 6-membered heterocyclic substituent;

R²² represents hydrogen, an alkyl group having 1-8 carbon atoms, an alkenyl group having 2-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an acyl group having 2-8 carbon atoms, an aryl group having 6-10 carbon atoms, or an aralkyl group having an aryl moiety of 6-10 carbon atoms and an alkylene moiety of 1-8 carbon atoms;

each of R²³, R²⁴, R²⁵ and R²⁶ independently represents hydrogen, an alkyl group having 1-8 carbon atoms, or an alkyl group having 1-8 carbon atoms and having a halogen substituent;

Y² is oxygen, sulfur, NR²⁸ or a bond, R²⁸ representing hydrogen, an alkyl group having 1-8 carbon atoms, an alkyl group having 1-8 carbon atoms and having a halogen substituent, an acyl group having 2-8 carbon atoms, or an alkenyl group having 2-8 carbon atoms;

Q² represents hydrogen, halogen, hydroxyl, nitro, amino, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, or an aralkyl group having an aryl moiety of 6-10 carbon atoms and an alkylene moiety of 1-8 carbon atoms;

t is an integer of 1 to 4; and

u is an integer of 1 to 5.

In another embodiment, the compounds having formula (XX) wherein

R²¹ represents hydrogen, an alkyl group having 1-8 carbon atoms, or an alkyl group having 1-8 carbon atoms and having a halogen substituent;

R²² represents an alkyl group having 1-8 carbon atoms or an alkyl group having 1-8 carbon atoms and having a halogen substituent;

each of R²³ and R²⁴ represents hydrogen;

each of R²⁵ and R²⁶ independently represents hydrogen or an alkyl group having 1-8 carbon atoms;

Y² is oxygen, N(alkyl having 1-8 carbon atoms), or represents an alkyl group having 1-8 carbon atoms, or a bond;

Q² is an alkyl group having 1-8 carbon atoms or an alkyl group having 1-8 carbon atoms and having a halogen substituent;

t is 2; and

u is 1 or 2.

Also provided is an activator of peroxisome proliferator activated receptor which contains as an effective component a compound having the formula (XVIII), (XIX) or (XX) or a salt thereof.

### BRIEF DESCRIPTION OF THE DRAWINGS

FIG. 1 shows representative images of oxidative fibers following treatment.

FIG. 2 shows quantification of oxidative muscle fibers.

FIG. 3 shows body weight follow-up during regimen period. The results are expressed as mean±SEM.

FIG. 4 demonstrates screening according to fasted glycemia, plasma insulin and HOMA-IR. Results are expressed as mean±SEM.

FIG. 5A shows body weight follow-up during 4 weeks of treatment. Results are expressed as mean±SEM. An ANOVA two ways' test does not show an effect of time or treatment and an interaction between both. FIG. 5B shows body weight gain. Results are expressed as mean±SEM. An ANOVA two ways' test shows a significant effect of time and treatment without interaction between both. *: p<0.05 with a Bonferroni's post test analysis.

FIG. 6A shows plasma glucose levels after 17 days of treatment. FIG. 6B shows plasma insulin level after 17 days of treatment. FIG. 6C shows HOMA-IR after 17 days of treatment. FIG. 6D shows plasma adiponectine levels after 17 days of treatment. FIG. 6E shows plasma FFA after 17 days of treatment. FIG. 6F shows plasma TG levels after 17 days of treatment. Rats were fasted 4 hours before blood collection. Results are expressed as mean±SEM. *: p<0.05 with a paired t-test analysis.

FIG. 7A demonstrates the results of the oral glucose tolerance test after the oral glucose load (2.5 g/kg). FIG. 7B demonstrates the results of the oral glucose tolerance test as relative expression of glycaemia compared to the T0. Rats were fasted 6 hours before OGTT. Results are expressed as mean±SEM. An ANOVA two ways' test shows any significant effect of time and treatment and any interaction between both. *: p<0.05 ANOVA one way with a Dunnett's post test analysis.

FIG. 8 shows plasma insulin level after euglycemic clamp. Results are expressed as mean±SEM.

FIG. 9A shows glucose infusion rate during euglycemic clamp under 0.2 and 0.8 U/kg/h after 5 weeks of treatment that is a follow-up during the 210 minutes of infusion. An ANOVA two ways' test shows a significant effect of time and treatment without interaction between both. *: p<0.05 with a Bonferroni's post test analysis. FIG. 9B shows glucose infusion rate means during the 2 steady states. *: p<0.05 ANOVA one way with a Dunnett's post test analysis. After 6 hours of fasting, awaked rats were infused during 2 hours with 0.2 U/kg/h insulin and 0.8 U/kg/h insulin thereafter. GIR was readjusted every 10 minutes if necessary. Results are expressed as mean±SEM.

FIG. 10 shows glucose flux assessment during euglycaemic clamps under 0.2 U/kg/h insulin. Results are expressed as mean±SEM. *: p<0.05 ANOVA one way with a Dunnett's post test analysis.

FIG. 11 shows glucose flux assessment during euglycaemic clamps under 0.8 U/kg/h insulin. Results are expressed as mean±SEM. *: p<0.05 ANOVA one way with a Bonferroni's post test analysis.

FIG. 12A shows liver weight after 5 weeks of treatment. FIG. 12B shows white adipose tissue weight after 5 weeks of treatment. Rats were fasted 10 hours. Results are expressed as mean±SEM. *: p<0.05 ANOVA one way with a Dunnett's post test analysis.

FIG. 13 shows the content of triglycerides in liver after 5 weeks of treatment. Rats were fasted 10 hours. Results are expressed as mean±SEM.

FIG. 14 shows TNF-α content in liver and epididymal white adipose tissue after 5 weeks of treatment. Rats were fasted 10 hours. Results are expressed as mean±SEM. *: p<0.05 ANOVA one way with a Dunnett's post test analysis.

### DETAILED DESCRIPTION

### Definitions

Unless defined otherwise, all technical and scientific terms used herein have the same meaning as is commonly understood by one of ordinary skill in the art. In the event that there are a plurality of definitions for a term herein, those in this section prevail unless stated otherwise.

The singular forms “a,” “an,” and “the” include plural references, unless the context clearly dictates otherwise.

As used herein “subject” is an animal, such as a mammal, including human, such as a patient.

As used herein, biological activity refers to the in vivo activities of a compound or physiological responses that result upon in vivo administration of a compound, composition or other mixture. Biological activity, thus, encompasses therapeutic effects and pharmacokinetic behaviour of such compounds, compositions and mixtures. Biological activities can be observed in in vitro systems designed to test for such activities.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating inflammation.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, the IC₅₀ refers to an amount, concentration or dosage of a particular test compound that achieves a 50% inhibition of a maximal response in an assay that measures such response.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound. The instant disclosure is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, the nomenclature alkyl, alkoxy, carbonyl, etc. is used as is generally understood by those of skill in this art.

As used herein, alkyl, alkenyl and alkynyl carbon chains, if not specified, contain from 1 to 20 carbons, or 1 to 16 carbons, and are straight or branched. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, ethenyl, propenyl, butenyl, pentenyl, acetylenyl and hexynyl. As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “heteroalkyl” refers to a straight, branched or cyclic, in certain embodiments straight or branched, aliphatic hydrocarbon group having, inserted in the hydrocarbon chain one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl, S(═O)₂R′ or COR′, where R′ is alkyl, aryl, aralkyl, heteroaryl, heteroaralkyl, OY or —NYY′, where Y and Y′ are each independently hydrogen, alkyl, aryl, heteroaryl, cycloalkyl or heterocyclyl, in one embodiment having from 1 to about 20 atoms, in another embodiment having from 1 to 12 atoms in the chain.

As used herein, “cycloalkyl” refers to a saturated mono- or multicyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “substituted alkyl,” “substituted alkenyl,” “substituted alkynyl,” “substituted cycloalkyl,” “substituted cycloalkenyl,” and “substituted cycloalkynyl” refer to alkyl, alkenyl, alkynyl, cycloalkyl, cycloalkenyl and cycloalkynyl groups, respectively, that are substituted with one or more substituents, in certain embodiments one to three or four substituents, where the substituents are as defined herein, generally selected from Q1.

As used herein, “aryl” refers to aromatic monocyclic or multicyclic groups containing from 6 to 19 carbon atoms. Aryl groups include, but are not limited to groups such as fluorenyl, substituted fluorenyl, phenyl, substituted phenyl, naphthyl and substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including, but not limited to, nitrogen, oxygen or sulfur. The heteroaryl group may be optionally fused to a benzene ring. Heteroaryl groups include, but are not limited to, furyl, imidazolyl, pyrrolidinyl, pyrimidinyl, tetrazolyl, thienyl, pyridyl, pyrrolyl, N-methylpyrrolyl, quinolinyl and isoquinolinyl.

As used herein, a “heteroarylium” group is a heteroaryl group that is positively charged on one or more of the heteroatoms.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including, but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, amidino or the nitrogen may be quaternized to form an ammonium group where the substituents are selected as above.

As used herein, “substituted aryl,” “substituted heteroaryl” and “substituted heterocyclyl” refer to aryl, heteroaryl and heterocyclyl groups, respectively, that are substituted with one or more substituents, in certain embodiments one to three or four substituents, where the substituents are as defined herein, generally selected from Q1.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “alkylene” refers to a straight, branched or cyclic, in certain embodiments straight or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl, S(═O)₂R′ or COR′, where R′ is alkyl, aryl, aralkyl, heteroaryl, heteroaralkyl, —OY or —NYY′, where Y and Y′ are each independently hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “alkenylene” refers to a straight, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

As used herein, “alk(en)(yn)ylene” refers to a straight, branched or cyclic, in certain embodiments straight or branched, divalent aliphatic hydrocarbon group, in one embodiment having from 2 to about 20 carbon atoms and at least one triple bond, and at least one double bond; in another embodiment 1 to 12 carbons. In further embodiments, alk(en)(yn)ylene includes lower alk(en)(yn)ylene. There may be optionally inserted along the alkynylene group one or more oxygen, sulfur or substituted or unsubstituted nitrogen atoms, where the nitrogen substituent is alkyl. Alk(en)(yn)ylene groups include, but are not limited to, —C═C—(C₁₋₁₂)ₙ—C≡C—, where n is 1 or 2. The term “lower alk(en)(yn)ylene” refers to alk(en)(yn)ylene groups having up to 6 carbons. In certain embodiments, alk(en)(yn)ylene groups have about 4 carbon atoms.

As used herein, “cycloalkylene” refers to a divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic ring systems that respectively include at least one double bond and at least one triple bond. Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms. The ring systems of the cycloalkylene, cycloalkenylene and cycloalkynylene groups may be composed of one ring or two or more rings which may be joined together in a fused, bridged or spiro-connected fashion. “Cycloalk(en)(yn)ylene” refers to a cycloalkylene group containing at least one double bond and at least one triple bond.

As used herein, “substituted alkylene,” “substituted alkenylene,” “substituted alkynylene,” “substituted cycloalkylene,” “substituted cycloalkenylene,” and “substituted cycloalkynylene” refer to alkylene, alkenylene, alkynylene, cycloalkylene, cycloalkenylene and cycloalkynylene groups, respectively, that are substituted with one or more substituents, in certain embodiments one to three or four substituents, where the substituents are as defined herein, generally selected from Q¹.

As used herein, “arylene” refers to a monocyclic or polycyclic, in certain embodiments monocyclic, divalent aromatic group, in one embodiment having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. The term “lower arylene” refers to arylene groups having 5 or 6 carbons.

As used herein, “heteroarylene” refers to a divalent monocyclic or multicyclic aromatic ring system, in one embodiment of about 5 to about 15 members where one or more, in certain embodiments 1 to 3, of the atoms in the ring system is a heteroatom, that is, an element other than carbon, including, but not limited to, nitrogen, oxygen or sulfur.

As used herein, “heterocyclylene” refers to a divalent monocyclic or multicyclic non-aromatic ring system, in certain embodiments of 3 to 10 members, in one embodiment 4 to 7 members, in another embodiment 5 to 6 members, where one or more, including 1 to 3, of the atoms in the ring system is a heteroatom, that is, an element other than carbon, including, but not limited to, nitrogen, oxygen or sulfur.

As used herein, “substituted arylene,” “substituted heteroarylene” and “substituted heterocyclylene” refer to arylene, heteroarylene and heterocyclylene groups, respectively, that are substituted with one or more substituents, in certain embodiments one to three or four substituents, where the substituents are as defined herein, generally selected from Q¹.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyano, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1 chloro 2 fluoroethyl.

As used herein, “haloalkoxy” refers to RO in which R is a haloalkyl group.

As used herein, “carboxy” refers to a divalent radical, —C(O)O—.

As used herein, “aminocarbonyl” refers to C(O)NH₂.

As used herein, “alkylaminocarbonyl” refers to C(O)NHR in which R is alkyl, including lower alkyl. As used herein, “dialkylaminocarbonyl” refers to C(O)NR′R in which R′ and R are independently alkyl, including lower alkyl; “carboxamide” refers to groups of formula —NR′COR in which R′ and R are independently alkyl, including lower alkyl.

As used herein, “arylalkylaminocarbonyl” refers to —C(O)NRR′ in which one of R and R′ is aryl, including lower aryl, such as phenyl, and the other of R and R′ is alkyl, including lower alkyl.

As used herein, “arylaminocarbonyl” refers to —C(O)NHR in which R is aryl, including lower aryl, such as phenyl.

As used herein, “hydroxycarbonyl” refers to —COOH.

As used herein, “alkoxycarbonyl” refers to —C(O)OR in which R is alkyl, including lower alkyl.

As used herein, “aryloxycarbonyl” refers to —C(O)OR in which R is aryl, including lower aryl, such as phenyl.

As used herein, “alkoxy” and “alkylthio” refer to RO— and RS—, in which R is alkyl, including lower alkyl.

As used herein, “aryloxy” and “arylthio” refer to RO— and RS—, in which R is aryl, including lower aryl, such as phenyl.

Where the number of any given substituent is not specified (e.g., “haloalkyl”), there may be one or more substituents present. For example, “haloalkyl” may include one or more of the same or different halogens. As another example,

“C₁₋₃alkoxyphenyl” may include one or more of the same or different alkoxy groups containing one, two or three carbons.

As used herein, “selective PPARγ agonist” refers to a compound that is more active against PPARγ as compared to the compound's activity against PPARα and/or PPARγ. In certain embodiments, a selective PPARγ agonist is >100 times, >250 times, >500 times, >750 times, >1000 times or more active against PPARδ as compared to activity against PPARα and/or PPARγ.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

Compounds

Compounds of the Formula (I)

In the formula (I), examples of the alkyl groups having 1-8 carbon atoms include methyl, ethyl, propyl, isopropyl, butyl, isobutyl, t-butyl and pentyl.

Examples of the alkyl groups having 1-8 carbon atoms and a halogen substituent include methyl, ethyl, propyl, isopropyl, butyl, and t-butyl which are substituted with 1-3 halogens such as fluorine, chlorine, and bromine. Examples include trifluoromethyl, chloromethyl, 2-chloroethyl, 2-bromoethyl and 2-fluoroethyl.

Examples of the alkoxy groups having 1-8 carbon atoms include methoxy, ethoxy, propoxy, isopropoxy, butoxy, isobutoxy, t-butoxy and pentyloxy.

Examples of the alkoxy groups having 1-8 carbon atoms and a halogen substituent include methoxy, ethoxy, propoxy, isopropoxy, butoxy and t-butoxy groups substituted with 1-3 halogen atoms such as fluorine atom, chlorine atom or bromine atom. Trifluoromethoxy, chloromethoxy, 2-chloroethoxy, 2-bromoethoxy and 2-fluoroethoxy are included.

Examples of the alkenyl groups having 2-8 carbon atoms include vinyl and allyl.

Examples of the alkynyl groups having 2-8 carbon atoms include propargyl.

Examples of 3-7 membered cycloalkyl groups include cyclohexyl and cyclopentyl.

Examples of the alkyl groups having 1-8 carbon atoms and a 3-7 membered cycloalkyl substituent include cyclohexylmethyl and cyclopentylmethyl.

(1) In one embodiment, a compound provided is a compound of the formula (I) or salt thereof, in which R¹ is phenyl which can have substituents selected from the group consisting of C₁₋₈ alkyl, C₁₋₈ alkyl having 1-3 halogen atoms, C₁₋₈ alkoxy, C alkoxy having 1-3 halogen atoms, C₂₋₈ alkenyl, C₂₋₈ alkynyl, halogen, C₂₋₇ acyl, benzoyl, hydroxyl, nitro, amino, phenyl and pyridyl.

(2) In another embodiment, a compound provided is a compound of the formula (I), a salt thereof or (1), in which R² is C₂₋₈ alkyl.

(3) In another embodiment, a compound provided is a compound of the formula (I), a salt thereof, (1) or (2), in which R¹ is attached to the 2nd position. In the case that R¹ is attached to the 2nd position, R⁴ is attached to the 4th position and —X—Y— is attached to the 5th position, or R⁴ is attached to the 5th position and —X—Y— is attached to the 4th position.

(4) In another embodiment, a compound provided is a compound of the formula (I), a salt thereof, (1), (2) or (3), in which A is oxygen or sulfur.

(5) In another embodiment, a compound provided is a compound of the formula (I), a salt thereof, (1), (2), (3) or (4), in which X is a C₁₋₈ alkylene chain.

(6) In another embodiment, a compound provided is a compound of the formula (I), a salt thereof, (1), (2), (3), (4) or (5), in which Y is C(═=O).

(7) In another embodiment, a compound provided is a compound of the formula (I), a salt thereof, (1), (2), (3), (4), (5) or (6), in which each of R³, R⁴ and R⁵ is hydrogen, C₁₋₈ alkyl or C₁₋₈ alkyl having halogen.

(8) In another embodiment, a compound provided is a compound of the formula (I), a salt thereof, (1), (2), (3), (4), (5), (6) or (7), in which B is CH.

(9) In another embodiment, a compound provided is a compound of the formula (I), a salt thereof, (1), (2), (3), (4), (5), (6), (7) or (8), in which Z is oxygen.

(10) In another embodiment, a compound provided is a compound of the formula (I), a salt thereof, (1), (2), (3), (4), (5), (6), (7), (8) or (9), in which each of R⁶ and R⁷ is hydrogen or C₁₋₄ alkyl.

(11) In another embodiment, a compound provided is a compound of the formula (I), a salt thereof, (1), (2), (3), (4), (5), (6), (7), (8) or (9), in which R⁸ is hydrogen.

(12) In another embodiment, a compound provided is a compound of the formula (I) or a salt thereof, in which R¹ is phenyl or naphthyl, each of which can have substituents selected from the group consisting of C₁₋₈ alkyl, C₁₋₈ alkyl having halogen, C₁₋₈ alkoxy, C₁₋₈ alkoxy having halogen, C₂₋₈ alkenyl, C₂₋₈ alkynyl, halogen, C₂₋₇ acyl, benzoyl, hydroxyl, nitro, amino, phenyl and pyridyl;

R² is C₂₋₈ alkyl;

A is oxygen or sulfur;

X is a C₁₋₈ alkylene chain which can have a C₁₋₈ alkyl substituent and which can contain a double bond;

Y is C(═=O), CH═=CH, or C(═=CH₂);

each of R³, R⁴ and R⁵ is hydrogen, C₁₋₈ alkyl, C₁₋₈ alkyl having halogen, C₁₋₈ alkoxy, C₁₋₈ alkoxy having halogen, C₂₋₈ alkenyl, C₂₋₈ alkynyl, halogen, C₂₋₇ acyl, benzoyl, hydroxyl, nitro, amino, phenyl, or pyridyl;

B is CH;

Z is oxygen or sulfur;

each of R⁶ and R⁷ is hydrogen or C₁₋₈ alkyl; and

R⁸ is hydrogen or C₁₋₈ alkyl.

(13) In another embodiment, a compound provided is a compound of (12), in which X is a C₁₋₈ alkylene chain.

(14) In another embodiment, a compound provided is a compound of (12) or (13), in which R¹ is attached to the 2nd position.

(15) In another embodiment, a compound provided is a compound of (12), (13) or (14), in which R⁸ is hydrogen.

(16) In another embodiment, a compound provided is a compound of (12), (13), (14) or (15), in which the substituents of R³, R⁴ and R⁵ other than hydrogens are placed at ortho-positions with respect to —Z—CR⁶R⁷CO₂R⁸.

The compound of the formula (I) can be present in the form of geometrical isomers such as cis and trans and optical isomers. These isomers are included in the compounds provided. Further, the compounds provided can be in the form of pharmaceutically acceptable salts, such as alkali metal salts, e.g., sodium or potassium salt.

The non-limiting examples of the compounds of the formula (I) are:

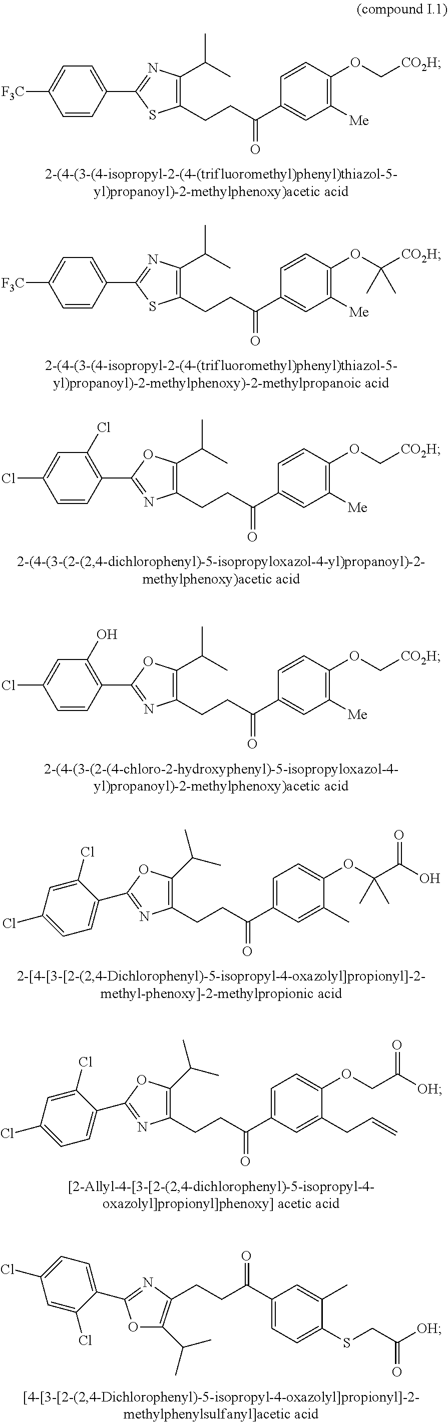

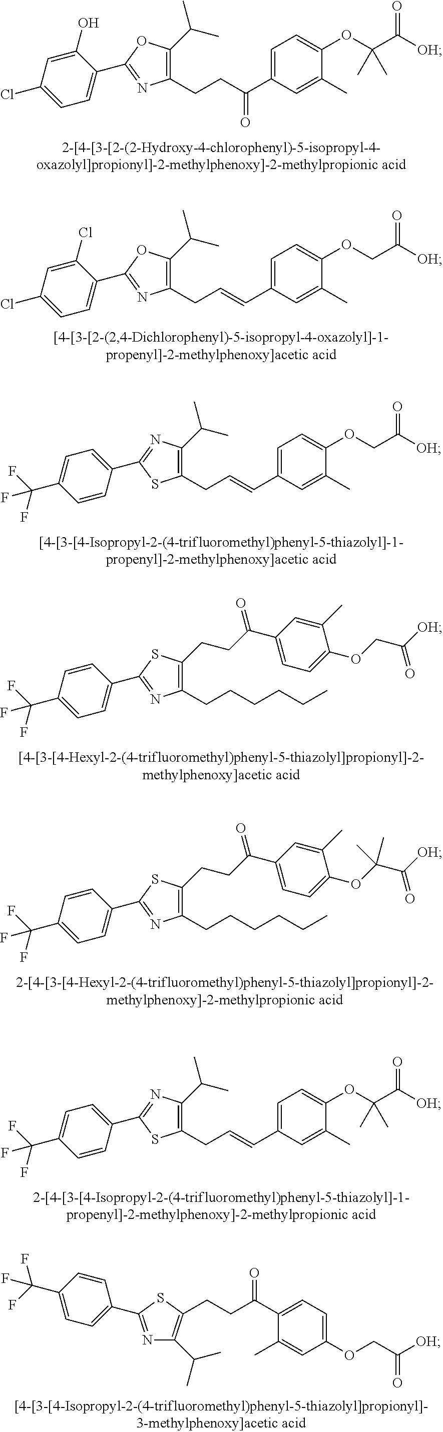

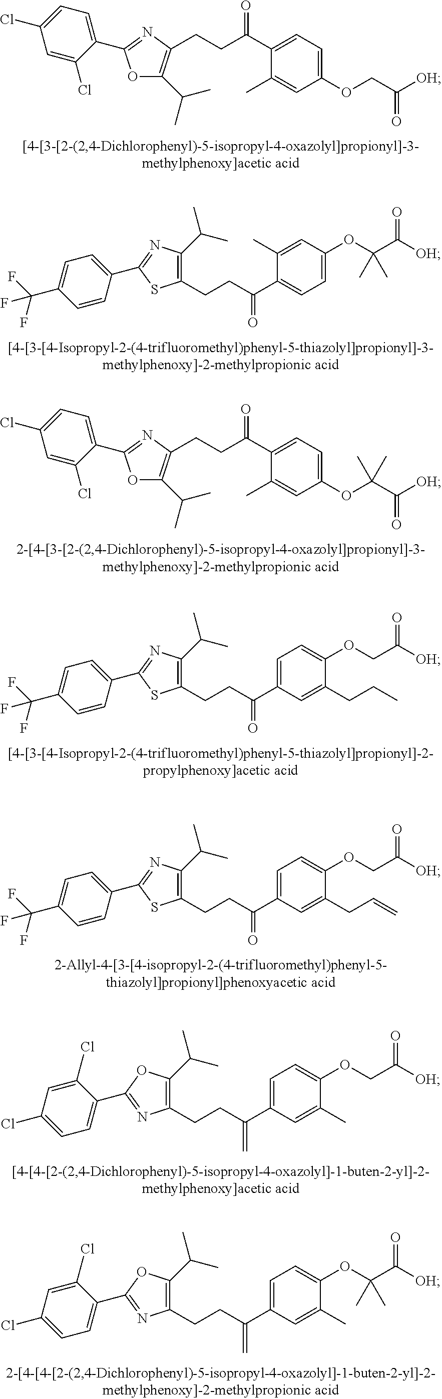

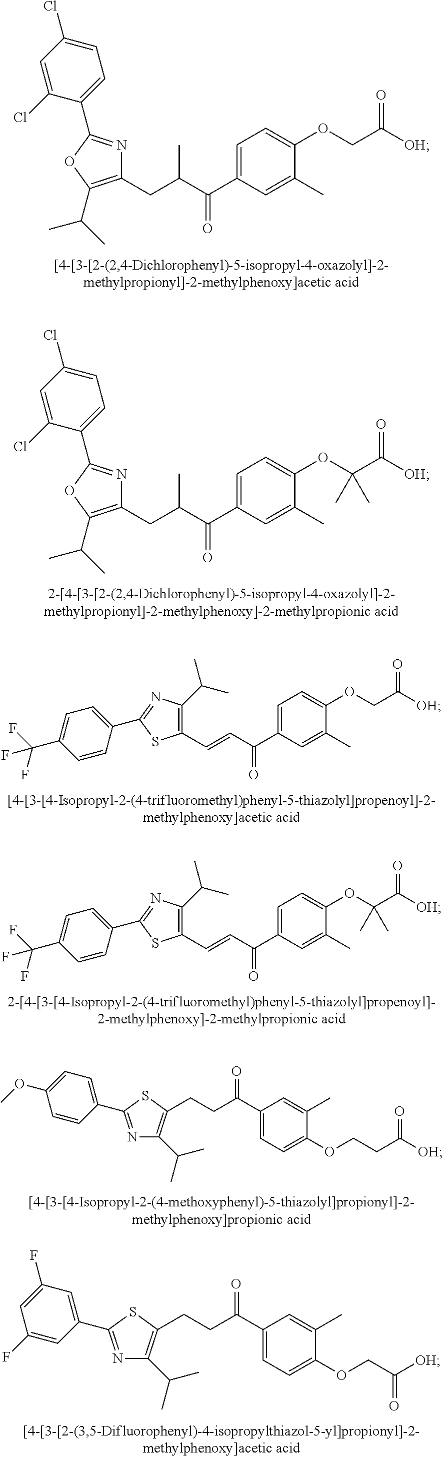

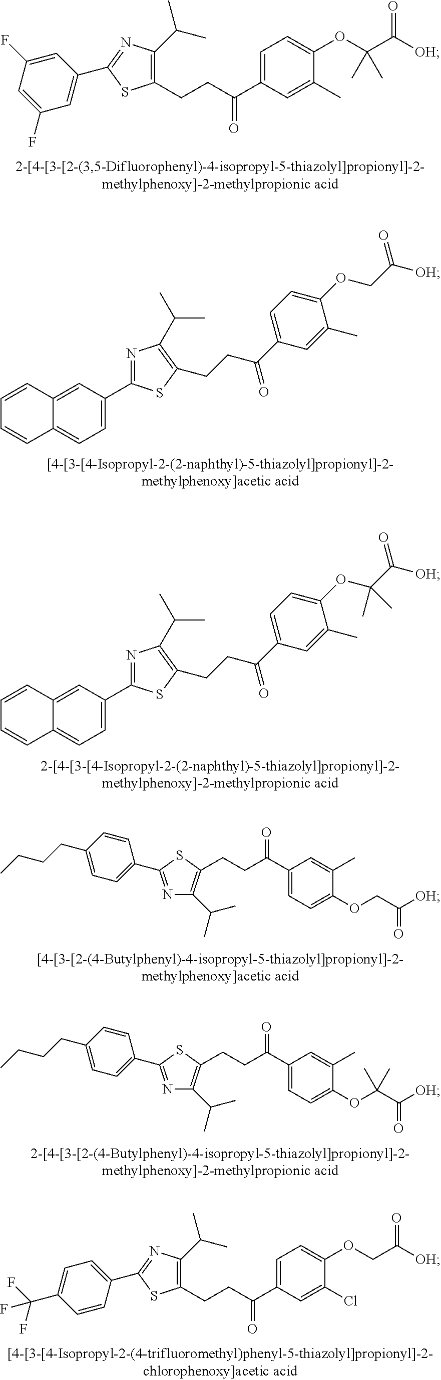

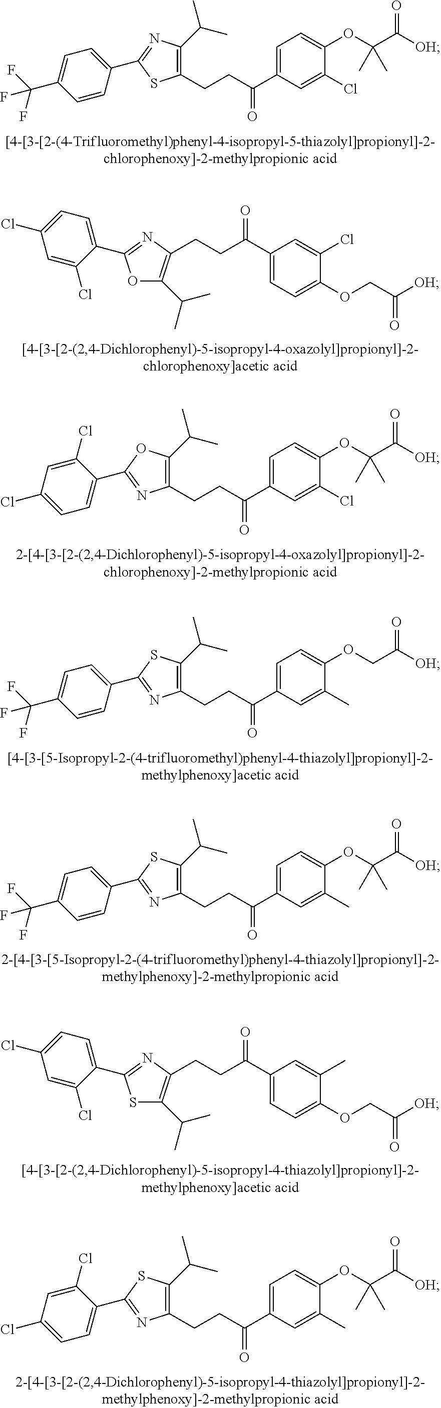

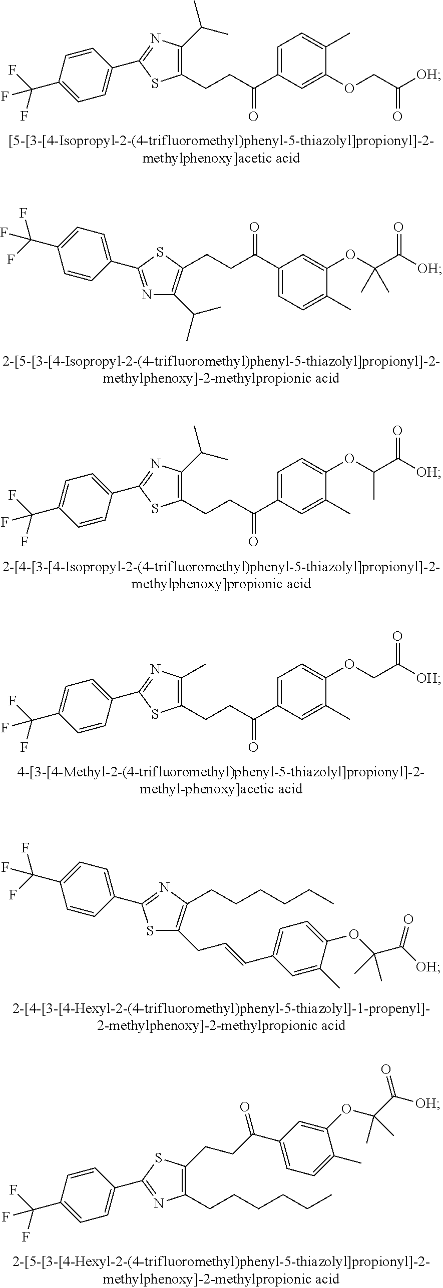

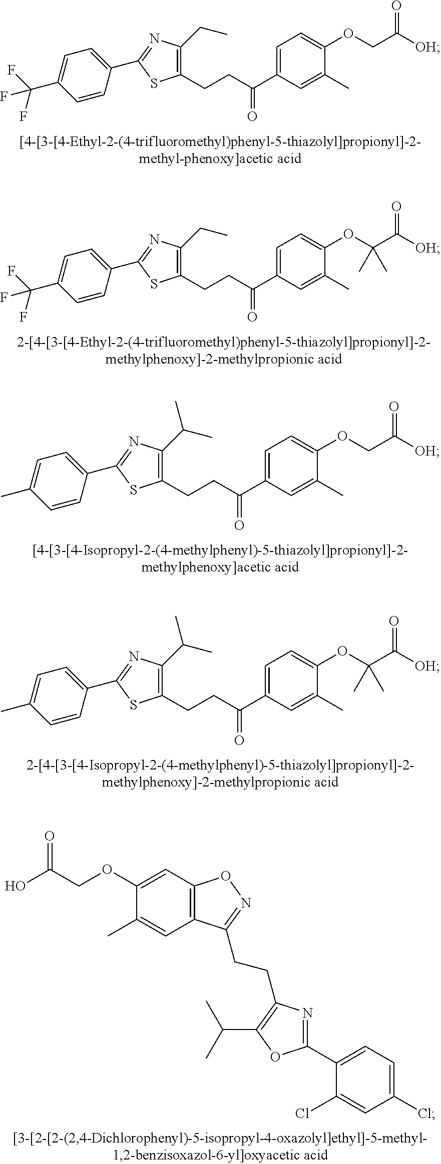

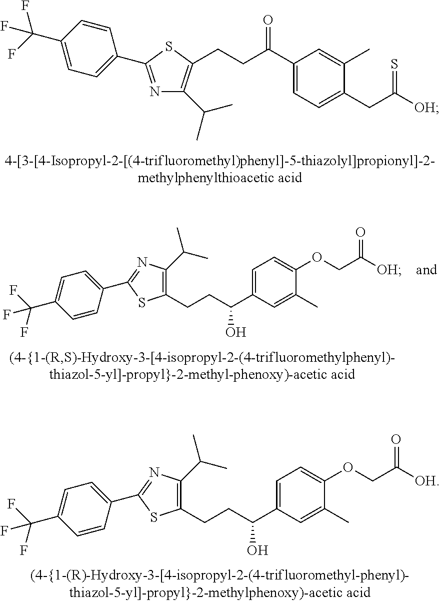

The processes for preparing the compound of the formula (I) provided herein are described below.

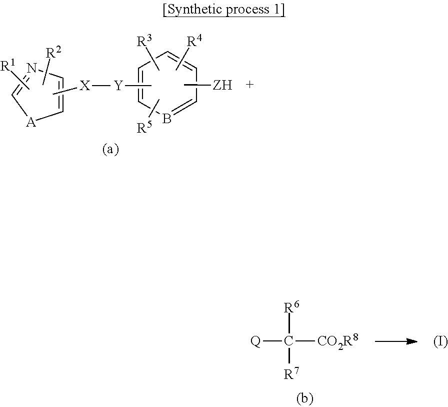

In the formulas, Q is a releasing group such as tosyloxy or halogen (e.g., bromine), and R¹, R², R³, R⁴, R⁵, R⁶, R⁷, R⁸, A, X, Y, B and Z are those described herein.

In the above-described process, the compound of the formula (I) according to the invention can be prepared by reacting a phenol or thiophenol compound of the general formula (a) with an acetic acid derivative of the general formula (b). The reaction can be carried out in a solvent such as methyl ethyl ketone in the presence of a base such as potassium carbonate.

The starting compound of the formula (a), can be prepared by a process similar to the below-mentioned synthetic scheme.

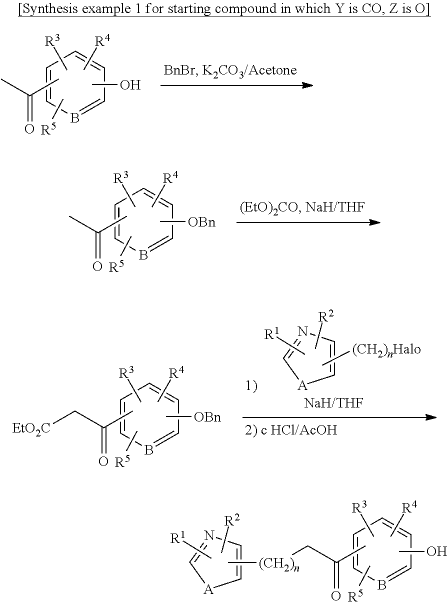

In the formulas, n is an integer of 1 to 7, Bn is benzyl, and R¹, R², R³, R⁴, R⁵, A and B are those described herein.

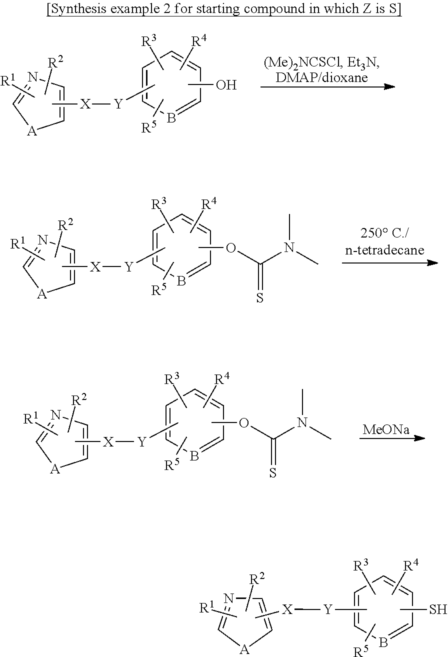

In the formulas, R¹, R², R³, R⁴, R⁵, A, B, X and Y are those described herein.

The phenol compound is treated with dimethylthiocarbamoyl chloride in the presence of a base such as triethylamine to obtain a dimethylthiocarbamoyloxy compound. The dimethylthiocarbamoyloxy compound is heated in n-tetradecane or no solvent to obtain a dimethylcarbamoylsulfanyl compound as a rearranged compound. The dimethylcarbamoyl group is treated with NaOH or MeONa to be converted to a thiophenol compound.

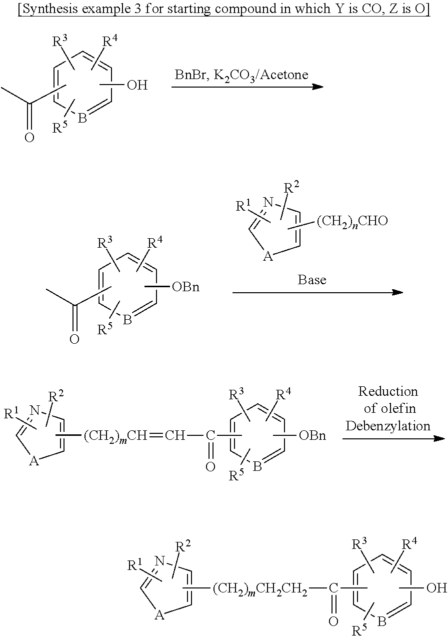

In the formulas, m is an integer of 0 to 6, and R¹, R², R³, R⁴, R⁵, A, B and Bn are those described herein.

The acetophenone compound and the aldehyde compound synthesized according to a conventional method are condensed with hydration using a base such as NaOH, KOH, MeONa, EtONa, piperidine in a solvent such as methanol, ethanol, anhydrous benzene to obtain a α,β-unsaturated ketone compound. The α,β-unsaturated ketone compound is treated, for example subjected to a hydride contact reduction to conduct reduction of the olefin and the debenzylation to obtain the subject compound.

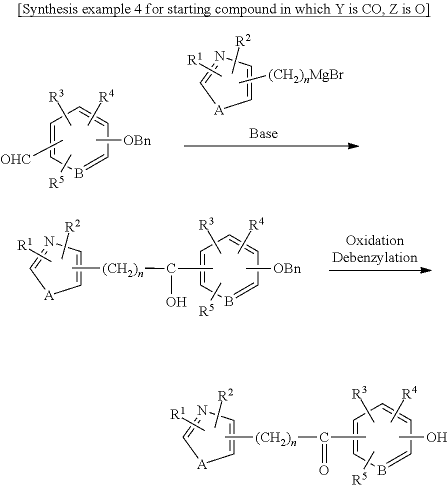

In the formulas, R¹, R², R³, R⁴, R⁵, A, B, n and Bn are those described herein.

The benzaldehyde compound is treated with a Grignard reagent obtained according to a conventional method in the presence of a solvent such as a ether or THF under the condition of a low temperature to obtain an alcohol compound. The alcohol compound can be converted into a ketone compound by using a Jones reagent (chromium(VI)oxide-sulfuric acid-acetone) or chromium(VI)-pyridine complex (e.g., pyridinium chlorochromate, pyridinium dichromate). The alcohol compound can also be converted into the ketone body in the same manner by using DMSO oxidation. Finally, the ketone body is subjected to debenzylation to be converted into the subject phenol compound.

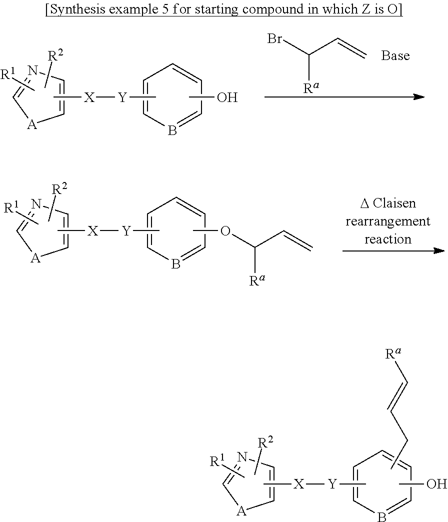

In the formulas, R^{a} is hydrogen atom or an alkyl group having 1 to 5 carbon atoms, and R¹, R², A, X, Y and B are those described herein.

The phenol compound is subjected to an allylation according to a conventional method, and heated (at 150° C. or higher) with no solvent or in a solvent such as quinoline to obtain a compound having the rearranged allyl group at the ortho-position.

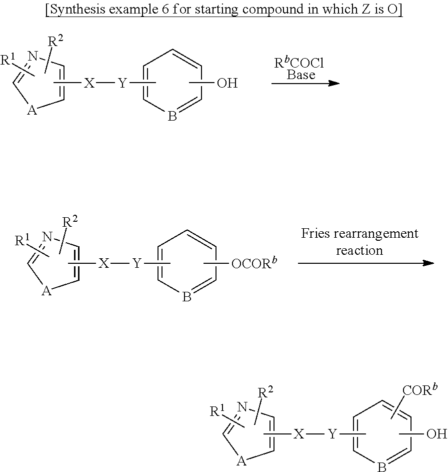

In the formulas, R^{b} is an alkyl group having 1 to 6 carbon atoms, and R¹, R², A, X, Y and B are those described herein.

The phenol compound is subjected to an acylation according to a conventional method, and heated in the presence of a Lewis acid catalyst to obtain a compound having the rearranged acyl group at the ortho-position.

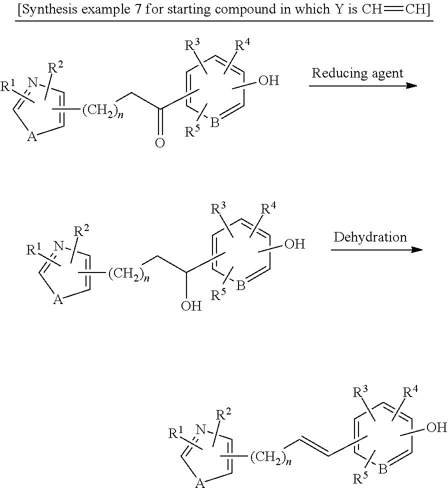

In the formulas, R¹, R², R³, R⁴, R⁵, A, B, n and Bn are those described herein.

The phenol compound obtained in the Synthesis example 1 for starting compound is treated with a reducing agent such as lithium aluminum hydride, sodium boron hydride to obtain an alcohol compound. The alcohol compound is subjected to dehydration using a halogenation agent, a sulfonation agent or a dehydration agent to obtain an olefin compound.

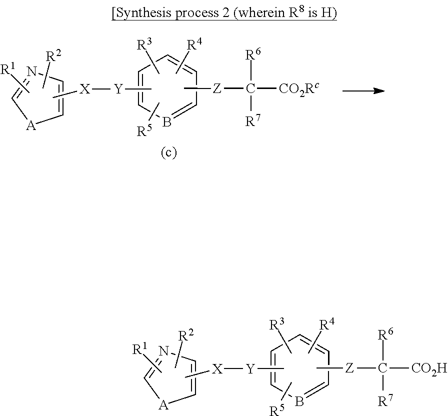

In the formulas, R^{c} is an alkyl group having 1 to 8 carbon atoms, and R¹, R², R³, R⁴, R⁵, R⁶, R⁷, A, X, Y, B and Z are those described herein.

In the above-illustrated process for preparation, a compound of the formula (I) (R⁸═=H) according to the invention can be obtained by the ester compound of the formula (c) is hydrolyzed in a solvent such as aqueous ethanol in the presence of a base such as sodium hydroxide, potassium hydroxide or lithium hydroxide.

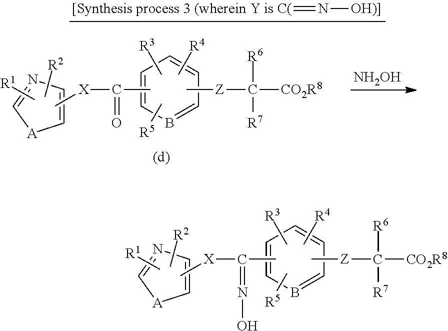

In the formulas, R¹, R², R³, R⁴, R⁵, R⁶, R⁷, A, X, B and Z are those described herein.

In the above-illustrated process, a compound of the formula (I) (Y is C(═=N—-OH)) according to the invention can be obtained by reacting the ketone compound of the formula (d) with hydroxylamine.

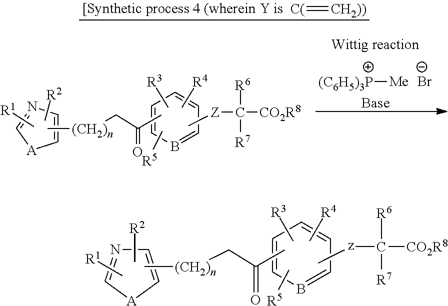

In the formulas, R¹, R², R³, R⁴, R⁵, R⁶, R⁷, A, B, Z and n are those described herein.

The ketone compound (Y is C(═=O)) can be treated with methyl triphenyl phosphonium bromide in the presence of a base such as t-BuOK, n-BuLi, sec-BuLi, EtONa in a solvent such as a dry ether or THE (according to Wittig reaction) to introduce a methylene chain into the compound.

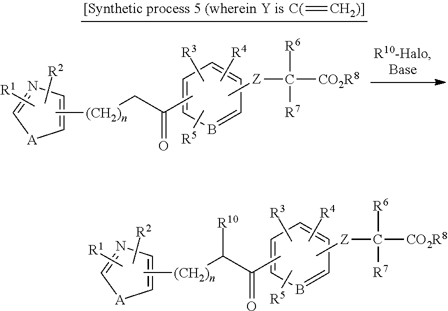

In the formulas, R¹⁰ is an alkyl group having 1 to 10 carbon atoms, R¹, R², R³, R⁴, R⁵, R⁶, R⁷, R⁸, A, B, Z and n are those described herein.

The ketone compound (Y is C(═=O)) can be treated with alkyl halide such as iodomethane in the presence of a base such as t-BuOK, BuLi, EtONa, NaH in a solvent such as a dry ether or THF to introduce an alkyl chain into the compound at the α-position of the carbonyl group.

Synthesis of an exemplary compound represented by formula (I):

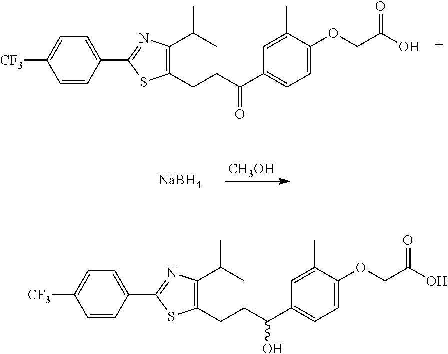

The S-stereoisomer is prepared as represented in the following scheme:

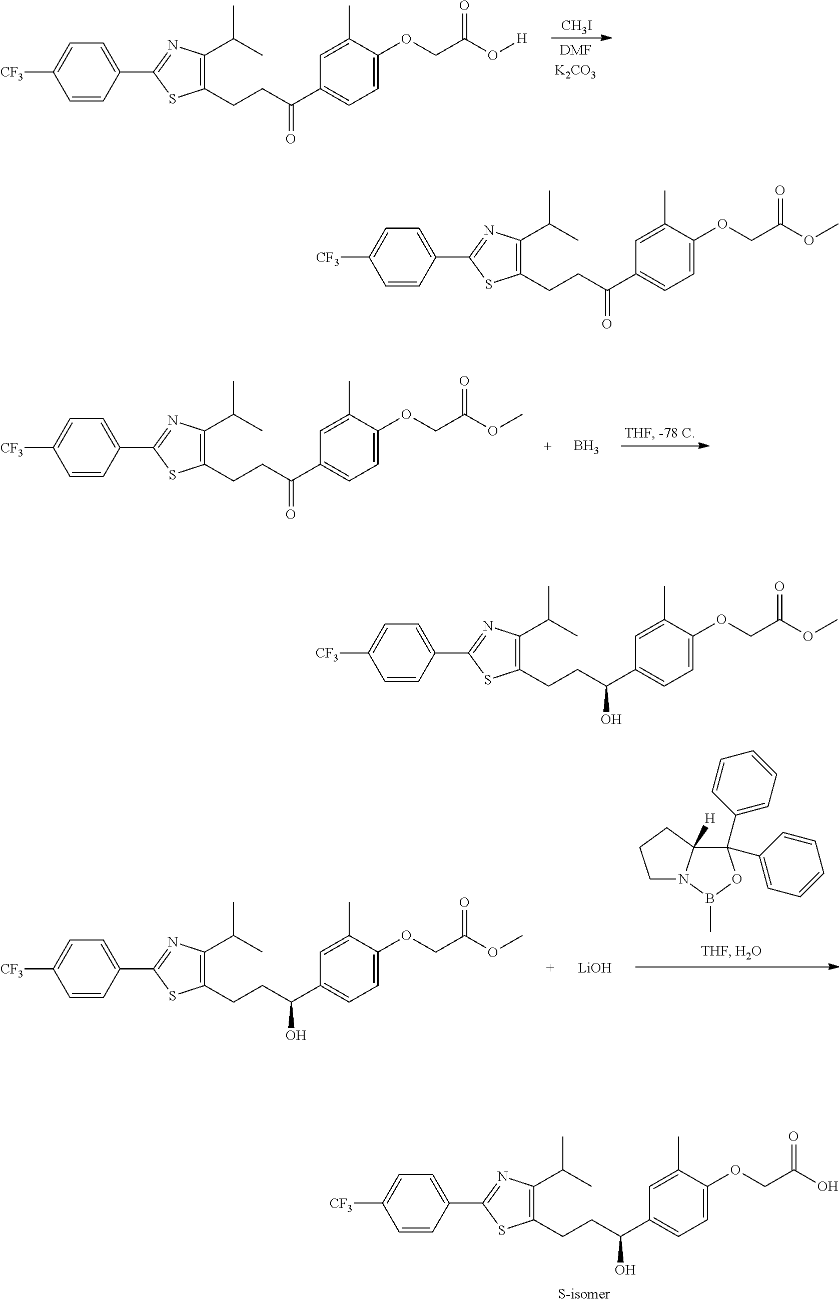

The R-stereoisomer is prepared as represented in the following scheme:

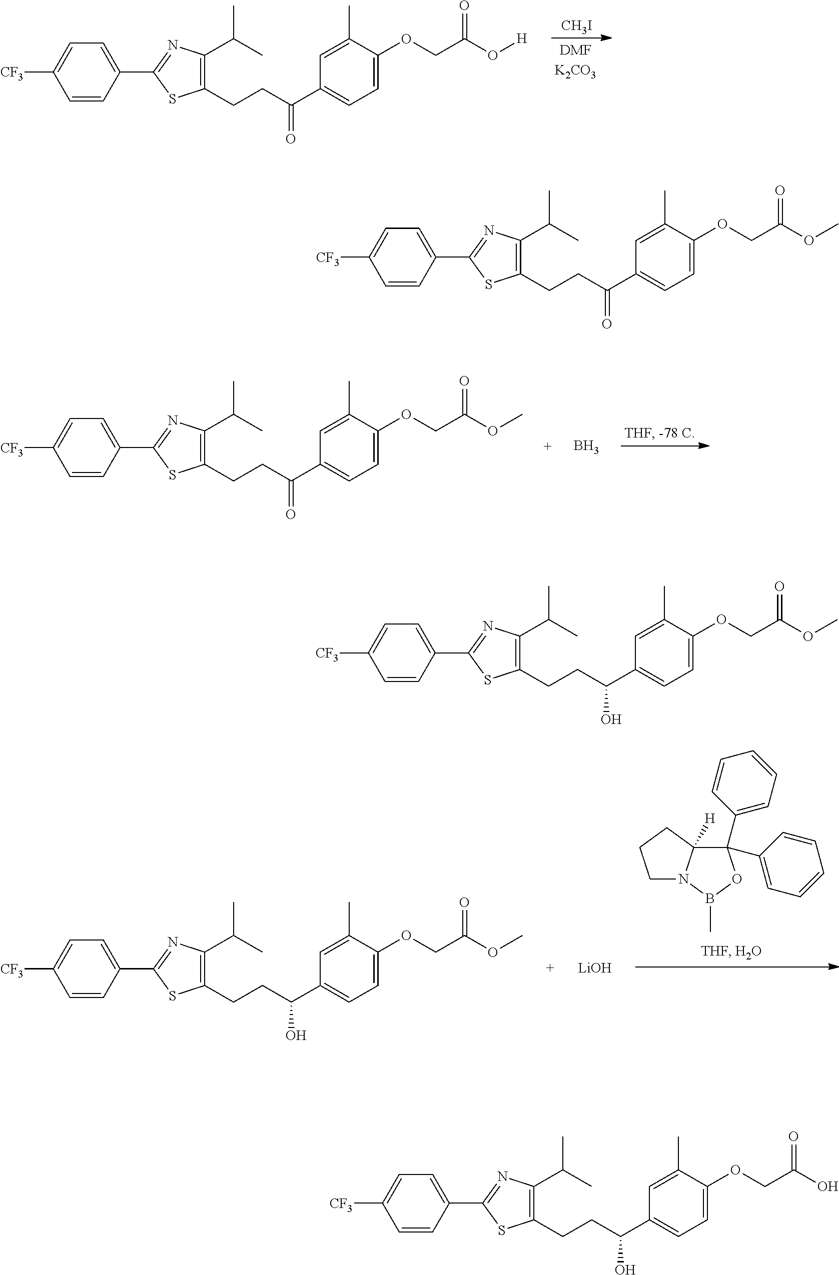

(I) In one embodiment, compounds for use in the methods provided herein have the following formula I:

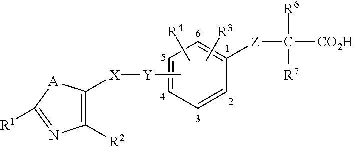

in which R¹, R², R³, R⁴, R⁶, R⁷, A, X, Y and Z are shown in Tables 1 to 4.

TABLE 1

| A | R¹ | R² | R³ | R⁴ | R⁶ | R⁷ | X | Y | Z |
|---|---|---|---|---|---|---|---|---|---|
| S | (4-CF₃)Ph | Isopropyl | Me(2) | H | H | H | CH₂CH₂ | C═O(4) | O |
| S | (4-CF₃)Ph | Isopropyl | Me(2) | H | H | H | CH₂CH₂ | C—OH(4) | O |
| S | (4-CF₃)Ph | Isopropyl | Me(2) | H | H | H | CH₂CH₂ | C—OH(4) | O |
| | | | | | | | | (R-isomer) | |
| S | (4-CF₃)Ph | Isopropyl | Me(2) | H | H | H | CH₂CH₂ | C—OH(4) | O |
| | | | | | | | | (S-isomer) | |
| S | (4-CF₃)Ph | Isopropyl | Me(2) | H | Me | Me | CH₂CH₂ | C═O(4) | O |
| S | (4-CF₃)Ph | Isopropyl | Me(2) | H | H | H | CH₂ | CH═CH(4) | O |
| S | (4-CF₃)Ph | Hexyl | Me(2) | H | H | H | CH₂CH₂ | C═O(4) | O |
| S | (4-CF₃)Ph | Hexyl | Me(2) | H | Me | Me | CH₂CH₂ | C═O(4) | O |
| S | (4-CF₃)Ph | Isopropyl | Me(2) | H | Me | Me | CH₂ | CH═CH(4) | O |
| S | (4-CF₃)Ph | Isopropyl | Me(3) | H | H | H | CH₂CH₂ | C═O(4) | O |
| S | (4-CF₃)Ph | Isopropyl | Me(3) | H | Me | Me | CH₂CH₂ | C═O(4) | O |
| S | (4-CF₃)Ph | Isopropyl | Pr(2) | H | H | H | CH₂CH₂ | C═O(4) | O |
| S | (4-CF₃)Ph | Isopropyl | Allyl(2) | H | H | H | CH₂CH₂ | C═O(4) | O |
| S | (4-CF₃)Ph | Isopropyl | Me(2) | H | H | H | CH═CH | C═O(4) | O |
| S | (4-CF₃)Ph | Isopropyl | Me(2) | H | Me | Me | CH═CH | C═O(4) | O |
| S | (4-OMe)Ph | Isopropyl | Me(2) | H | H | H | CH₂CH₂ | C═O(4) | O |
| S | (3,5,-F)Ph | Isopropyl | Me(2) | H | H | H | CH₂CH₂ | C═O(4) | O |
| S | (3,5,-F)Ph | Isopropyl | Me(2) | H | Me | Me | CH₂CH₂ | C═O(4) | O |
| S | 2-Naphthyl | Isopropyl | Me(2) | H | H | H | CH₂CH₂ | C═O(4) | O |
| S | 2-Naphthyl | Isopropyl | Me(2) | H | Me | Me | CH₂CH₂ | C═O(4) | O |
| S | (4-Bu)Ph | Isopropyl | Me(2) | H | H | H | CH₂CH₂ | C═O(4) | O |
| S | (4-Bu)Ph | Isopropyl | Me(2) | H | Me | Me | CH₂CH₂ | C═O(4) | O |
| S | (4-CF₃)Ph | Isopropyl | Cl(2) | H | H | H | CH₂CH₂ | C═O(4) | O |
| S | (4-CF₃)Ph | Isopropyl | Cl(2) | H | Me | Me | CH₂CH₂ | C═O(4) | O |
| S | (4-CF₃)Ph | Isopropyl | Me(2) | H | H | H | CH₂CH₂ | C═O(5) | O |
| S | (4-CF₃)Ph | Isopropyl | Me(2) | H | Me | Me | CH₂CH₂ | C═O(5) | O |
| S | (4-CF₃)Ph | Isopropyl | Me(2) | H | Me | H | CH₂CH₂ | C═O(4) | O |

Remark:
Numeral in ( ) means a position of the group.

TABLE 2

| A | R¹ | R² | R³ | R⁴ | R⁶ | R⁷ | X | Y | Z |
|---|---|---|---|---|---|---|---|---|---|
| S | (4-CF₃)Ph | Hexyl | Me(2) | H | Me | Me | CH₂ | CH═CH(4) | O |
| S | (4-CF₃)Ph | Hexyl | Me(2) | H | Me | Me | CH₂ | CH₂═CH₂(4) | O |
| S | (4-CF₃)Ph | Hexyl | Me(2) | H | Me | Me | CH₂CH₂ | C═O(5) | O |
| S | (4-CF₃)Ph | Ethyl | Me(2) | H | H | H | CH₂CH₂ | C═O(4) | O |
| S | (4-CF₃)Ph | Ethyl | Me(2) | H | Me | Me | CH₂CH₂ | C═O(4) | O |
| S | (4-Me)Ph | Isopropyl | Me(2) | H | H | H | CH₂CH₂ | C═O(4) | O |
| S | (4-Me)Ph | Isopropyl | Me(2) | H | Me | Me | CH₂CH₂ | C═O(4) | O |
| S | (4-CF₃)Ph | Isopropyl | Me(2) | H | H | H | CH₂CH₂ | C═O(4) | S |
| S | (4-Et)Ph | Isopropyl | Me(2) | H | H | H | CH₂CH₂ | C═O(4) | O |
| S | (4-iPr)Ph | Isopropyl | Me(2) | H | H | H | CH₂CH₂ | C═O(4) | O |
| S | (4-t-Bu)Ph | Isopropyl | Me(2) | H | H | H | CH₂CH₂ | C═O(4) | O |
| S | (4-Cl)Ph | Isopropyl | Me(2) | H | H | H | CH₂CH₂ | C═O(4) | O |
| S | (4-F)Ph | Isopropyl | Me(2) | H | H | H | CH₂CH₂ | C═O(4) | O |
| S | (4-NO₂)Ph | Isopropyl | Me(2) | H | H | H | CH₂CH₂ | C═O(4) | O |
| S | (4-NMe₂)Ph | Isopropyl | Me(2) | H | H | H | CH₂CH₂ | C═O(4) | O |
| S | (4-CF₃)Ph | Isopropyl | Me(2) | H | Me | Me | CH₂CH₂ | C═O(4) | O |
| S | (4-Et)Ph | Isopropyl | Me(2) | H | Me | Me | CH₂CH₂ | C═O(4) | O |
| S | (4-iPr)Ph | Isopropyl | Me(2) | H | Me | Me | CH₂CH₂ | C═O(4) | O |
| S | (4-t-Bu)Ph | Isopropyl | Me(2) | H | Me | Me | CH₂CH₂ | C═O(4) | O |
| S | (4-Cl)Ph | Isopropyl | Me(2) | H | Me | Me | CH₂CH₂ | C═O(4) | O |
| S | (4-F)Ph | Isopropyl | Me(2) | H | Me | Me | CH₂CH₂ | C═O(4) | O |
| S | (4-NO₂)Ph | Isopropyl | Me(2) | H | Me | Me | CH₂CH₂ | C═O(4) | O |
| S | (4-NMe₂)Ph | Isopropyl | Me(2) | H | Me | Me | CH₂CH₂ | C═O(4) | O |
| S | (4-Cl)Ph | Isopropyl | Allyl(2) | H | H | H | CH₂CH₂ | C═O(4) | O |

Remark:
Numeral in ( ) means a position of the group.

TABLE 3

| A | R¹ | R² | R³ | R⁴ | R⁶ | R⁷ | X | Y | Z |
|---|---|---|---|---|---|---|---|---|---|
| O | (2-OH,4-Cl)Ph | Isopropyl | Allyl(2) | H | H | H | CH₂CH₂ | C═O(4) | O |
| O | (2-OH,4-Cl)Ph | Isopropyl | Me(2) | H | Me | Me | CH₂CH₂ | CH═CH(3) | O |
| O | (4-Me)Ph | Isopropyl | Me(2) | H | H | H | CH₂CH₂ | C═O(4) | S |
| O | (2,4-Me)Ph | Isopropyl | Pr(2) | H | Me | Me | CH(Me)CH₂ | C═O(4) | O |
| S | (2-OH,4-Me)Ph | Bu | Benzyl(2) | H | H | H | CH₂CH₂ | C═O(3) | O |
| NH | (2-OH,4-CF₃)Ph | Pr | Acetyl(2) | H | H | H | CH₂CH₂ | C═O(4) | O |
| N—Me | (2-OH,4-Cl)Ph | Hexyl | Cl(2) | H | H | H | CH₂CH₂ | C═O(4) | O |
| S | (2,4-Me)Ph | Et | Br(2) | H | H | H | CH₂CH₂ | C═O(4) | S |
| S | (3,4-Cl)Ph | Bu | CF₃(2) | H | Me | Et | CH₂CH₂ | C═O(4) | O |
| S | (2,4-Me)Ph | Hexyl | Me(2) | Me(6) | Me | Me | CH(Me)CH₂ | C═O(4) | O |
| S | (2,4-Cl)Ph | Bu | Me(2) | Me(3) | H | H | CH₂CH₂ | C═O(4) | O |
| S | (2-OH,3,4-Me)Ph | Pr | Cl(2) | Cl(6) | H | H | CH₂CH₂ | CH═CH(4) | O |
| S | (2,4-F)Ph | Hexyl | Me(2) | H | Me | Me | CH₂CH₂ | CH═CH(4) | O |
| O | (3,4,5-Me)Ph | Et | Me(2) | H | H | H | CH₂CH₂ | C═O(4) | S |
| O | (2-OH,3,4-Me)Ph | Bu | Me(3) | H | Me | Me | CH₂CH₂ | C═O(4) | O |
| O | (2-OH,4-CF₃)Ph | Phenylethyl | Me(2,6) | H | H | H | CH₂CH₂ | C═O(3) | O |
| O | (4-OMe)Ph | Isopropyl | Me(2) | Me(6) | H | H | CH₂CH₂ | C═O(4) | O |
| S | (2-Cl,4-OPh)Ph | Isopropyl | Acetyl(2) | H | H | H | CH₂CH₂ | C═O(4) | O |
| NH | 1-Naphthyl | Isopropyl | Cl(3) | H | H | H | CH₂ | CH═CH(4) | S |
| N—Me | 2-Naphthyl | Isopropyl | Br(3) | H | Me | Et | CH(Me)CH₂ | C═O(4) | O |
| S | 2-Quinolyl | Isopropyl | CF₃(2) | H | Me | Me | CH₂CH₂ | C═O(4) | O |
| NH | 8-Quinolyl | Isopropyl | Me(2) | H | Me | Me | CH₂CH₂ | C═O(4) | O |
| N—Me | 3-Quinolyl | Isopropyl | Me(2) | H | H | H | CH₂CH₂ | C═O(4) | O |
| S | 2-Pryimidyl | Isopropyl | Allyl(3) | H | H | H | CH₂CH₂ | C═O(4) | O |

Remark:
Numeral in ( ) means a position of the group.

TABLE 4

| A | R¹ | R² | R³ | R⁴ | R⁶ | R⁷ | X | Y | Z |
|---|---|---|---|---|---|---|---|---|---|
| S | 2-Thyenyl | Isopropyl | Me(2) | H | H | H | CH₂ | CH═CH(4) | S |
| S | 2-Pyridyl | Isopropyl | Me(2) | H | H | H | CH₂CH₂ | C═O(4) | O |
| S | 4-Pyridyl | Isopropyl | Me(2) | H | H | H | CH₂CH₂ | C═O(4) | O |
| S | 5-Bt-2-Pyridyl | Isopropyl | Me(2) | H | H | H | CH₂CH₂ | C═O(4) | O |
| S | 5-Me-2-Pyridyl | Isopropyl | Me(2) | H | H | H | CH₂CH₂ | C═O(4) | O |
| S | 5-Et-2-Pyridyl | Isopropyl | Me(2) | H | Me | Me | CH₂CH₂ | C═O(4) | O |
| S | 2-Furanyl | Isopropyl | Me(2) | H | H | H | CH₂CH₂ | C═O(4) | O |
| S | 2-Imidazolyl | Isopropyl | Me(2) | H | Me | Et | CH₂CH₂ | C═O(4) | O |
| O | 2-Indolyl | Isopropyl | Pr(2) | H | Me | Me | CH₂CH₂ | C═O(4) | O |
| O | 2-Benzofuranyl | Isopropyl | Benzyl(2) | H | Me | Me | CH₂CH₂ | C═O(4) | O |
| O | 2-Benzothienyl | Isopropyl | Acetyl(2) | H | Me | Me | CH₂CH₂ | C═O(4) | S |
| O | 2-Benzoimidazolyl | Isopropyl | Cl(2) | Cl(6) | Me | Me | CH₂CH₂ | C═O(4) | S |
| S | (4-CF₃)Ph | sec-Bu | Me(2) | H | H | H | CH₂CH₂ | C═O(4) | O |
| S | (4-CF₃)Ph | sec-Bu | Me(2) | H | Me | Me | CH₂CH₂ | C═O(4) | O |
| S | (4-CF₃)Ph | Isobutyl | Me(2) | H | H | H | CH₂CH₂ | C═O(4) | O |
| S | (4-CF₃)Ph | Phenylethyl | Me(2) | H | H | H | CH₂CH₂ | C═O(4) | O |
| S | (4-CF₃)Ph | Isopropyl | CF₃(2) | H | H | H | CH₂CH₂ | C═O(4) | O |
| S | (4-CF₃)Ph | Isopropyl | CHF₂(2) | H | H | H | CH₂CH₂ | C═O(4) | O |
| S | (4-CF₃)Ph | Isopropyl | Me(2) | H | H | H | CH₂CH₂ | C═CH₂(4) | O |

Remark:
Numeral in ( ) means a position of the group.

(2) In another embodiment, compounds for use in the methods provided herein have the following formula I:

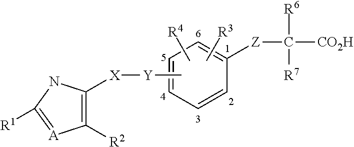

in which R¹, R², R³, R⁶, R⁷, A, X, Y and Z are shown in Tables 5 and 6.

TABLE 5

| A | R¹ | R² | R³ | R⁴ | R⁶ | R⁷ | X | Y | Z |
|---|---|---|---|---|---|---|---|---|---|
| O | (2,4-Cl)Ph | Isopropyl | Me(2) | H | Me | Me | CH₂CH₂ | C═O(4) | O |
| O | (2,4-Cl)Ph | Isopropyl | Me(2) | H | H | H | CH₂CH₂ | C═O(4) | O |
| O | (2,4-Cl)Ph | Isopropyl | Allyl(2) | H | H | H | CH₂CH₂ | C═O(4) | O |
| O | (2-OH,4-Cl)Ph | Isopropyl | Me(2) | H | H | H | CH₂CH₂ | C═O(4) | O |
| O | (2-OH,4-Cl)Ph | Isopropyl | Me(2) | H | Me | Me | CH₂CH₂ | C═O(4) | O |
| O | (2,4-Cl)Ph | Isopropyl | Me(2) | H | H | H | CH₂CH₂ | C═O(4) | S |
| O | (2,4-Cl)Ph | Isopropyl | Me(2) | H | H | H | CH₂CH₂ | CH═CH(4) | O |
| O | (2,4-Cl)Ph | Isopropyl | Me(3) | H | H | H | CH₂CH₂ | C═O(4) | O |
| O | (2,4-Cl)Ph | Isopropyl | Me(3) | H | Me | Me | CH₂CH₂ | C═O(4) | O |
| O | (2,4-Cl)Ph | Isopropyl | Me(2) | H | H | H | CH₂CH₂ | C═CH₂(4) | O |
| O | (2,4-Cl)Ph | Isopropyl | Me(2) | H | Me | Me | CH₂CH₂ | C═CH₂(4) | O |
| O | (2,4-Cl)Ph | Isopropyl | Me(2) | H | H | H | CH₂CH(Me) | C═O(4) | O |
| O | (2,4-Cl)Ph | Isopropyl | Me(2) | H | Me | Me | CH₂CH(Me) | C═O(4) | O |
| O | (2,4-Cl)Ph | Isopropyl | Cl(2) | H | H | H | CH₂CH₂ | C═O(4) | O |
| O | (2,4-Cl)Ph | Isopropyl | Cl(2) | H | Me | Me | CH₂CH₂ | C═O(4) | O |
| S | 4-CF₃)Ph | Isopropyl | Me(2) | H | H | H | CH₂CH₂ | C═O(4) | O |
| S | 4-CF₃)Ph | Isopropyl | Me(2) | H | Me | Me | CH₂CH₂ | C═O(4) | O |
| S | (2,4-Cl)Ph | Isopropyl | Me(2) | H | H | H | CH₂CH₂ | C═O(4) | O |
| S | (2,4-Cl)Ph | Isopropyl | Me(2) | H | Me | Me | CH₂CH₂ | C═O(4) | O |
| O | (2,4-Me)Ph | Isopropyl | Pr(3) | H | Me | Me | CH(Me)CH₂ | C═O(4) | O |
| S | (2-OH,4-Me)Ph | Bu | Benzyl(2) | H | H | H | CH₂CH₂ | C═O(3) | O |
| NH | (2-OH,4-CF₃)Ph | Pr | Acetyl(2) | H | H | H | CH₂CH₂ | C═O(4) | O |
| N—Me | (2-OH,4-Cl)Ph | Hexyl | Cl(2) | H | H | H | CH₂CH₂ | C═O(4) | O |
| S | (2,4-Me)Ph | Et | Br(2) | H | H | H | CH₂CH₂ | C═O(4) | S |
| O | (3,4-Cl)Ph | Bu | CF₃(3) | H | Me | Et | CH₂CH₂ | C═O(4) | O |

Remark:
Numeral in ( ) means a position of the group.

TABLE 6

| A | R¹ | R² | R³ | R⁴ | R⁶ | R⁷ | X | Y | Z |
|---|---|---|---|---|---|---|---|---|---|
| O | (2,4-Me)Ph | Hexyl | Me(2) | Me(6) | Me | Me | CH(Me)CH₂ | C═O(4) | O |
| O | (2,4-Cl)Ph | Bu | Me(2) | Me(3) | H | H | CH₂CH₂ | C═O(4) | O |
| O | (2-OH,3,4-Me)Ph | Pr | Allyl(2) | H | H | H | CH₂CH₂ | CH═CH(4) | O |
| S | (2,4-F)Ph | Hexyl | Ph(2) | H | Me | Me | CH₂CH₂ | CH═CH(4) | O |
| NH | (3,4,5-Me)Ph | Et | Me(2) | H | H | H | CH₂CH₂ | C═O(4) | S |
| N—Me | (2-OH,3,4-Me)Ph | Bu | Me(3) | H | Me | Me | CH₂CH₂ | C═O(4) | O |
| S | (2-OH,4-CF₃)Ph | Isopropyl | Me(2) | Me(6) | H | H | CH₂CH₂ | C═O(3) | O |
| O | (2-Cl,4-OMe)Ph | Isopropyl | Me(2) | Me(6) | H | H | CH₂CH₂ | C═O(4) | O |
| O | (2-Cl,4-OPh)Ph | Isopropyl | Acetyl(2) | H | H | H | CH₂CH₂ | C═O(4) | O |
| O | 1-Naphthyl | Isopropyl | Cl(2) | H | H | H | CH₂ | CH═CH(4) | S |
| O | 2-Naphthyl | Isopropyl | Br(2) | H | Me | Et | CH(Me)CH₂ | C═O(4) | O |
| S | 2-Quinolyl | Isopropyl | CF₃(2) | H | Me | Me | CH₂CH₂ | C═O(4) | O |
| NH | 8-Quinolyl | Isopropyl | Me(2) | H | Me | Me | CH₂CH₂ | C═O(4) | O |
| N—Me | 3-Quinolyl | Isopropyl | Me(2) | H | H | H | CH₂CH₂ | C═O(4) | O |
| S | 2-Pyrimidyl | Isopropyl | Allyl(2) | H | H | H | CH₂CH₂ | C═O(4) | O |
| O | 2-Thienyl | Isopropyl | Me(2) | H | H | H | CH₂ | CH═CH(4) | S |
| O | 2-Furanyl | Isopropyl | Me(2) | H | H | H | CH₂CH₂ | C═O(4) | O |
| O | 2-Imidazolyl | Isopropyl | Me(2) | H | Me | Et | CH₂CH₂ | C═O(4) | O |
| O | 2-Indolyl | Isopropyl | Pr(2) | H | Me | Me | CH₂CH₂ | C═O(4) | O |
| O | 2-Benzofuranyl | Isopropyl | Benzyl(2) | H | Me | Me | CH₂CH₂ | C═O(4) | O |
| S | 2-Benzothienyl | Isopropyl | Acetyl(2) | H | Me | Me | CH₂CH₂ | C═O(4) | S |
| S | 2-Benzimidazolyl | Isopropyl | Cl(2) | Cl(6) | Me | Me | CH₂CH₂ | C═O(4) | S |

Remark:
Numeral in ( ) means a position of the group.

3) In another embodiment, compounds for use in the methods provided herein have the following formula I:

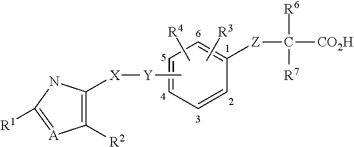

wherein R¹, R², R³, R⁴, R⁶, R⁷, A, X, Y and Z are shown in Table 7.

TABLE 7

| A | R¹ | R² | R³ | R⁴ | R⁶ | R⁷ | X | Y | Z |
|---|---|---|---|---|---|---|---|---|---|
| O | (2,4-Me)Ph | Hexyl | Me(2) | Me(6) | Me | Me | C══O(4) | CH(Me)CH₂ | O |
| O | (2,4-Cl)Ph | Bu | Me(2) | Me(3) | H | H | C══O(4) | CH₂CH₂ | O |
| S | (2-OH,4-CF₃)Ph | Isopr | Me(2) | Me(6) | H | H | C══O(3) | CH₂CH₂ | O |
| O | (2-Cl,4-OMe)Ph | Isopr | Me(2) | Me(6) | H | H | C══O(4) | CH₂CH₂ | O |
| S | 2-Benzimidazolyl | Isopr | Cl(2) | Cl(6) | Me | Me | C══O(4) | CH₂CH₂ | S |

Remark:
Numeral in ( ) means a position of the group.

Compounds of the Formula (II)

The variables in formula (II) are described in further detail below.

The halogen atom for R¹ and R² can be fluorine, chlorine, or bromine.

The alkyl groups having 1-8 carbon atoms for R¹, R², R³, R⁴ and R⁵ can be methyl, ethyl, propyl, isopropyl, butyl, isobutyl, t-butyl, or pentyl.

The alkoxy group having 1-8 carbon atoms for R¹ and R² can be methoxy, ethoxy, propyloxy, isopropyloxy, butyloxy, isobutyloxy, t-butyloxy, or pentyloxy.

The alkyl group having 1-8 carbon atoms which has 1-3 halogen substituents for R¹ and R² can be chloromethyl, fluoromethyl, bromomethyl, 2-chloroethyl, 2-fluoroethyl, or trifluoromethyl.

The alkoxy group having 1-8 carbon atoms which has 1-3 halogen substituents for R¹ and R² can be chloromethoxy, fluoromethoxy, bromomethoxy, 2-chloroethoxy, 2-fluoroethoxy, or trifluoroethoxy.

The alkenyl group having 2-8 carbon atoms for R¹ and R² can be vinyl or allyl. The alkynyl group having 2-8 carbon atoms can be propargyl. The cycloalkyl group having 3-7 carbon atoms can be cyclohexyl or cyclopentyl. The alkyl group having a 3-7 membered cycloalkyl substituent can be cyclohexylmethyl or cyclopentylmethyl.

The aryl group for the aryl group optionally having a substituent for R¹ and R² can be phenyl or naphthyl.

The arylalkyl group for the arylalkyl group (which has an aryl moiety of 6-10 carbon atoms and an alkyl moiety of 1-8 carbon atoms) optionally having a substituent can be benzyl or phenethyl.

The heterocyclic group for the heterocyclic group optionally having a substituent can be a 5-7 membered cyclic group having ring-forming 1-4 hetero atoms such as nitrogen, oxygen and sulfur. For instance, pyridyl, thienyl and furyl can be mentioned. Further, a benzene ring condensed with the heterocyclic group such as quinolyl or benzothienyl can be mentioned.

The heterocyclic group for the heterocyclic ring-alkyl group (the alkyl moiety has 1-8 carbon atoms) optionally having a substituent can be the same as that described hereinbefore for the heterocyclic group optionally having a substituent. The alkyl group preferably has 1-3 carbon atoms.

The substituent for the substituents of the aryl group optionally having a substituent, the arylalkyl group (the aryl moiety has 6-10 carbon atoms, and the alkyl moiety has 1-8 carbon atoms) optionally having a substituent, the heterocyclic group optionally having a substituent, and a heterocyclic ring-alkyl group (the alkyl moiety has 1-8 carbon atoms) optionally having a substituent can be a halogen atom such as chlorine, bromine, or fluorine, nitro, hydroxyl, amino, an alkyl amino group having 1-8 carbon atoms such as methylamino, or ethylamino, a dialkylamino group having 2-10 carbon atoms such as dimethylamino, an alkyl group having 1-8 carbon atoms such as methyl, ethyl, propyl, isopropyl, or butyl, an alkoxy group having 1-8 carbon atoms such as methoxy, ethoxy, propoxy, isopropoxy, or butoxy, an alkyl group having 1-8 carbon atoms which has 1-3 halogen substituents such as chloromethyl, fluoromethyl, bromomethyl, 2-chloroethyl, 2-fluoroethyl, or trifluoromethyl, an alkoxy group having 1-8 carbon atoms which has 1-3 halogen substituents such as chloromethoxy, fluoromethoxy, bromomethoxy, 2-chloroethoxy, 2-fluoroethoxy, or trifluoromethoxy, an alkyenyl group having 2-8 carbon atoms such as vinyl or allyl, an alkynyl group having 2-8 carbon atoms such as propargyl, a cycloalkyl group having 3-7 carbon atoms such as cyclohexyl or cyclopentyl, an alkyl group having a cycloalkyl group of 3-7 carbon atoms such as cyclohexylmethyl or cyclopentylmethyl, phenyl, or pyridyl.

The non-limiting examples of the compounds of the formula (II) are:

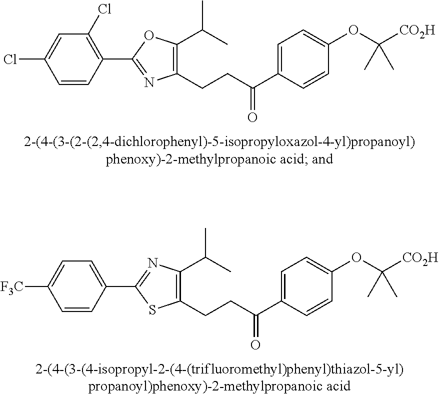

Compounds of the Formula (III)

The variables in formula (III) are described in further detail below.

The halogen atom, alkoxy groups having 1-8 carbon atoms, alkyl group having 1-8 carbon atoms which has 1-3 halogen substituents, alkoxy group having 1-8 carbon atoms which has 1-3 halogen substituents, alkenyl group having 2-8 carbon atoms, alkynyl group having 2-8 carbon atoms, cycloalkyl group having 3-7 carbon atoms, alkyl group having 1-8 carbon atoms which has a cycloalkyl group of 3-7 carbon atoms for R¹¹ and R¹² can be those described for the halogen atom, alkoxy group, alkyl group having 1-8 carbon atoms which has a halogen substituent, alkoxy group having 1-8 carbon atoms which has a halogen substituent, alkenyl, alkynyl, cycloalkyl group, and alkyl group having 1-8 carbon atoms which has a cycloalkyl group of 3-7 carbon atoms for R¹ and R².

The alkyl group having 1-8 carbon atoms for R¹¹, R¹², R¹⁴, and R¹⁵ can be an alkyl group described for R¹, R², R³, R⁴ and R⁵.

In the case that R¹¹ or R¹² is phenyl, naphthyl, benzyl, phenethyl, pyridyl, thienyl, furyl, quinolyl, or benzothienyl, these rings may have such substituents as a halogen atom such as chlorine, bromine, or fluorine, nitro, hydroxyl, amino, an alkyl amino group having 1-8 carbon atoms such as methylamino, or ethylamino, a dialkylamino group having 2 10 carbon atoms such as dimethylamino, an alkyl group having 1-8 carbon atoms such as methyl, ethyl, propyl, isopropyl, or butyl, an alkoxy group having 1-8 carbon atoms such as methoxy, ethoxy, propoxy, isopropoxy, or butoxy, an alkyl group having 1-8 carbon atoms which has 1-3 halogen substituents such as chloromethyl, fluoromethyl, bromomethyl, 2-chloroethyl, 2-fluoroethyl, or trifluoromethyl, an alkoxy group having 1-8 carbon atoms which has 1-3 halogen substituents such as chloromethoxy, fluoromethoxy, bromomethoxy, 2-chloroethoxy, 2-fluoroethoxy, or trifluoromethoxy, an alkyenyl group having 2-8 carbon atoms such as vinyl or allyl, an alkynyl group having 2-8 carbon atoms such as propargyl, a cycloalkyl group having 3-7 carbon atoms such as cyclohexyl or cyclopentyl, an alkyl group having a cycloalkyl group of 3-7 carbon atoms such as cyclohexylmethyl or cyclopentylmethyl, phenyl, or pyridyl.

(1) In one embodiment, the compound provided is a phenylacetic acid derivative of the formula (III) in which —X¹—Y¹—Z¹— is bonded to the 3- or 4-position of the phenylacetic acid or a salt thereof.

(2) In another embodiment, the compound provided is a phenylacetic acid derivative of the formula (III) or a phenylacetic acid derivative of (1) above in which X¹ is a bond, and Z¹ is —C(═=O)—, or a salt thereof.

(3) In another embodiment, the compound provided is a phenylacetic acid derivative of the formula (III) or a phenylacetic acid derivative of (1) or (2) above in which —X¹—Y¹—Z¹— is bonded to the 4-position of the oxazole ring, or a salt thereof.

(4) In another embodiment, the compound provided is a phenylacetic acid derivative of the formula (III) or a phenylacetic acid derivative of one of (1) to (3) above in which R¹¹ is a phenyl or naphthyl group which optionally has a substituent selected from the group consisting chlorine, fluorine, hydroxyl, an alkyl group having 1-5 carbon atoms, and an alkyl group having 1-5 carbon atoms, and it is bonded to the 2-position of the oxazole ring, or a salt thereof.

(5) In another embodiment, the compound provided is a phenylacetic acid derivative of the formula (III) or a phenylacetic acid derivative of one of (1) to (4) above in which R¹² is an alkyl group having 3-6 carbon atoms, and it is bonded to the 5-position of the oxazole ring, or a salt thereof.

The compound provided, that is a phenylacetic acid of the formula (III), or a salt thereof, can be a stereoisomer such as cis or trans, or an optical isomer. These isomers are included in the invention.

The compound provided includes a pharmaceutically acceptable salt such as an alkali metal salt, e.g., sodium salt or potassium salt. Further, the compounds provided can be in the form of pharmaceutically acceptable salts such as alkali metal salts, e.g., sodium salt and potassium salt.

The non-limiting examples of the compounds of the formula (III) are:

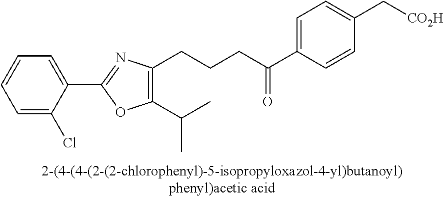

Compounds of the Formula (IV)

The variables in formula (IV) are described in further detail below.

In the formula (IV), R³, R⁴, R⁵, R⁶, R⁷, R⁸, R⁹, R¹⁰, the substituent of the alkylene chain of Y, the substituent of the aryl and the heterocyclic group of R³, the substituent of the alkyl group substituted with aryl of R², and the substituent of the alkyl group substituted with a heterocyclic group of R² can be an alkyl group having 1-8 carbon atoms. Examples of the alkyl groups include methyl, ethyl, propyl, isopropyl, butyl, isobutyl, t-butyl, pentyl and hexyl.

R² can be an alkyl group having 2-8 carbon atoms. Examples of the alkyl groups include ethyl, propyl, iso-propyl, butyl, isobutyl, t-butyl, pentyl and hexyl.

R², R⁴, R⁵, the substituent of the alkylene chain of Y, the substituent of the aryl or heterocyclic group of R¹, the substituent of the alkyl group substituted with aryl of R², and the substituent of the alkyl group substituted with a heterocyclic group of R² can be an alkyl groups having 1-8 carbon atoms substituted with 1-3 halogens. Examples of the haloalkyl groups include methyl, ethyl, propyl, isopropyl, butyl, and t-butyl which are substituted with 1-3 halogens such as fluorine, chlorine, and bromine. Trifluoromethyl, chloromethyl, 2-chloroethyl, 2-bromoethyl and 2-fluoroethyl are preferred.

R² and R³ can be an alkenyl group having 2-8 carbon atoms. Examples of the alkenyl groups include vinyl and allyl. R² and R³ can be an alkynyl group having 2-8 carbon atoms. Examples of the alkynyl groups include propargyl.

R³ can be a halogen atom. Examples of the halogen atoms include fluorine, chlorine and bromine.

R² can be a cycloalkyl group having 3-7 carbon atoms. Examples of the cycloalkyl groups include cyclopropyl, cyclopentyl and cyclohexyl.

The substituent of the aryl or heterocyclic group of R¹, the substituent of the alkyl group substituted with aryl of R², and the substituent of the alkyl group substituted with a heterocyclic group of R² can be an alkoxy groups having 1-8 carbon atoms. Examples of the alkoxy groups include methoxy, ethoxy, propoxy, isopropoxy, butoxy, isobutoxy, t-butoxy, pentyloxy and hexyloxy.

R¹ and the aryl moiety of the aryl substituted with alkyl of R² can be an aryl group. Examples of the aryl groups include phenyl and naphthyl. R¹ and the substituent of the alkyl group of R² can be a heterocyclic group having five to eight membered ring. Examples of the heterocyclic groups include pyridyl, thienyl, furyl, thiazolyl and quinolyl. R¹ can be a heterocyclic group having five to eight membered ring comprising one to three hetero atoms selected from the group consisting of nitrogen, oxygen and sulfur and the other atoms consisting of carbon. A benzene ring can be condensed with the heterocyclic ring. Examples of the condensed rings include quinoline ring and benzothiophene ring.

Y can be an alkylene chain having 1 to 8 carbon atoms. Examples of the alkylene chains include methylene and ethylene.

R³ can be one to three groups. Two or three groups of R³ can be different from each other.

R⁶ can be an alkyl group having 1-8 carbon atoms substituted with amino. Examples of the aminoalkyl groups include methyl, ethyl, propyl, isopropyl, butyl, isobutyl, t-butyl, pentyl and hexyl which are substituted with an amino group such as piperidino, pyrrolidino, dimethylamino, and diethylamino.

(1) In one embodiment, a compound provided is a compound of the formula (IV) or salt thereof, in which R¹ is attached to the 2nd position of the oxazole, thiazole or imidazole ring.

(2) In another embodiment, a compound provided is a compound of the formula (IV), a salt thereof or (1), in which B¹ is N, and B² is O.

(3) In another embodiment, a compound provided is a compound of the formula (IV), a salt thereof, (1) or (2), in which R⁶ is hydrogen.

(4) In another embodiment, a compound provided is a compound of the formula (IV), a salt thereof, (1), (2) or (3), in which X² is a bond.

(5) In another embodiment, a compound provided is a compound of the formula (IV), a salt thereof, (1), (2), (3) or (4), in which X¹ is a bond.

(6) In another embodiment, a compound provided is a compound of the formula (IV), a salt thereof, (1), (2), (3), (4) or (5), in which R¹ is aryl substituted with a group or atom selected from the group consisting of C₁₋₈ alkyl, C₁₋₈ alkoxy, C₁₋₈ alkyl substituted with 1-3 halogens, hydroxyl, nitro, amino, phenyl, pyridyl and halogen.

(7) In another embodiment, a compound provided is a compound of the formula (IV), a salt thereof, (1), (2), (3), (4), (5) or (6), in which R² is C₂₋₈ alkyl.

(8) In another embodiment, a compound provided is a compound of the formula (IV), a salt thereof, (1), (2), (3), (4), (5), (6) or (7), in which R³ is C₁₋₈ alkyl or C₂₋₈ alkenyl.

The compound of the formula (IV) can be in the form of pharmaceutically acceptable salts such as alkali metal salts, e.g., sodium salt and potassium salt.

The non-limiting examples of the compounds of the formula (IV) are:

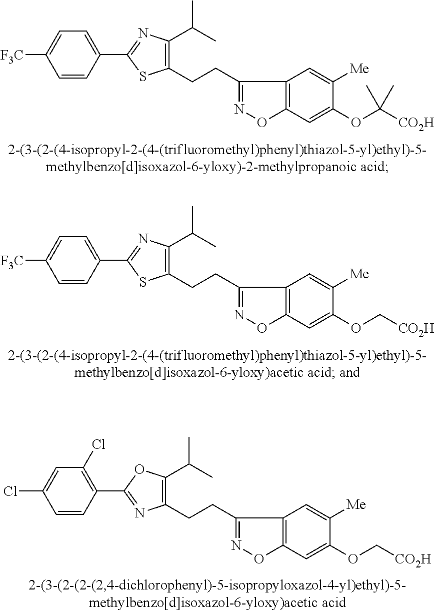

Compounds of the Formulae (V), (VI) and (VII)

Regarding the formula (V), examples of the alkyl groups having 1 to 8 carbon atoms which can be R¹, R³, R⁴, R⁵, R⁶, R⁷, the substituent of the 5-membered heterocyclic group for A, or the substituent of the alkylene chain having 2 to 6 carbon atoms for B include methyl, ethyl, propyl, isopropyl, butyl, i-butyl, t-butyl, pentyl or hexyl.

Examples of the alkenyl groups having 2 to 8 carbon atoms which can be R¹, R⁴, the substituent of the 5-membered heterocyclic group for A, or the substituent of the alkylene chain having 2 to 6 carbon atoms for B include vinyl and allyl.

Examples of the alkynyl groups having 2 to 8 carbon atoms which can be R¹, R⁴, the substituent of the 5-membered heterocyclic group for A, or the substituent of the alkylene chain having 2 to 6 carbon atoms for B include propargyl.

Examples of the 3- to 7-membered cycloalkyl groups which can be the substituent of the 5-membered heterocyclic group for A, or the substituent of the alkylene chain having 2 to 6 carbon atoms for B include cyclopropyl, cyclopentyl and cyclohexyl.

Examples of the alkoxy groups having 1 to 8 carbon atoms which can be R¹, R⁴, the substituent of the 5-membered heterocyclic group for A, or the substituent of the alkylene chain having 2 to 6 carbon atoms for B include methoxy, ethoxy, propoxy, isopropoxy, butoxy, i-butoxy, t-butoxy, pentyloxy and hexyloxy.

Examples of the halogen atoms which can be R¹, R⁴, or the substituent of the alkylene chain having 2 to 6 carbon atoms for B include fluorine, chlorine, and bromine.

Examples of the alkyl groups having 1 to 8 carbon atoms and a halogen atom substituent which can be R¹, R⁴, R⁵, R⁶, the substituent of the 5-membered heterocyclic group for A, or the substituent of the alkylene chain having 2 to 6 carbon atoms for B include methyl, ethyl, propyl, isopropyl, butyl and t-butyl which have substituents such as 1 to 3 fluorine, chlorine or bromine atoms. Preferred are trifluoromethyl, chloromethyl, 2-chloroethyl, 2-bromoethyl, and 2-fluoroethyl.

Examples of the alkoxy groups having 1 to 8 carbon atoms and a halogen atom substituent which can be R¹, R⁴, the substituent of the 5-membered heterocyclic group for A, or the substituent of the alkylene chain having 2 to 6 carbon atoms for B include methoxy, ethoxy, propoxy, isopropyloxy, butyloxy and t-butyloxy which have substituents such as 1 to 3 fluorine, chlorine or bromine atoms. Preferred are trifluoromethyloxy, chloromethyloxy, 2-chloroethyloxy, 2-bromoethyloxy, and 2-fluoroethyloxy.

Examples of the acyl groups having 2 to 8 carbon atoms which can be R¹ or R⁴, include acetyl and propionyl.

Examples of the aryl groups having 6 to 10 carbon atoms which can be R¹, R⁴, or the substituent of the 5-membered heterocyclic group for A, include phenyl.

Examples of the 5- or 6-membered heterocyclic groups which can be R¹, R⁴, or the substituent of the 5-membered heterocyclic group for A, include pyridyl.

Examples of the alkyl groups having 1 to 8 carbon atoms and a 3- to 7-cycloalkyl group substituent which can be the substituent of the 5-membered heterocyclic group for A, include methyl, ethyl, propyl, isopropyl, butyl, i-butyl, t-butyl, pentyl and hexyl which have cyclopropyl, cyclopentyl, or cyclophexyl substituent.

Examples of the aralkyl groups (which have an aryl moiety of 6 to 10 car-bon atoms and an alkylene moiety of 1 to 8 carbon atoms) which can be the substituent of the 5-membered heterocyclic group for A, include benzyl and phenethyl.

Examples of the alkyl groups having 1 to 8 carbon atoms and a 5- or 6-membered heterocyclic group which can be the substituent of the 5-membered heterocyclic group for A, include methyl, ethyl, propyl, isopropyl, butyl, i-butyl, t-butyl, pentyl and hexyl which have a pyridyl substituent.

Examples of the alkyl groups having 1 to 8 carbon atoms, alkenyl groups having 2 to 8 carbon atoms, alkynyl groups having 2 to 8 carbon atoms, alkoxy groups having 1 to 8 carbon atoms, halogen atoms, alkyl groups having 1 to 8 carbon atoms and a halogen atom substituent, alkoxy groups having 1 to 8 carbon atoms and a halogen atom substituent, acyl groups having 2 to 8 carbon atoms, aryl groups having 6 to 10 carbon atoms, and 5- or 6-membered heterocyclic groups which can be R¹¹ or R¹³ of the formula (VI) or R²¹ or R²³ of the formula (VII) are those described hereinabove for R¹ and R⁴ of the formula (V).

Examples of the alkyl groups having 1 to 8 carbon atoms, 3- to 7-membered cycloalkyl groups, alkenyl groups having 2 to 8 carbon atoms, alkynyl groups having 2 to 8 carbon atoms, alkoxy groups having 1 to 8 carbon atoms, alkyl groups having 1 to 8 carbon atoms and a 3- to 7-membered cycloalkyl group substituent, alkyl groups having 1 to 8 carbon atoms and a halogen atom substituent, alkoxy groups having 1 to 8 carbon atoms and a halogen atom substituent, aryl groups having 6 to 10 carbon atoms, 5- or 6-membered heterocyclic groups, aralkyl groups having an aryl moiety of 6 to 10 carbon atoms and an alkylene moiety of 1 to 8 carbon atoms, and alkyl groups having 1 to 8 carbon atoms and a 5- or 6-membered heterocyclic substituent which can be R¹² of the formula (VI) or R²² of the formula (VII) include those described hereinabove for the substituent of the 5-membered heterocyclic group for A of the formula (V).

Examples of the alkyl groups having 1 to 8 carbon atoms and alkyl groups having 1 to 8 carbon atoms and a halogen atom substituent which can be R¹⁴ or R¹⁵ of the formula (VI) or R²⁴ or R²⁵ of the formula (VII) include those described hereinabove for R⁵ and R⁶ of the formula (V).

R¹ in the formula (V), R¹¹ in the formula (VI), and R²¹ in the formula (VII) can be attached to the benzene ring or the like in a single or plural number (1 to 3). If each of R¹, R¹¹ and R²¹ is present in a plural number, the plural groups can be the same or different.

R⁴ in the formula (V), R¹³ in the formula (VI), and R²³ in the formula (VII) can be attached to the benzene ring or the like in a single or plural number (1 to 3). If each of R⁴, R¹³ and R²³ is present in a plural number, the plural groups can be the same or different.

The substituent group of the 5-membered heterocyclic group for A in the formula (V), R¹² in the formula (VI), and R²² in the formula (VII) can be attached to the heterocyclic ring in a single or plural number (1 or 2). If each of the substituent group of the 5-membered heterocyclic group for A, R¹² and R²² is present in plural number, the plural groups can be the same or different.

The preferred compounds are described below.

(1) In one embodiment, compounds of the formula (V) in which A is pyrazole, and salts thereof.

(2) In another embodiment, compounds of (1) above in which —(CH₂)ₙ— is attached the pyrazole at 1-position thereof, and salts thereof.

(3) In another embodiment, compounds of (1) above in which —(CH₂)ₙ— is attached the pyrazole at 3-position thereof, and salts thereof.

(4) In another embodiment, compounds of (2) or (3) above in which —B— is attached the pyrazole at 4- or 5-position thereof, and salts thereof.

(5) In another embodiment, compounds of the formula (V) in which A is thiophene, furan or pyrrole, and salts thereof.

(6) In another embodiment, compounds of (5) above in which —(CH₂)ₙ— is attached the 5-membered heterocyclic group at 2-position thereof, and salts thereof.

(7) In another embodiment, compounds of the formula (V) in which A is thiophene, and salts thereof.

(8) In another embodiment, compounds of (7) above in which —(CH₂)ₙ— is attached the thiophene at 2-position thereof, and salts thereof.

(9) In another embodiment, compounds of the formula (V) or one of (1) to (8) above in which n is 0, and salts thereof.

(10) In another embodiment, compounds of the formula (V) or one of (1) to (9) above in which each of X and Y is CH, and salts thereof.

(11) In another embodiment, compounds of the formula (V) or one of (1) to (10) above in which R² is combined with R³ to represent ═O, and salts thereof.

(12) In another embodiment, compounds of the formula (V) or one of (1) to (11) above in which B represents an alkylene chain having 2 to 4 carbon atoms which optionally has a substituent selected from the group consisting of an alkyl group having 1 to 8 carbon atoms and an alkyl group having 1 to 8 carbon atoms and a halogen atom substituent, and salts thereof.

(13) In another embodiment, compounds of the formula (V) or one of (1) to (12) above in which B is an ethylene chain, and salts thereof.

(14) In another embodiment, compounds of the formula (V) or one of (1) to (13) above in which R¹ and R⁴ are the same or different and each represents a hydrogen atom, an alkyl group having 1 to 8 carbon atoms, an alkoxy group having 1 to 8 carbon atoms, a halogen atom, an alkyl group having 1 to 8 carbon atoms and a halogen atom substituent, or an alkoxy group having 1 to 8 carbon atoms and a halogen atom substituent, and salts thereof.

(15) In another embodiment, compounds of the formula (V) or one of (1) to (14) above in which R⁵ and R⁶ are the same or different and each represents a hydrogen atom or an alkyl group having 1 to 8 carbon atoms, and salts thereof.

(16) In another embodiment, compounds of the formula (V) or one of (1) to (15) above in which the substituent optionally attached to the heterocyclic group for A is an alkyl group having 1 to 8 carbon atoms or an alkyl group having 1 to 8 carbon atoms and a halogen atom substituent, and salts thereof.

(17) In another embodiment, compounds of the formula (VI) in which X¹ is CH, and salts thereof.

(18) In another embodiment, compounds of the formula (II) in which R₁₁-phenyl or R¹¹-pyridyl is attached to the pyrazole at 1-position, and salts thereof.

(19) In another embodiment, compounds of the formula (VI) in which R¹¹-phenyl or R¹¹-pyridyl is attached to the pyrazole at 3-position, and salts thereof.

(20) In another embodiment, compounds of the formula (VI) or one of (17) to (19) above in which —(CH₂)_{q}C(═W¹)— is attached to the pyrazole at 4- or 5-position, and salts thereof.

(21) In another embodiment, compounds of the formula (VI) or one of (17) to (20) above in which W¹ is an oxygen atom, and salts thereof.

(22) In another embodiment, compounds of the formula (VI) or one of (17) to (21) above in which R¹¹ and R¹³ are the same or different and each represents a hydrogen atom, an alkyl group having 1 to 8 carbon atoms, an alkoxy group having 1 to 8 carbon atoms, a halogen atom, an alkyl group having 1 to 8 carbon atoms and a halogen atom substituent, or an alkoxy group having 1 to 8 carbon atoms and a halogen atom substituent, and salts thereof.

(23) In another embodiment, compounds of the formula (VI) or one of (17) to (21) above in which R¹¹ and R¹³ are the same or different and each represents an alkyl group having 1 to 8 carbon atoms or an alkyl group having 1 to 8 carbon atoms and a halogen atom substituent, and salts thereof.

(24) In another embodiment, compounds of the formula (VI) or one of (17) to (23) above in which R¹⁴ and R¹⁵ are the same or different and each represents a hydrogen atom or an alkyl group having 1 to 8 carbon atoms, and salts thereof.

(25) In another embodiment, compounds of the formula (VI) or one of (17) to (24) above in which R¹² represents a hydrogen atom, an alkyl group having 1 to 8 carbon atoms, an alkoxy group having 1 to 8 carbon atoms, an alkyl group having 1 to 8 carbon atoms and a halogen atom substituent, or an alkoxy group having 1 to 8 carbon atoms and a halogen atom substituent, and salts thereof.

(26) In another embodiment, compounds of the formula (VI) or one of (17) to (24) above in which R¹² represents an alkyl group having 1 to 8 carbon atoms, or an alkyl group having 1 to 8 carbon atoms and a halogen atom substituent, and salts thereof.

(27) In another embodiment, compounds of the formula (VI) or one of (17) to (26) above in which q is 2, and salts thereof.

(28) In another embodiment, compounds of the formula (VII) in which X² is CH, and salts thereof.

(29) In another embodiment, compounds of the formula (VII) in which R²¹-phenyl or R²¹-pyridyl is attached to the thiophene at 2-position, and salts thereof.

(30) In another embodiment, compounds of the formula (VII) or (28) or (29) above in which W² is an oxygen atom, and salts thereof.

(31) In another embodiment, compounds of the formula (VII) or one of (28) to (30) above in which R²¹ and R²³ are the same or different and each represents a hydrogen atom, an alkyl group having 1 to 8 carbon atoms, an alkoxy group having 1 to 8 carbon atoms, a halogen atom, an alkyl group having 1 to 8 carbon atoms and a halogen atom substituent, or an alkoxy group having 1 to 8 carbon atoms and a halogen atom substituent, and salts thereof.

(32) In another embodiment, compounds of the formula (VII) or one of (28) to (30) above in which R²¹ and R²³ are the same or different and each represents an alkyl group having 1 to 8 carbon atoms or an alkyl group having 1 to 8 carbon atoms and a halogen atom substituent, and salts thereof.

(33) In another embodiment, compounds of the formula (VII) or one of (28) to (32) above in which R²⁴ and R²⁵ are the same or different and each represents a hydrogen atom or an alkyl group having 1 to 8 carbon atoms, and salts thereof.

(34) In another embodiment, compounds of the formula (VII) or one of (28) to (33) above in which R²² represents a hydrogen atom, an alkyl group having 1 to 8 carbon atoms, an alkoxy group having 1 to 8 carbon atoms, an alkyl group having 1 to 8 carbon atoms and a halogen atom substituent, or an alkoxy group having 1 to 8 carbon atoms and a halogen atom substituent, and salts thereof.

(35) In another embodiment, compounds of the formula (VII) or one of (28) to (33) above in which R²² represents an alkyl group having 1 to 8 carbon atoms, or an alkyl group having 1 to 8 carbon atoms and a halogen atom substituent, and salts thereof.

(36) In another embodiment, compounds of the formula (VII) or one of (28) to (33) above in which r is 2, and salts thereof.

The compounds of the formulas (V), (VI) and (VII) can be pharmacologically acceptable salts such as alkali metal salts, for example, sodium salts, potassium salts, or lithium salts.

The compounds of the formulas (V), (VI) and (VII) can be present in the optically active forms, and in the form of optical isomers such as compounds of a racemic form or geometric isomers such as compounds of a cis- or trans form.

The non-limiting examples of the compounds of the formulae (V), (VI) and (VII) are:

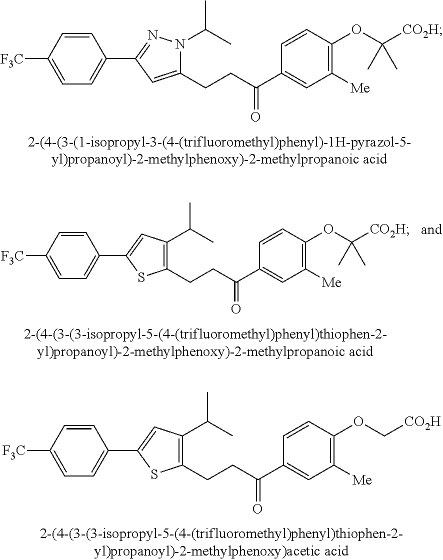

Compounds of the Formulae (VIII) and (IX)

In the formula (VIII), examples of the alkyl groups having 1 to 8 carbon atoms for R¹, R², R³, R⁴, R⁵, R⁶, R⁷, R⁸, R⁹, R¹⁰, R¹¹, R¹², R¹³ and R¹⁴ include methyl, ethyl, propyl, isopropyl, butyl, i-butyl, t-butyl, pentyl and hexyl.

Examples of the alkenyl groups having 2 to 8 carbon atoms for R¹, R², R³, R⁶ and R⁷ include vinyl and allyl.

Examples of the alkynyl groups having 2 to 8 carbon atoms for R¹, R², R³, R⁶ and R⁷ include propargyl.

Examples of the alkoxy groups having 1 to 8 carbon atoms for R¹, R², and R³ include methoxy, ethoxy, propoxy, isopropoxy, butoxy, i-butoxy, t-butoxy, pentyloxy and hexyloxy.

Examples of the halogen atoms for R¹, R², and R³ include fluorine, chlorine, and bromine.

Examples of the alkyl groups having 1 to 8 carbon atoms which are substituted with a halogen atom for R¹, R², R³, R⁴, R⁵, R⁶, and R⁷ include methyl, ethyl, propyl, isopropyl, butyl, and t-butyl which are substituted with 1 to 3 halogen atoms such as fluorine, chlorine, and bromine. In one embodiment, substituents are trifluoromethyl, chloromethyl, 2-chloroethyl, 2-bromoethyl, and 2-fluoroethyl.

Examples of the alkoxy groups having 1 to 8 carbon atoms which are substituted with a halogen atom for R¹, R², and R³ include methoxy, ethoxy, propoxy, isopropoxy, butoxy, and t-butoxy which are substituted with 1 to 3 halogen atoms such as fluorine, chlorine, and bromine. In one embodiment, substituents are trifluoromethyloxy, chloromethyloxy, 2-chloroethyloxy, 2-bromoethyloxy, and 2-fluoroethyloxy.

Examples of the acyl groups having 2 to 8 carbon atoms for R¹, R² and R³ include acetyl and propionyl.

Examples of the aryl groups having 6 to 10 carbon atoms for R¹, R² and R³ include phenyl.

Examples of the 5- or 6-membered heterocyclic groups for R¹, R² and R³ include pyridyl.

In the formula (IX), the alkyl groups having 1 to 8 carbon atoms, alkenyl groups having 2 to 8 carbon atoms, alkynyl groups having 2 to 8 carbon atoms, alkoxy groups having 1 to 8 carbon atoms, halogen atoms, alkyl groups having 1 to 8 carbon atoms which are substituted with a halogen atom, alkoxy groups having 1 to 8 carbon atoms which are substituted with a halogen atom, hydroxyls, nitros, acyl groups having 2 to 8 carbon atoms, aryl groups having 6 to 10 carbon atoms, and 5- or 6-membered hetero-cyclic groups for R²¹, R²² and R²³ can be those described for R¹, R² and R³ in the formula (VIII).

In the formula (IX), the alkyl groups having 1 to 8 carbon atoms and alkyl groups having 1 to 8 carbon atoms which are substituted with a halogen atom for R²⁴ and R²⁵ can be those described for R⁴ and R⁵ in the formula (VIII).

It should be noted that R¹, R² and R³ in the formula (VIII) and R²¹, R²² and R²³ in the formula (IX) can be attached to the benzene ring or the like in numbers of 1 to 3 in which the same or different groups can be attached to the same ring.

(1) In one embodiment, the compounds of the formula (VIII) and their salts are in which A is CH.

(2) In another embodiment, the compounds of the formula (VIII), the compounds of (1) above, and their salts are in which B is an oxygen atom.

(3) In another embodiment, the compounds of the formula (VIII), the compounds of (1) or (2) above, and their salts are in which W¹ is a bond.

(4) In another embodiment, the compounds of the formula (VIII), the compounds of (1) or (2) above, and their salts are in which W¹ is methylene or C(═O).

(5) In another embodiment, the compounds of the formula (VIII), the compounds of (1) to (4) above, and their salts are in which X and Y are different from each other and each is an oxygen atom, a sulfur atom or a nitrogen atom.

(6) In another embodiment, the compounds of the formula (VIII), the compounds of (1) to (4) above, and their salts are in which X is a sulfur atom and Y is a nitrogen atom.

(7) In another embodiment, the compounds of the formula (VIII), the compounds of (1) to (6) above, and their salts are in which Z¹ is an oxygen atom or a sulfur atom.

(8) In another embodiment, the compounds of the formula (VIII), the compounds of (1) to (7) above, and their salts are in which R¹, R² and R³ independently is a hydrogen atom, an alkyl group having 1 to 8 carbon atoms, an alkenyl group having 2 to 8 carbon atoms, an alkoxy group having 1 to 8 carbon atoms, a halogen atom, an alkyl group having 1 to 8 carbon atoms which is substituted with a halogen atom, or an alkoxy group having 1 to 8 carbon atoms which is substituted with a halogen atom.

(9) In another embodiment, the compounds of the formula (VIII), the compounds of (1) to (8) above, and their salts are in which each of R⁴ and R⁵ independently is a hydrogen atom or methyl.

(10) In another embodiment, the compounds of the formula (VIII), the compounds of (1) to (9) above, and their salts are in which each of R⁶ and R⁷ independently is a hydrogen atom or an alkyl group having 1 to 8 carbon atoms.

(11) In another embodiment, the compounds of the formula (VIII), the compounds of (1) to (10) above, and their salts are in which n is an integer of 2 to 4.

(12) In another embodiment, the compounds of the formula (VIII), the compounds of (1) to (10) above, and their salts are in which n is 2.

(13) In another embodiment, the compounds of the formula (IX) and their salts are in which W² is a bond.

(14) In another embodiment, the compounds of the formula (IX), the compounds of (13) above, and their salts are in which R²¹, R²² and R²³ independently is a hydrogen atom, an alkyl group having 1 to 8 carbon atoms, an alkenyl group having 2 to 8 carbon atoms, an alkoxy group having 1 to 8 carbon atoms, a halogen atom, an alkyl group having 1 to 8 carbon atoms which is substituted with a halogen atom, or an alkoxy group having 1 to 8 carbon atoms which is substituted with a halogen atom.

(15) In another embodiment, the compounds of the formula (IX), the compounds of (13) or (14) above, and their salts are in which each of R²⁴ and R²⁵ independently is a hydrogen atom or methyl.

The compounds provided herein which are represented by the formula (VIII) or (IX) can be in the form of a pharmacologically acceptable salts such as alkali metal salts. e.g., salts of sodium, potassium and lithium.

The compounds provided herein can be in the optically active forms, and in the form of optical isomers such as compounds of a racemic form or geometric isomers such as compounds of a cis- or trans form.

The non-limiting examples of the compounds of the formula (VIII) are:

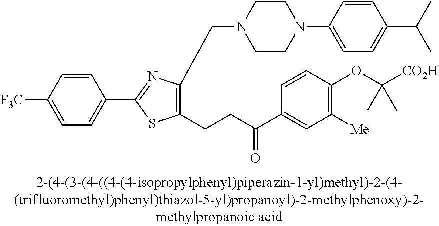

Compounds of the Formulae (X) and (XI)

In the formula (X), the alkyl group having 1 to 8 carbon atoms for R¹, R², R³, R⁴, R⁵, a substituent possibly attached to the 5-membered hetero ring of A, and a substituent possibly attached to the alkylene chain having 1 to 8 carbon atoms can be methyl, ethyl, propyl, isopropyl, butyl, i-butyl, t-butyl, pentyl, or hexyl.

In one embodiment, the alkenyl group having 2 to 8 carbon atoms for R¹, R² and a substituent is attached to the 5-membered hetero ring of A and is vinyl or allyl.

In one embodiment, the alkynyl group having 2 to 8 carbon atoms for R¹, R², wherein a substituent is attached to the 5-membered hetero ring of A and is propargyl.

In one embodiment, the 3- to 7-membered cycloalkyl group for R¹, R² wherein a substituent is attached to the 5-membered hetero ring of A. In another embodiment, a substituent is attached to the alkylene chain having 1 to 8 carbon atoms and is cyclopentyl or cyclohexyl.

In one embodiment, the alkoxy group having 1 to 8 carbon atoms for R¹, R², wherein a substituent is attached to the 5-membered hetero ring of A. In another embodiment, a substituent is attached to the alkylene chain having 1 to 8 carbon atoms and is methoxy, ethoxy, propoxy, isopropoxy, butoxy, i-butoxy, t-butoxy, pentyloxy, or hexyloxy.

In one embodiment. R¹ and R² is halogen, a substituent is attached to the 5-membered hetero ring of A. In another embodiment, a substituent is attached to the alkylene chain having 1 to 8 carbon atoms and is fluorine, chlorine, or bromine.

In one embodiment, the alkyl group having 1 to 8 carbon atoms and a halogen substituent for R¹, R², R⁵, wherein a substituent is attached to the 5-membered hetero ring of A and is methyl, ethyl, propyl, isopropyl, butyl or t-butyl which has 1 to 3 halogen substituents such as fluorine, chlorine or bromine. In another embodiment, the substituents are trifluoromethyl, chloromethyl, 2-chloroethyl, 2-bromoethyl, and 2-fluoroethyl.

In one embodiment, the alkoxy group having 1 to 8 carbon atoms and a halogen substituent for R¹, R², wherein a substituent is attached to the 5-membered hetero ring of A and is methoxy, ethoxy, propoxy, isopropoxy, butyloxy or t-butyloxy which has 1 to 3 halogen substituents such as fluorine, chlorine or bromine. In one embodiment, the substituents are trifluoromethyloxy, chloromethyloxy, 2-chloroethyloxy, 2-bromoethyloxy, and 2-fluoroethyloxy.

In one embodiment, the aryl group having 6 to 10 carbon atoms for R¹, R², and a substituent is attached to the 5-membered hetero ring of A and is phenyl.

In one embodiment, the 5- or 6-membered heterocyclic group for R¹, R², and a substituent is attached to the 5-membered hetero ring of A and is pyridyl.

In one embodiment, the alkyl group having 1 to 8 carbon atoms and 3- to 7-membered cycloalkyl group for R¹, R², and a substituent is attached to the 5-membered hetero ring of A and is methyl, ethyl, propyl, isopropyl, butyl, i-butyl, t-butyl, pentyl, or hexyl which has a cyclopropyl substituent, a cyclopentyl substituent, or a cyclohexyl substituent.

In one embodiment, the aralkyl having an aryl moiety of 6 to 10 carbon atoms and an alkylene moiety of 1 to 8 carbon atoms for R¹, R², and a substituent is attached to the 5-membered hetero ring of A and is benzyl or phenethyl.

In one embodiment, the alkyl group having 1 to 8 carbon atoms and 5- or 6-membered heterocyclic group for R¹, R², and a substituent is attached to the 5-membered hetero ring of A and is methyl, ethyl, propyl, isopropyl, butyl, i-butyl, t-butyl, pentyl, or hexyl which has a pyridyl substituent.

In one embodiment, the 5-membered hetero ring, which may have a substituent for A, is pyrazole or thiophene having a substituent. In another embodiment, pyrazole is having a substituent.

In one embodiment, the alkylene chain having 1 to 8 carbon atoms which has substituent for B is an alkylene chain having 1 to 4 carbon atoms. In another embodiment, the alkylene chain is an ethylene chain or a propylene chain.

In one embodiment, n is 0.

In one embodiment, in the formula (XI), the halogen atom, alkyl group having 1 to 8 carbon atoms, alkoxy group having 1 to 8 carbon atoms, alkyl group having 1 to 8 carbon atoms and a halogen substituent, and alkoxy group having 1 to 8 carbon atoms and a halogen substituent for R¹¹ and R¹² can be those described hereinbefore for R¹ and R² of the formula (X).

In one embodiment, the alkyl group having 1 to 8 carbon atoms for R¹³ and R¹⁴ can be those described hereinbefore for R³ and R⁴ of the formula (X).

In one embodiment, the halogen atom, alkyl group having 1 to 8 carbon atoms, alkoxy group having 1 to 8 carbon atoms, alkyl group having 1 to 8 carbon atoms and a halogen substituent, and alkoxy group having 1 to 8 carbon atoms and a halogen substituent which is attached to pyrazole or thiophene for A¹ in the formula (XI) are those described hereinbefore for the substituents attached to the 5-membered hetero ring of A of the formula (X).

In one embodiment, R¹ of the formula (X) and R¹¹ of the formula (XI), the benzene ring or the like can have 1 to 3 number of R¹ or R¹¹ which are the same or differ-ent from each other. In another embodiment, the benzene ring or the like can have 1 to 3 substituents other than a hydrogen atom.

In one embodiment, R² of the formula (X) and R¹² of the formula (XI), the benzene ring of the benzisoxazole ring or the like can have 1 to 3 number of R² or R¹² which are the same or different from each other. In another embodiment, the benzene ring of the benzisoxazole ring or the like can have 1 to 3 substituents other than a hydrogen atom.

In one embodiment, the substituent attached to the 5-membered hetero ring for A of the formula (X) and the substituent attached to pyrazole or thiophene for A¹ of the formula (XI) can be present in 1 or 2 number which can be the same or different from each other.

(1) In one embodiment, the compound or a salt thereof represented by the formula (X), in which each of W¹ and W² represents CH.

(2) In another embodiment, the compound or a salt thereof represented by the formula (X), in which W¹ represents CH and W² represents a nitrogen atom.

(3) In another embodiment, the compound or a salt thereof represented by the formula (X) or according to (1) or (2) above, in which X represents a nitrogen atom.

(4) In another embodiment, the compound or a salt thereof represented by the formula (X) or according to (1) or (2) above, in which X represents a nitrogen atom and Y represents an oxygen atom.

(5) In another embodiment, the compound or a salt thereof represented by the formula (I) or according to (1) or (2) above, in which X represents CH and Y represents an oxygen atom.

(6) In another embodiment, the compound or a salt thereof represented by the formula (X) or according to any one of (1) to (5) above, in which Z represents an oxygen atom or a sulfur atom.

(7) In another embodiment, the compound or a salt thereof represented by the formula (X) or according to any one of (1) to (6) above, in which each of R¹ and R² independently represents a hydrogen atom, a halogen atom, a hydroxyl group, a nitro group, an amino group, an alkyl group having 1 to 8 carbon atoms, an alkoxy group having 1 to 8 carbon atoms, an alkyl group having 1 to 8 carbon atoms and a halogen substituent, or an alkoxy group having 1 to 8 carbon atoms and a halogen substituent.

(8) In another embodiment, the compound or a salt thereof represented by the formula (X) or according to any one of (1) to (7) above, in which each of R³ and R⁴ independently represents a hydrogen atom or an alkyl group having 1 to 8 carbon atoms.

(9) In another embodiment, the compound or a salt thereof represented by the formula (X) or according to any one of (1) to (8) above, in which A represents pyrazole, thiophene or furan which may have a substituent selected from the group consisting of a halogen atom, a hydroxyl group, a nitro group, an amino group, an alkyl group having 1 to 8 carbon atoms, a 3- to 7-membered cycloalkyl group, an alkenyl group having 2 to 8 carbon atoms, an alkynyl group having 2 to 8 carbon atoms, an alkoxy group having 1 to 8 carbon atoms, an alkyl group having 1 to 8 carbon atoms and a 3- to 7-membered cycloalkyl substituent, an alkyl group having 1 to 8 carbon atoms and a halogen substituent, an alkoxy group having 1 to 8 carbon atoms and a halogen substituent, an aryl group having 6 to 10 carbon atoms, a 5- or 6-membered heterocyclic group, an aralkyl group having an aryl moiety of 6 to 10 carbon atoms and an alkylene moiety of 1 to 8 carbon atoms, and an alkyl group having 1 to 8 carbon atoms and a 5- or 6-membered heterocyclic substituent.

(10) In another embodiment, the compound or a salt thereof represented by the formula (X) or according to any one of (1) to (8) above, in which A represents pyrazole, thiophene or furan which may have a substituent selected from the group consisting of a halogen atom, a hydroxyl group, a nitro group, an amino group, an alkyl group having 1 to 8 carbon atoms, an alkoxy group having 1 to 8 carbon atoms, an alkyl group having 1 to 8 carbon atoms and a halogen substituent, or an alkoxy group having 1 to 8 carbon atoms and a halogen substituent.

(11) In another embodiment, the compound or a salt thereof represented by the formula (X) or according to any one of (1) to (8) above, in which A represents pyrazole which may have a substituent selected from the group consisting of a halogen atom, a hydroxyl group, a nitro group, an amino group, an alkyl group having 1 to 8 car-bon atoms, an alkoxy group having 1 to 8 carbon atoms, an alkyl group having 1 to 8 carbon atoms and a halogen substituent, and an alkoxy group having 1 to 8 carbon atoms and a halogen substituent.

(12) In another embodiment, the compound or a salt thereof represented by the formula (X) or according to any one of (1) to (11) above, in which B represents an alkylene chain having 2 to 4 carbon atoms.

(13) In another embodiment, the compound or a salt thereof represented by the formula (X) or according to any one of (1) to (12) above, in which n is 0.

(14) In another embodiment, the compound or a salt thereof represented by the formula (XI), in which W³ represents CH.

(15) In another embodiment, the compound or a salt thereof represented by the formula (XI) or according to (14) above, in which A¹ represents pyrazole which may have a substituent selected from the group consisting of a halogen atom, a hydroxyl group, a nitro group, an amino group, an alkyl group having 1 to 8 carbon atoms, an alkoxy group having 1 to 8 carbon atoms, an alkyl group having 1 to 8 carbon atoms and a halogen substituent, or an alkoxy group having 1 to 8 carbon atoms and a halogen substituent.

(16) In another embodiment, the compound or a salt thereof represented by the formula (XI) or according to (14) or (15) above, in which m is 2 or 3.

The compound of the formula (X) or (XI) can be in the form of a pharmacologically acceptable salt such as a salt of an alkali metal such as sodium, potassium, or lithium.

The compounds provided are in the optically active forms, and in the form of optical isomers such as compounds of a racemic form or geometric isomers such as compounds of a cis- or trans form.

The non-limiting examples of the compounds of the formulae (X) and (XI) are:

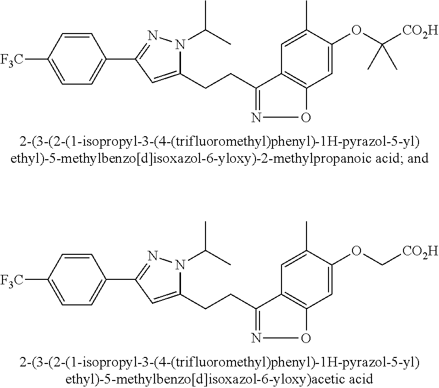

Compounds of the Formulae (XII), (XIII) and (XIV)

In one embodiment, in the formula (XII), R¹, R², R³, R⁴, R⁵, R⁶, R⁷, R⁸, R⁹, R¹⁰, R¹¹, R¹², R¹³, a substituent of the five-membered heterocyclic ring represented by A, and a substituent of the C₁₋₈ alkylene, C₂₋₈ alkenylene or C₂₋₈ alkynylene chain represented by B can be C₁₋₈ alkyl. Examples of the C₁₋₈ alkyl include methyl, ethyl, propyl, isopropyl, butyl, isobutyl, t-butyl, pentyl and hexyl.

In one embodiment, R¹, R², R³, and a substituent of the five-membered heterocyclic ring represented by A can be C₂₋₈; alkenyl. Examples of the C₂₋₈ alkenyl include vinyl and allyl.

In one embodiment, R¹, R², and a substituent of the five-membered heterocyclic ring represented by A can be C₂₋₈ alkynyl. Examples of the C₂₋₈ alkynyl include propargyl.

In one embodiment, R¹, R², a substituent of the five-membered heterocyclic ring represented by A, and a substituent of the C₁₋₈ alkylene, C₂₋₈ alkenylene or C₂₋₈ alkynylene chain represented by B can be C₁₋₈ alkoxy. Examples of the C₁₋₈ alkoxy include methoxy, ethoxy, propoxy, isopropoxy, butoxy, isobutoxy, t-butoxy, pentyloxy, and hexyloxy.

In one embodiment, R¹, R², a substituent of the five-membered heterocyclic ring represented by A, and a substituent of the C₁₋₈ alkylene, C₂₋₈ alkenylene or C₂₋₈ alkynylene chain represented by B can be halogen. Examples of the halogen include fluorine, chlorine, and bromine.

In one embodiment, R¹, R², R⁵, and a substituent of the five-membered heterocyclic ring represented by A can be C₁₋₈ alkyl substituted with halogen. Examples of the C₁₋₈ alkyl substituted with halogen include methyl, ethyl, propyl, isopropyl, butyl, and t-butyl which are substituted with 1-3 halogens such as fluorine, chlorine, and bromine. Preferred are trifluoromethyl, chloromethyl, 2-chloroethyl, 2-bromoethyl, and 2-fluoroethyl.

In one embodiment, R¹, R², and a substituent of the five-membered heterocyclic ring represented by A can be C₁₋₈ alkoxy substituted with halogen. Examples of the C₁₋₈ alkoxy substituted with halogen include methoxy, ethoxy, propoxy, isopropoxy, butoxy, and t-butoxy which are substituted with 1-3 halogen atoms such as fluorine atom, chlorine atom, or bromine atom. In one embodiment, R¹, R², and a substituent of the five-membered heterocyclic ring are trifluoromethoxy, chloromethoxy, 2-chloroethoxy, 2-bromoethoxy, and 2-fluoroethoxy.

In one embodiment, R¹, R², R⁵, and a substituent of the five-membered heterocyclic ring represented by A can be C₂₋₈ acyl. Examples of the C₂₋₈ acyl include acetyl and propionyl.

In one embodiment, R¹, R², and a substituent of the five-membered heterocyclic ring represented by A can be C₆₋₁₀ aryl. Examples of the C₆₋₁₀ aryl include phenyl.

In one embodiment, R¹, R², and a substituent of the five-membered heterocyclic ring represented by A can be a five-membered or six-membered heterocyclic group. Examples of the five-membered or six-membered heterocyclic group include pyridyl.

In one embodiment, R⁵ can be C₁₋₈ alkyl substituted with C₁₋₈ alkoxy. Examples of the C₁₋₈ alkyl substituted with C₁₋₈ alkoxy include methyl, ethyl, propyl, isopropyl, butyl, isobutyl, t-butyl, pentyl and hexyl which are substituted with methoxy, ethoxy, propoxy, isopropoxy, butoxy, isobutoxy, t-butoxy, pentyloxy, or hexyloxy.

In one embodiment, R⁵ can be cycloalkyl of three-membered to seven-membered ring. Examples of the cycloalkyl of three-membered to seven-membered ring include cyclopropyl, cyclobutyl, cyclopentyl, and cyclohexyl.

In one embodiment, R⁵ can be C₁₋₈ alkyl substituted with cycloalkyl of three-membered to seven-membered ring. Examples of the C₁₋₈ alkyl substituted with cycloalkyl of three-membered to seven-membered ring include methyl, ethyl, propyl, isopropyl, butyl, isobutyl, t-butyl, pentyl and hexyl which are substituted with cyclopropyl, cyclobutyl, cyclopentyl, or cyclohexyl.

In one embodiment, R⁵ can be C₁₋₈ alkyl substituted with phenyl. Examples of the C₁₋₈ alkyl substituted with phenyl include benzyl and phenethyl.

In one embodiment, a substituent of the C₁₋₈ alkylene, C₂₋₈ alkenylene or C₂₋₈ alkynylene chain represented by B can be cycloalkyl of three-membered to seven-membered ring. Examples of the cycloalkyl of three-membered to seven-membered ring include cyclopropyl, cyclobutyl, cyclopentyl, and cyclohexyl.

In one embodiment, in the formula (XIII), R^{1a}, R^{2a}, and a substituent of five-membered heterocyclic ring represented by A^{a} can be C₁₋₈ alkyl, C₁₋₈ alkoxy, halogen, C₁₋₈ alkyl substituted with halogen, C₁₋₈ alkoxy substituted with halogen, and C₂₋₈ acyl. Examples of them are the same as the examples of R¹, R², and the substituent of the five-membered heterocyclic ring represented by A in the formula (XII).

In one embodiment, in the formula (XIV), R^{1b}, R^{2b}, and a substituent of five-membered heterocyclic ring represented by A^{b} can be C₁₋₈ alkyl, C₁₋₈ alkoxy, halogen, C₁₋₈ alkyl substituted with halogen, C₁₋₈ alkoxy substituted with halogen, and C₇₋₈ acyl. Examples of them are the same as the examples of R¹, R², and the substituent of the five-membered heterocyclic ring represented by A in the formula (XII).

In one embodiment, in the formula (XIV), R^{3b} can be C₁₋₈ alkyl. Examples are the same as the examples of R⁵ in the formula (XII).

In one embodiment, each of R¹, R² in the formula (XII), R^{1a}, R^{2a} in the formula (XIII), R^{1b} and R^{2b} in the formula (XIV) can be one to three groups attached to the rings, such as benzene ring. The two or three groups can be different from each other.

(1) In one embodiment, provided is a compound having the formula (XII) or a salt thereof, wherein each of W¹ and W² is CH.

(2) In one embodiment, provided is a compound having the formula (XII), a salt thereof, a compound defined in (1), or a salt thereof, wherein X is CR⁶R⁷.

(3) In one embodiment, provided is a compound having the formula (XII), a salt thereof, a compound defined in (1), or a salt thereof, wherein X is CH₂.

(4) In one embodiment, provided is a compound having the formula (XII), a salt thereof, a compound defined in (1), or a salt thereof, wherein X is NR⁵.

(5) In one embodiment, provided is a compound having the formula (XII), a salt thereof, a compound defined in (1), or a salt thereof, wherein X is NH.

(6) In one embodiment, provided is a compound having the formula (XII), a salt thereof, a compound defined in (1), or a salt thereof, wherein X is NR⁵, and R⁵ is C₁₋₈ alkyl.

(7) In one embodiment, provided is a compound having the formula (XII), a salt thereof, a compound defined in one of (1) to (6), or a salt thereof, wherein Y is CH₂.

(8) In one embodiment, provided is a compound having the formula (XII), a salt thereof, a compound defined in one of (1) to (7), or a salt thereof, wherein Z is carboxyl.

(9) In one embodiment, provided is a compound having the formula (XII), a salt thereof, a compound defined in one of (1) to (8), or a salt thereof, wherein G is O.

(10) In one embodiment, provided is a compound having the formula (XII), a salt thereof, a compound defined in one of (1) to (9), or a salt thereof, wherein A is thiazole, which can be substituted with a substituent selected from the group consisting of C₁₋₈ alkyl, C₂₋₈ alkenyl, C₂₋₈ alkynyl, C₁₋₈ alkoxy, halogen, C₁₋₈ alkyl substituted with halogen, C₁₋₈ alkoxy substituted with halogen, hydroxyl, nitro, C₂₋₈ acyl, C₆₋₁₀ aryl, and a five-membered or six-membered heterocyclic group.

(11) In one embodiment, provided is a compound having the formula (XII), a salt thereof, a compound defined in one of (1) to (10), or a salt thereof, wherein B is ethylene chain.

(12) In one embodiment, provided is a compound having the formula (XII), a salt thereof, a compound defined in one of (1) to (11), or a salt thereof, wherein each of R¹ and R² independently is hydrogen, C₁₋₈ alkyl, C₂₋₈ alkenyl, C₁₋₈ alkoxy, halogen, C₁₋₈ alkyl substituted with halogen, or C₁₋₈ alkoxy substituted with halogen.

(13) In one embodiment, provided is a compound having the formula (XII), a salt thereof, a compound defined in one of (1) to (11), or a salt thereof, wherein each of R¹ and R² independently is hydrogen, C₁₋₈ alkyl, halogen, or C₁₋₈ alkyl substituted with halogen.

(14) In one embodiment, provided is a compound having the formula (XII), a salt thereof, a compound defined in one of (1) to (13), or a salt thereof, wherein each of R³ and R⁴ is hydrogen.

(15) In one embodiment, provided is a compound having the formula (XII), a salt thereof, a compound defined in one of (1) to (14), or a salt thereof, wherein m is 0.

(16) In one embodiment, provided is a compound having the formula (XIII) or a salt thereof, wherein G^{a} is 0.

(17) In one embodiment, provided is a compound having the formula (XIII), a salt thereof, a compound defined in (16), or a salt thereof, wherein A^{a} is thiazole, which can be substituted with a substituent selected from the group consisting of C₁₋₈ alkyl, C₁₋₈ alkoxy, halogen, C₁₋₈ alkyl substituted with halogen, C₁₋₈ alkoxy substituted with halogen, hydroxyl, nitro, and C₂₋₈ acyl.

(18) In one embodiment, provided is a compound having the formula (XIII), a salt thereof, a compound defined in (16) or (17), or a salt thereof, wherein B^{a} is ethylene chain.

(19) In one embodiment, provided is a compound having the formula (XIII), a salt thereof, a compound defined in one of (16) to (18), or a salt thereof, wherein each of R^{1a} and R^{2a} independently is hydrogen, C₁₋₈ alkyl, C₁₋₈ alkoxy, halogen, C₁₋₈ alkyl substituted with halogen, or C₁₋₈ alkoxy substituted with halogen.

(20) In one embodiment, provided is a compound having the formula (XIV) or a salt thereof, wherein G^{b} is 0.

(21) In one embodiment, provided is a compound having the formula (XIV), a salt thereof a compound defined in (20), or a salt thereof, wherein A^{b} is thiazole, which can be substituted with a substituent selected from the group consisting of C₁₋₈ alkyl, C₁₋₈ alkoxy, halogen, C₁₋₈ alkyl substituted with halogen, C₁₋₈ alkoxy substituted with halogen, hydroxyl, nitro, and C₂₋₈ acyl.

(22) In one embodiment, provided is a compound having the formula (XIV), a salt thereof, a compound defined in (20) or (21), or a salt thereof, wherein B^{b} is ethylene chain.

(23) In one embodiment, provided is a compound having the formula (XIV), a salt thereof, a compound defined in one of (20) to (22), or a salt thereof, wherein each of R^{1b} and R^{2b} independently is hydrogen, C₁₋₈ alkyl, C₁₋₈ alkoxy, halogen, C₁₋₈ alkyl substituted with halogen, or C₁₋₈ alkoxy substituted with halogen.

The compounds having the formulae (XII), (XIII), or (XIV) can be present in the form of a pharmaceutically acceptable salt. Examples of the salt include an alkali metal salt, such as sodium salt, potassium salt and lithium salt.

The compounds having the formulae (XII), (XIII), or (XIV) can also be present in the form of an optical isomer such as enantiomer or racemic body, or a geometrical isomer such as cis or trans. Also provided are isomers of these compounds.

The non-limiting examples of the compounds of the formulae (XII), (XIII), and (XIV) are:

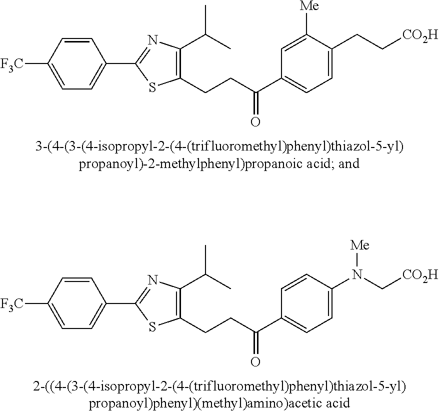

Compounds of the Formulae (XV) and (XVI)

In one embodiment, the compounds having the formula (XV) or a salt thereof, wherein both W¹ and W² are CH.

In another embodiment, the compounds having the formula (XV) or a salt thereof, wherein X is CR⁴R⁵, CH₂, or NR³, wherein R³ is an alkyl group having 1 to 8 carbon atoms. In another embodiment, R³ is a methyl group;

In another embodiment, the compounds having the formula (XV) or a salt thereof, wherein Y is CH₂.

In another embodiment, the compounds having the formula (XV) or a salt thereof, wherein Z is a carboxylic group.

In another embodiment, the compounds having the formula (XV) or a salt thereof, wherein A is thiazole or oxazole which may have a substituent selected from the group consisting of an alkyl group having 1 to 8 carbon atoms, an alkenyl group having 2 to 8 carbon atoms, an alkynyl group having 2 to 8 carbon atoms, an alkyl group having 1 to 8 carbon atoms and a halogen atom substituent, an aryl group having 6 to 10 carbon atoms or a 5 or 6-membered heterocyclic group; pyrazole which may have a substituent selected from the group consisting of an alkyl group having 1 to 8 carbon atoms, an alkenyl group having 2 to 8 carbon atoms, an alkynyl group having 2 to 8 carbon atoms, an alkyl group having 1 to 8 carbon atoms and a halogen atom substituent, an aryl group having 6 to 10 carbon atoms or a 5 or 6-membered heterocyclic group.

In another embodiment, the compounds having the formula (XV) or a salt thereof, wherein B is an ethylene chain.

In another embodiment, the compounds having the formula (XV) or a salt thereof, wherein D is N.

In another embodiment, the compounds having the formula (XV) or a salt thereof, wherein E is O.

In another embodiment, the compounds having the formula (XV) or a salt thereof, wherein each of R¹ and R² is independently H, an alkyl group having 1 to 8 carbon atoms, an alkenyl group having 2 to 8 carbon atoms, an alkoxy group having 1 to 8 carbon atoms, a halogen atom, an alkyl group having 1 to 8 carbon atoms and a halogen atom substituent or an alkoxy group having 1 to 8 carbon atoms and a halogen atom substituent.

In another embodiment, the compounds having the formula (XV) or a salt thereof, wherein m is 0.

In one embodiment, the compounds having the formula (XVI) or a salt thereof, wherein R¹³ is an alkyl group having 1 to 8 carbon atoms. In another embodiment, R¹³ is a methyl group.

In one embodiment, the compounds having the formula (XVI) or a salt thereof, wherein p is 1.

In one embodiment, the compounds having the formula (XVI) or a salt thereof, wherein A¹ is thiazole, oxazole or phenyl which may have a substituent selected from the group consisting of an alkyl group having 1 to 8 carbon atoms or an alkyl group having 1 to 8 carbon atoms and a halogen atom substituent. In another embodiment, A¹ is thiazole which may have an alkyl group having 1 to 8 carbon atoms as a substituent.

In one embodiment, the compounds having the formula (XVI) or a salt thereof, wherein B¹ is an ethylene chain.

In one embodiment, the compounds having the formula (XVI) or a salt thereof, wherein R¹¹ is an alkyl group having 1 to 8 carbon atoms, a halogen atom or an alkyl group having 1 to 8 carbon atoms and a halogen atom substituent.

In one embodiment, the compounds having the formula (XVI) or a salt thereof, wherein R¹² is H, an alkyl group having 1 to 8 carbon atoms or an alkyl group having 1 to 8 carbon atoms and a halogen atom substituent.

The compounds having the formula (XVI) can also be present in the form of an optical isomer such as enantiomer or racemic body, or a geometrical isomer such as cis or trans. Also provided are isomers of these compounds.

The compounds having the formulae (XV) and (XVI) can also be present in the form of an optical isomer such as enantiomer or racemic body, or a geometrical isomer such as cis or trans. Also provided are isomers of these compounds.

The non-limiting examples of the compounds of the formulae (XV) and (XVI) are:

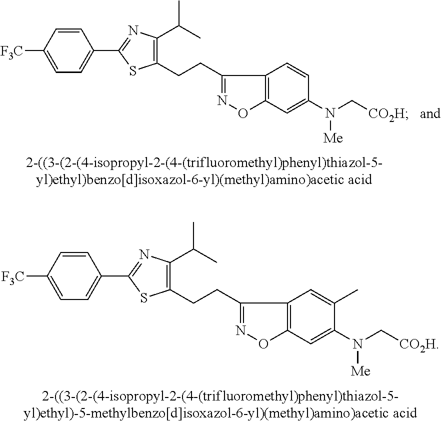

Compounds of the Formula (XVII)

In one embodiment, the compounds having the formula (XVII) or a salt thereof, wherein R²³ is an alkyl group having 1 to 8 carbon atoms or an alkyl group having 1 to 8 carbon atoms and a halogen atom substituent. In another embodiment, R²³ is a methyl group.

In one embodiment, the compounds having the formula (XVII) or a salt thereof, wherein q is an integer of 1 to 4. In another embodiment, q is 1.

In one embodiment, the compounds having the formula (XVII) or a salt thereof, wherein R²⁰ is an alkyl group having 1 to 8 carbon atoms. In another embodiment, R²⁰ is methyl.

In one embodiment, the compounds having the formula (XVII) or a salt thereof, wherein B² is an alkylene chain having 2 to 4 carbon atoms. In another embodiment, B² is an ethylene chain.

In one embodiment, the compounds having the formula (XVII) or a salt thereof, wherein each of R²¹ and R²² is independently H, an alkyl group having 1 to 8 carbon atoms, a halogen atom, an alkyl group having 1 to 8 carbon atoms and a halogen atom substituent. In another embodiment, R²¹ is an alkyl group having 1 to 8 carbon atoms, a halogen atom or an alkyl group having 1 to 8 carbon atoms and a halogen atom substituent. In yet another embodiment, R²² is H, an alkyl group having 1 to 8 carbon atoms or an alkyl group having 1 to 8 carbon atoms and a halogen atom substituent.

In one embodiment, the compounds having the formula (XVII), wherein N(R²³)((CH₂)_{q}—CO₂H) is attached to the 6th position of benzisoxazole.

The compounds having the formula (XVII) can also be present in the form of an optical isomer such as enantiomer or racemic body, or a geometrical isomer such as cis or trans. Also provided are isomers of these compounds.

Compounds of the Formula (XVIII)

In one embodiment, in the formula (XVIII), R¹ represents hydrogen, halogen, hydroxyl, nitro, amino, cyano, carboxyl, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, a 5- or 6-membered heterocyclic group, an aralkyl group having an aryl moiety of 6-10 carbon atoms and an alkylene moiety of 1-8 carbon atoms, or an alkyl group having 1-8 carbon atoms and a 5- or 6-membered heterocyclic substituent.

In one embodiment, in the formula (XVIII), R² represents hydrogen, an alkyl group having 1-8 carbon atoms, an alkenyl group having 2-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an acyl group having 2-8 carbon atoms, an aryl group having 6-10 carbon atoms, or an aralkyl group having an aryl moiety of 6-10 carbon atoms and an alkylene moiety of 1-8 carbon atoms.

In one embodiment, in the formula (XVIII), each of R³, R⁴, R⁵ and R⁶ independently represents hydrogen, an alkyl group having 1-8 carbon atoms, or an alkyl group having 1-8 carbon atoms and having a halogen substituent.

In one embodiment, in the formula (XVIII), X is oxygen, sulfur or NR⁷, R⁷ representing hydrogen, an alkyl group having 1-8 carbon atoms, an alkyl group having 1-8 carbon atoms and having a halogen substituent, an aralkyl group having an aryl moiety of 6-10 carbon atoms and an alkylene moiety of 1-8 carbon atoms, an acyl group having 2-8 carbon atoms, or an alkenyl group having 2-8 carbon atoms.

In one embodiment, in the formula (XVIII), Y is oxygen, sulfur, NR⁸ or a bond, R⁸ representing hydrogen, an alkyl group having 1-8 carbon atoms, an alkyl group having 1-8 carbon atoms and having a halogen substituent, an acyl group having 2-8 carbon atoms, or an alkenyl group having 2-8 carbon atoms.

In one embodiment, in the formula (XVIII), p is 0 or 1.

In one embodiment, in the formula (XVIII), A is oxygen CH₂, N—NH₂ or N—OR⁹, R⁹ representing hydrogen, an alkyl group having 1-8 carbon atoms, an alkyl group having 1-8 carbon atoms and having a halogen substituent, an acyl group having 2-8 carbon atoms, an alkenyl group having 2-8 carbon atoms, or an aralkyl group having an aryl moiety of 6-10 carbon atoms and an alkylene moiety of 1-8 carbon atoms.

In one embodiment, in the formula (XVIII), B represents, in the case of p=1, a benzene ring having or not having a substituent selected from the group consisting of halogen, hydroxyl, nitro, amino, an alkyl group having 1-8 carbon atoms, 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an alkoxy group having 1-8 carbon atoms and having a halogen substituent, an acyl group having 2-8 carbon atoms, an aryl group having 6-10 carbon atoms, or an aralkyl group having an aryl moiety of 6-10 carbon atoms and an alkylene moiety of 1-8 carbon atoms, and, in the case of p=0, a condensed ring selected from the group consisting of indole, benzofuran, benz-isoxazole and 1,2-benzisothiazole, in which said condensed ring has or does not have a substituent selected from the group consisting of halogen, hydroxyl, nitro, amino, an alkyl group having 1-8 carbon atoms, 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an alkoxy group having 1-8 carbon atoms and having a halogen substituent, an acyl group having 2-8 carbon atoms, an aryl group having 6-10 carbon atoms, or an aralkyl group having an aryl moiety of 6-10 carbon atoms and an alkylene moiety of 1-8 carbon atoms.

In one embodiment, in the formula (XVIII), Y is bonded to the benzene ring of B.

In one embodiment, in the formula (XVIII), —(C(R³)(R⁴))ₘ— is bonded to the condensed ring of B at its 3-position.

In one embodiment, in the formula (XVIII), m is an integer of 1 to 4.

In one embodiment, in the formula (XVIII), n is an integer of 0 to 5.

In one embodiment, in the formula (XVIII), Y is a bond in the case of n=0.

The compounds having the formulae (XVIII) can also be present in the form of an optical isomer such as enantiomer or racemic body, or a geometrical isomer such as cis or trans. Also provided are isomers of these compounds.

Compounds of the Formula (XIX)

In one embodiment, in the formula (XIX), R¹¹ represents hydrogen, halogen, hydroxyl, nitro, amino, cyano, carboxyl, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, a 5- or 6-membered heterocyclic group, an aralkyl group having an aryl moiety of 6-10 carbon atoms and an alkylene moiety of 1-8 carbon atoms, or an alkyl group having 1-8 carbon atoms and a 5- or 6-membered heterocyclic substituent.

In one embodiment, in the formula (XIX), R¹² represents hydrogen, an alkyl group having 1-8 carbon atoms, an alkenyl group having 2-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an acyl group having 2-8 carbon atoms, an aryl group having 6-10 carbon atoms, or an aralkyl group having an aryl moiety of 6-10 carbon atoms and an alkylene moiety of 1-8 carbon atoms.

In one embodiment, in the formula (XIX), each of R¹³, R¹⁴, R¹⁵ and R¹⁶ independently represents hydrogen, an alkyl group having 1-8 carbon atoms, or an alkyl group having 1-8 carbon atoms and having a halogen substituent.

In one embodiment, in the formula (XIX), Y¹ is oxygen, sulfur, NR¹⁸ or a bond, R¹⁸ representing hydrogen, an alkyl group having 1-8 carbon atoms, an alkyl group having 1-8 carbon atoms and having a halogen substituent, an acyl group having 2-8 carbon atoms, or an alkenyl group having 2-8 carbon atoms.

In one embodiment, in the formula (XIX), A¹ is oxygen CH₂, N—NH₂ or N—OR¹⁹, R¹⁹ representing hydrogen, an alkyl group having 1-8 carbon atoms, an alkyl group having 1-8 carbon atoms and having a halogen substituent, an acyl group having 2-8 carbon atoms, an alkenyl group having 2-8 carbon atoms, or an aralkyl group having an aryl moiety of 6-10 carbon atoms and an alkylene moiety of 1-8 carbon atoms.

In one embodiment, in the formula (XIX), Q¹ represents hydrogen, halogen, hydroxyl, nitro, amino, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, or an aralkyl group having an aryl moiety of 6-10 carbon atoms and an alkylene moiety of 1-8 carbon atoms.

In one embodiment, in the formula (XIX), r is an integer of 1 to 4.

In one embodiment, in the formula (XIX), s is an integer of 1 to 5.

The compounds having the formulae (XIX) can also be present in the form of an optical isomer such as enantiomer or racemic body, or a geometrical isomer such as cis or trans. Also provided are isomers of these compounds.

The non-limiting example of the compounds of the formula (XIX) is:

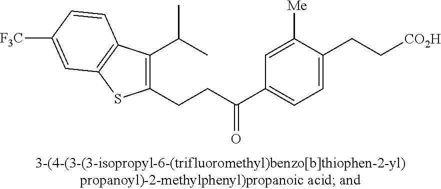

Compounds of the Formula (XX)

In one embodiment, in the formula (XX), R²¹ represents hydrogen, halogen, hydroxyl, nitro, amino, cyano, carboxyl, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, a 5- or 6-membered heterocyclic group, an aralkyl group having an aryl moiety of 6-10 carbon atoms and an alkylene moiety of 1-8 carbon atoms, or an alkyl group having 1-8 carbon atoms and a 5- or 6-membered heterocyclic substituent.

In one embodiment, in the formula (XX), R²² represents hydrogen, an alkyl group having 1-8 carbon atoms, an alkenyl group having 2-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an acyl group having 2-8 carbon atoms, an aryl group having 6-10 carbon atoms, or an aralkyl group having an aryl moiety of 6-10 carbon atoms and an alkylene moiety of 1-8 carbon atoms.

In one embodiment, in the formula (XX), each of R²³, R²⁴, R²⁵ and R²⁶ independently represents hydrogen, an alkyl group having 1-8 carbon atoms, or an alkyl group having 1-8 carbon atoms and having a halogen substituent.

In one embodiment, in the formula (XX), Y² is oxygen, sulfur, NR²⁸ or a bond, R²⁸ representing hydrogen, an alkyl group having 1-8 carbon atoms, an alkyl group having 1-8 carbon atoms and having a halogen substituent, an acyl group having 2-8 carbon atoms, or an alkenyl group having 2-8 carbon atoms.

In one embodiment, in the formula (XX), Q² represents hydrogen, halogen, hydroxyl, nitro, amino, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, or an aralkyl group having an aryl moiety of 6-10 carbon atoms and an alkylene moiety of 1-8 carbon atoms.

In one embodiment, in the formula (XX), t is an integer of 1 to 4.

In one embodiment, in the formula (XX), u is an integer of 1 to 5.

The compounds having the formulae (XX) can also be present in the form of an optical isomer such as enantiomer or racemic body, or a geometrical isomer such as cis or trans. Also provided are isomers of these compounds.

Synthesis of an exemplary compound represented by formula (XX) is presented below:

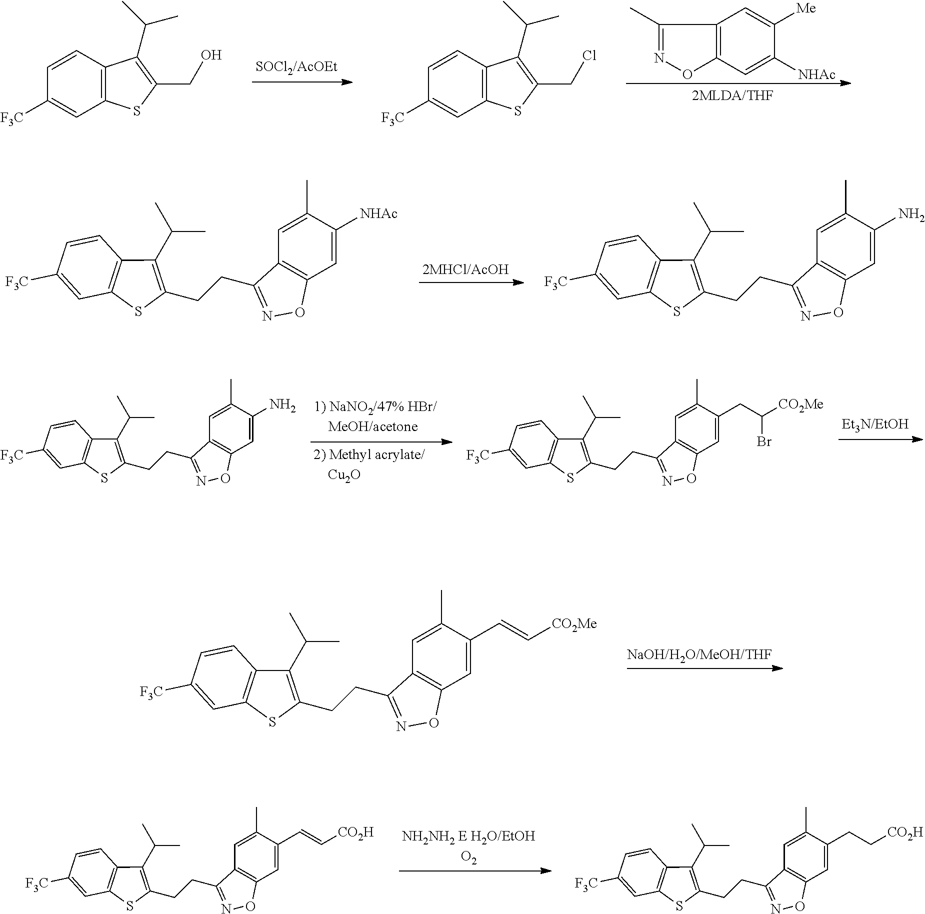

Synthesis of another exemplary compound represented by formula (XX) is presented below:

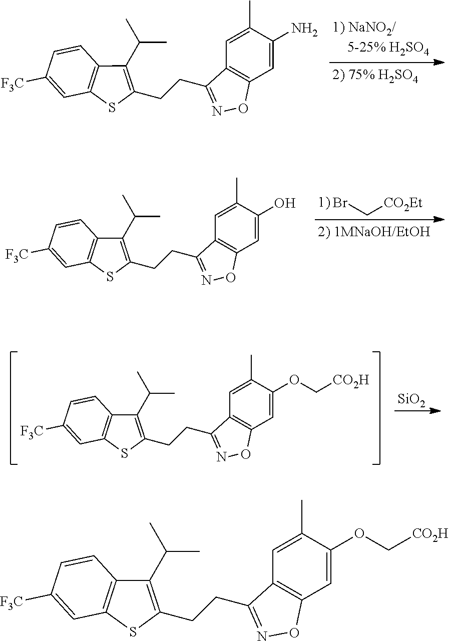

The non-limiting examples of the compounds of the formula (XX) are:

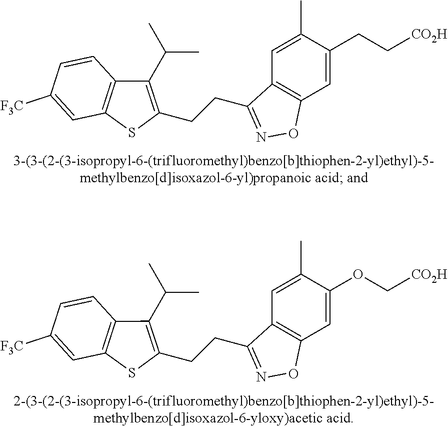

Methods of Use

In one embodiment, provided is a method of treatment, prevention, or amelioration of one or more symptoms of insulin resistance, involving administering a PPARδ agonist. Such methods reduce, alleviate or eliminate antihyperglycemic and insulin-sensitizing effects.

In another embodiment, provided is a method of treatment, prevention, or amelioration of one or more symptoms of disorders associated with increased oxidative muscle fibers, involving administering a PPAR-6 agonist.

In another embodiment, provided is a method of treatment, prevention, or amelioration of one or more symptoms of inflammation, involving administering a PPARδ agonist.

In another embodiment, provided is a method of treatment, prevention, or amelioration of one or more symptoms of diseases or disorders associated with functional constriction or actual obstruction of a kidney blood vessel, involving administering a PPARδ agonist. In these methods, the PPARδ agonist improves blood circulation in one or both kidneys.

In another embodiment, provided is a method of treatment, prevention, or amelioration of one or more symptoms of disorders associated with lung inflammation, involving administering a PPAR-δ agonist.

In another embodiment, provided is a method for treating diseases of the lung, including but not limited to, chronic obstructive airways disease (COAD), chronic obstructive pulmonary disease (COPD), adult onset asthma, emphysema or juvenile onset, and asthma, involving administering a PPARδ agonist.

In another embodiment, provided is a method for treating other inflammatory conditions where an inflammatory response is present such as inflammatory vascular diseases (including but not limited to atherosclerosis, coronary or peripheral vascular disease, myocardial infarction or stroke), inflammatory bowel disease (Crohn's disease and ulcerative colitis), systemic inflammatory disorders (Lupus Erythematosus) or inflammatory rheumatic disorders (including but not limited to rheumatoid arthritis or psoriatic joint disease), and inflammatory diseases of the lung, involving administering a PPARδ agonist.

In another embodiment, provided is a method for treating disorders or manifestations of insulin and glucose metabolism (including insulin resistance, diabetes, the metabolic syndrome, hypoglycemia, high blood pressure, obesity or dyslipidemia, protection of pancreatic beta cells and prevention of microvascular and macrovascular disorders), involving administering a PPARδ agonist.

In another embodiment, provided is a method for treating central or abdominal or visceral obesity, in which weight loss is required or desired, involving administering a PPARδ agonist.

In another embodiment, provided is a method for treating disorders of the kidney, including but not limited to, renal ischemia, involving administering a PPARδ agonist.

In another embodiment, provided is a method for treating mitochondrial disorders, including but not limited to, myoclonus twiching, epilepsy, ragged red fibers (RRF), hearing loss, exercise intolerance, dementia, and lactic acidosis, comprising administering a PPARδ agonist.

In another embodiment, provided is a method for treating hair loss comprising administering a PPARδ agonist.

In another embodiment, provided is a method for wound healing comprising administering a PPARδ agonist.

In another embodiment, in the methods provided, the use of a low dose of any selective PPARδ agonist with a selectivity of >500 over PPARα and PPARγ results that avoid the side effects associated with the use of PPARα and PPARγ agonists, or classical PPAR agonist side effects when used in conjuction to treatment of the disorders above.

Depending on the disease to be treated and the subject's condition, the compounds of Formulae I to XX, as well as any PPARδ agonist, including, but not limited to, GSK-501516 (Ligand/GSK), RWJ-800025 (JNJ/Metabolex), KD-3010 (Kalypsys, Inc.), BAY 68-5042 (Bayer), or compounds described in Bratton, L. D. et al., Bioorg. Med. Chem. Lett. 2007 (web edition) and Kasuga, J. I. et al., Bioorg. Med. Chem. 2007 (web edition) provided herein may be administered by oral or parenteral (e.g., intramuscular, intraperitoneal, intravenous, ICV, intracistemal injection or infusion, subcutaneous injection, or implant) routes of administration, and may be formulated, alone or together, in suitable dosage unit with pharmaceutically acceptable carriers, adjuvants and vehicles appropriate for each route of administration. In one embodiment, the compounds provided are administered orally.

In certain embodiments, in the methods provided herein, an appropriate dosage level of a PPARδ agonist for humans is about 0.1 mg/day to about 2500 mg/day and results in increase in pre-β-HDL levels while avoiding the side effects associated with the use of PPARα and PPARγ agonists, or classical PPAR agonist class side effects. In yet another embodiment, the dose is about 0.25 mg/day to about 500 mg/day. In yet another embodiment, the dose is about 0.5 mg/day to about 250 mg/day. In yet another embodiment, the dose is about 0.75 mg/day to about 50 mg/day. In yet another embodiment, the dose is about 1.0 mg/day to about 25 mg/day.

In another embodiment, in the methods provided, a dose of a PPARδ agonist for humans is about 0.001 mg/kg/day to about 25 mg/kg/day results in increase in pre-β-HDL levels while avoiding the side effects associated with the use of PPARα and PPARγ agonists, or classical PPAR agonist class side effects. In yet another embodiment, dose is about 0.005 mg/kg/day to about 15 mg/kg/day. In yet another embodiment, the dose is about 0.01 mg/kg/day to about 10 mg/kg/day. In yet another embodiment, the dose is about 0.5 mg/kg/day to about 5 mg/kg/day. In yet another embodiment, the dose is about 1.0 mg/kg/day to about 2.5 mg/kg/day, which may be administered in a single or divided doses. Within this range the dosage may be about 0.1 mg, about 0.25 mg, about 0.5 mg, about 0.75 mg, about 1.0 mg, about 2.5 mg, about 5 mg, about 7.5 mg, about 10 mg, about 15 mg, about 25 mg, about 50 mg, about 75 mg, about 100 mg, about 125 mg, about 150 mg, about 175 mg, about 200 mg, about 225 mg, or about 250 mg per day.

In another embodiment, in the methods provided, the low doses of the PPARδ agonists, such as 0.05 to 30 mg/kg/day in monkeys and 0.5 mg/day to 300 mg/day in humans do not cause significant side effects usually reported to be associated with the PPARδ agonists.

In another embodiment, compounds provided may be used in combination with any other active agents or pharmaceutical compositions where such combined therapy is useful to reduce plaque build-up and therefore treat the conditions related thereto.

Pharmaceutical Compositions

Provided herein are pharmaceutical compositions comprising one or more compounds of Formulae I to XX, as well as any PPARδ agonist, including, but not limited to, GW-501516 (Ligand/GSK), RWJ-800025 (JNJ/Metabolex), KD-3010 (Kalypsys, Inc.), BAY 68-5042 (Bayer), or compounds described in Bratton, L. D. et al., Bioorg. Med. Chem. Lett. 2007 (web edition) and Kasuga, J. I. et al., Bioorg. Med. Chem. 2007 (web edition) as active ingredients or a pharmaceutically acceptable salt, solvate, or prodrug thereof, in a pharmaceutically acceptable vehicle, carrier, diluent, or excipient, or a mixture thereof.

Provided herein are pharmaceutical compositions in modified release dosage forms, which comprise one or more compounds of Formulae I to XX, as well as any PPARδ agonist, including, but not limited to, GW-501516 (Ligand/GSK), RWJ-800025 (JNJ/Metabolex), KD-3010 (Kalypsys, Inc.), BAY 68-5042 (Bayer), or compounds described in Bratton, L. D. et al., Bioorg. Med. Chem. Lett. 2007 (web edition) and Kasuga, J. I. et al., Bioorg. Med. Chem. 2007 (web edition) or a pharmaceutically acceptable salt, solvate, or prodrug thereof; and one or more release controlling excipients as described herein. Suitable modified release dosage vehicles include, but are not limited to, hydrophilic or hydrophobic matrix devices, water-soluble separating layer coatings, enteric coatings, osmotic devices, multiparticulate devices, and combinations thereof. The pharmaceutical compositions may also comprise non-release controlling excipients.

Further provided herein are pharmaceutical compositions in enteric coated dosage fauns, which comprise one or more compounds of Formulae I to XX or a pharmaceutically acceptable salt, solvate, or prodrug thereof; and one or more release controlling excipients for use in an enteric coated dosage form. The pharmaceutical compositions may also comprise non-release controlling excipients.

Additionally provided are pharmaceutical compositions in a dosage form that has an instant releasing component and at least one delayed releasing component, and is capable of giving a discontinuous release of the compound in the form of at least two consecutive pulses separated in time from 0.1 up to 24 hours.

In one embodiment, the pharmaceutical compositions comprise one or more compounds of Formulae I to XX, as well as any PPARδ agonist including, but not limited to, GW-501516 (Ligand/GSK), RWJ-800025 (JNJ/Metabolex), KD-3010 (Kalypsys, Inc.), BAY 68-5042 (Bayer), or compounds described in Bratton, L. D. et al., Bioorg. Med. Chem. Lett. 2007 (web edition) and Kasuga, J. I. et al., Bioorg. Med. Chem. 2007 (web edition) or a pharmaceutically acceptable salt, solvate, or prodrug thereof; and one or more release controlling and non-release controlling excipients, such as those excipients suitable for a disruptable semi-permeable membrane and as swellable substances.

Provided herein also are pharmaceutical compositions in a dosage form for oral administration to a subject, which comprise one or more compounds of Formulae I to XX or a pharmaceutically acceptable salt, solvate, or prodrug thereof; and one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

Provided herein are pharmaceutical compositions that comprise about 0.1 mg/day to about 2500 mg/day of a PPARδ agonist. In yet another embodiment, pharmaceutical compositions comprise about 0.25 mg/day to about 500 mg/day of a PPARδ agonist. In yet another embodiment, pharmaceutical compositions comprise about 0.5 mg/day to about 250 mg/day of a PPARδ agonist. In yet another embodiment, pharmaceutical compositions comprise about 0.75 mg/day to about 50 mg/day of a PPARδ agonist. In yet another embodiment, pharmaceutical compositions comprise about 1.0 mg/day to about 25 mg/day of a PPARδ agonist.

In another embodiment, pharmaceutical compositions provided herein comprise about 0.001 mg/kg/day to about 25 mg/kg/day of a PPARδ agonist. In yet another embodiment, pharmaceutical compositions provided herein comprise about 0.005 mg/kg/day to about 15 mg/kg/day of a PPARδ agonist. In yet another embodiment, pharmaceutical compositions provided herein comprise about 0.01 mg/kg/day to about 10 mg/kg/day of a PPARδ agonist. In yet another embodiment, pharmaceutical compositions provided herein comprise about 0.5 mg/kg/day to about 5 mg/kg/day of a PPARδ agonist. In yet another embodiment, pharmaceutical compositions provided herein comprise about 1.0 mg/kg/day to about 2.5 mg/kg/day of a PPARδ agonist, which may be administered in a single or divided doses. The pharmaceutical compositions further comprise about 0.1% to about 2% sodium chloride, about 0.1% to about 2% ammonium acetate, about 0.001% to about 0.1% edetate disodium, about 0.1% to about 2% benzyl alcohol, with a pH of about 6 to about 8.

The pharmaceutical compositions provided herein may be provided in unit-dosage forms or multiple-dosage forms. Unit-dosage forms, as used herein, refer to physically discrete units suitable for administration to human and animal subjects and packaged individually as is known in the art. Each unit-dose contains a predetermined quantity of the active ingredient(s) sufficient to produce the desired therapeutic effect, in association with the required pharmaceutical carriers or excipients. Examples of unit-dosage forms include ampouls, syringes, and individually packaged tablets and capsules. Unit-dosage forms may be administered in fractions or multiples thereof. A multiple-dosage form is a plurality of identical unit-dosage forms packaged in a single container to be administered in segregated unit-dosage form. Examples of multiple-dosage forms include vials, bottles of tablets or capsules, or bottles of pints or gallons.

The compounds of Formulae I to XX, as well as any PPARδ agonist, including, but not limited to, GW-501516 (Ligand/GSK), RWJ-800025 (JNJ/Metabolex), KD-3010 (Kalypsys, Inc.), BAY 68-5042 (Bayer), or compounds described in Bratton, L. D. et al., Bioorg. Med. Chem. Lett. 2007 (web edition) and Kasuga, J. I. et al., Bioorg. Med. Chem. 2007 (web edition) provided herein may be administered alone, or in combination with one or more other compounds provided herein, or one or more other active ingredients. The pharmaceutical compositions that comprise compounds provided herein may be formulated in various dosage forms for oral administration. The pharmaceutical compositions may also be formulated as a modified release dosage form, including delayed-, extended-, prolonged-, sustained-, pulsatile-, controlled-, accelerated- and fast-, targeted-, programmed-release, and gastric retention dosage forms. These dosage forms can be prepared according to conventional methods and techniques known to those skilled in the art (see. Remington: The Science and Practice of Pharmacy, supra; Modified-Release Drug Deliver Technology, Rathbone et al., Eds., Drugs and the Pharmaceutical Science, Marcel Dekker, Inc.: New York, N.Y., 2002; Vol. 126).

The pharmaceutical compositions provided herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

Oral Administration

The pharmaceutical compositions provided herein may be provided in solid, semisolid, or liquid dosage forms for oral administration. As used herein, oral administration also include buccal, lingual, and sublingual administration. Suitable oral dosage forms include, but are not limited to, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, and syrups. In addition to the active ingredient(s), the pharmaceutical compositions may contain one or more pharmaceutically acceptable carriers or excipients, including, but not limited to, binders, fillers, diluents, disintegrants, wetting agents, lubricants, glidants, coloring agents, dye-migration inhibitors, sweetening agents, and flavoring agents.

Binders or granulators impart cohesiveness to a tablet to ensure the tablet remaining intact after compression. Suitable binders or granulators include, but are not limited to, starches, such as corn starch, potato starch, and pre-gelatinized starch (e.g., STARCH 1500); gelatin; sugars, such as sucrose, glucose, dextrose, molasses, and lactose; natural and synthetic gums, such as acacia, alginic acid, alginates, extract of Irish moss, Panwar gum, ghatti gum, mucilage of isabgol husks, carboxymethylcellulose, methylcellulose, polyvinylpyrrolidone (PVP), Veegum, larch arabogalactan, powdered tragacanth, and guar gum; celluloses, such as ethyl cellulose, cellulose acetate, carboxymethyl cellulose calcium, sodium carboxymethyl cellulose, methyl cellulose, hydroxyethylcellulose (HEC), hydroxypropylcellulose (HPC), hydroxypropyl methyl cellulose (HPMC); microcrystalline celluloses, such as AVICEL-PH-101, AVICEL-PH-103, AVICEL RC-581, AVICEL-PH-105 (FMC Corp., Marcus Hook, Pa.); and mixtures thereof. Suitable fillers include, but are not limited to, talc, calcium carbonate, microcrystalline cellulose, powdered cellulose, dextrates, kaolin, mannitol, silicic acid, sorbitol, starch, pre-gelatinized starch, and mixtures thereof. The binder or filler may be present from about 50 to about 99% by weight in the pharmaceutical compositions provided herein.

Suitable diluents include, but are not limited to, dicalcium phosphate, calcium sulfate, lactose, sorbitol, sucrose, inositol, cellulose, kaolin, mannitol, sodium chloride, dry starch, and powdered sugar. Certain diluents, such as mannitol, lactose, sorbitol, sucrose, and inositol, when present in sufficient quantity, can impart properties to some compressed tablets that permit disintegration in the mouth by chewing. Such compressed tablets can be used as chewable tablets.

Suitable disintegrants include, but are not limited to, agar; bentonite; celluloses, such as methylcellulose and carboxymethylcellulose; wood products; natural sponge; cation-exchange resins; alginic acid; gums, such as guar gum and Veegum HV; citrus pulp; cross-linked celluloses, such as croscarmellose; cross-linked polymers, such as crospovidone; cross-linked starches; calcium carbonate; microcrystalline cellulose, such as sodium starch glycolate; polacrilin potassium; starches, such as corn starch, potato starch, tapioca starch, and pre-gelatinized starch; clays; aligns; and mixtures thereof. The amount of disintegrant in the pharmaceutical compositions provided herein varies upon the type of formulation, and is readily discernible to those of ordinary skill in the art. The pharmaceutical compositions provided herein may contain from about 0.5 to about 15% or from about 1 to about 5% by weight of a disintegrant.

Suitable lubricants include, but are not limited to, calcium stearate; magnesium stearate; mineral oil; light mineral oil; glycerin; sorbitol; mannitol; glycols, such as glycerol behenate and polyethylene glycol (PEG); stearic acid; sodium lauryl sulfate; talc; hydrogenated vegetable oil, including peanut oil, cottonseed oil, sunflower oil, sesame oil, olive oil, corn oil, and soybean oil; zinc stearate; ethyl oleate; ethyl laureate; agar; starch; lycopodium; silica or silica gels, such as AEROSIL® 200 (W.R. Grace Co., Baltimore, Md.) and CAB-O-SIL® (Cabot Co. of Boston, Mass.); and mixtures thereof. The pharmaceutical compositions provided herein may contain about 0.1 to about 5% by weight of a lubricant.

Suitable glidants include colloidal silicon dioxide, CAB-O-SIL® (Cabot Co. of Boston, Mass.), and asbestos-free talc. Coloring agents include any of the approved, certified, water soluble FD&C dyes, and water insoluble FD&C dyes suspended on alumina hydrate, and color lakes and mixtures thereof. A color lake is the combination by adsorption of a water-soluble dye to a hydrous oxide of a heavy metal, resulting in an insoluble form of the dye. Flavoring agents include natural flavors extracted from plants, such as fruits, and synthetic blends of compounds which produce a pleasant taste sensation, such as peppermint and methyl salicylate. Sweetening agents include sucrose, lactose, mannitol, syrups, glycerin, and artificial sweeteners, such as saccharin and aspartame. Suitable emulsifying agents include gelatin, acacia, tragacanth, bentonite, and surfactants, such as polyoxyethylene sorbitan monooleate (TWEEN® 20), polyoxyethylene sorbitan monooleate 80 (TWEEN® 80), and triethanolamine oleate. Suspending and dispersing agents include sodium carboxymethylcellulose, pectin, tragacanth, Veegum, acacia, sodium carbomethylcellulose, hydroxypropyl methylcellulose, and polyvinylpyrolidone. Preservatives include glycerin, methyl and propylparaben, benzoic add, sodium benzoate and alcohol. Wetting agents include propylene glycol monostearate, sorbitan monooleate, diethylene glycol monolaurate, and polyoxyethylene lauryl ether. Solvents include glycerin, sorbitol, ethyl alcohol, and syrup. Examples of non-aqueous liquids utilized in emulsions include mineral oil and cottonseed oil. Organic acids include citric and tartaric acid. Sources of carbon dioxide include sodium bicarbonate and sodium carbonate.

It should be understood that many carriers and excipients may serve several functions, even within the same formulation.

The pharmaceutical compositions provided herein may be provided as compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which may be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. Film coating imparts the same general characteristics as sugar coating. Multiple compressed tablets are compressed tablets made by more than one compression cycle, including layered tablets, and press-coated or dry-coated tablets.

The tablet dosage forms may be prepared from the active ingredient in powdered, crystalline, or granular forms, alone or in combination with one or more carriers or excipients described herein, including binders, disintegrants, controlled-release polymers, lubricants, diluents, and/or colorants. Flavoring and sweetening agents are especially useful in the formation of chewable tablets and lozenges.

The pharmaceutical compositions provided herein may be provided as soft or hard capsules, which can be made from gelatin, methylcellulose, starch, or calcium alginate. The hard gelatin capsule, also known as the dry-filled capsule (DFC), consists of two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. The soft gelatin shells may contain a preservative to prevent the growth of microorganisms. Suitable preservatives are those as described herein, including methyl- and propyl-parabens, and sorbic acid. The liquid, semisolid, and solid dosage forms provided herein may be encapsulated in a capsule. Suitable liquid and semisolid dosage forms include solutions and suspensions in propylene carbonate, vegetable oils, or triglycerides. Capsules containing such solutions can be prepared as described in U.S. Pat. Nos. 4,328,245; 4,409,239; and 4,410,545. The capsules may also be coated as known by those of skill in the art in order to modify or sustain dissolution of the active ingredient.

The pharmaceutical compositions provided herein may be provided in liquid and semisolid dosage forms, including emulsions, solutions, suspensions, elixirs, and syrups. An emulsion is a two-phase system, in which one liquid is dispersed in the form of small globules throughout another liquid, which can be oil-in-water or water-in-oil. Emulsions may include a pharmaceutically acceptable non-aqueous liquids or solvent, emulsifying agent, and preservative. Suspensions may include a pharmaceutically acceptable suspending agent and preservative. Aqueous alcoholic solutions may include a pharmaceutically acceptable acetal, such as a di(lower alkyl)acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs are clear, sweetened, and hydroalcoholic solutions. Syrups are concentrated aqueous solutions of a sugar, for example, sucrose, and may also contain a preservative. For a liquid dosage form, for example, a solution in a polyethylene glycol may be diluted with a sufficient quantity of a pharmaceutically acceptable liquid carrier, e.g., water, to be measured conveniently for administration.

Other useful liquid and semisolid dosage forms include, but are not limited to, those containing the active ingredient(s) provided herein, and a dialkylated mono- or poly-alkylene glycol, including, 1,2-dimethoxymethane, diglyme, triglyme, tetraglyme, polyethylene glycol-350-dimethyl ether, polyethylene glycol-550-dimethyl ether, polyethylene glycol-750-dimethyl ether, wherein 350, 550, and 750 refer to the approximate average molecular weight of the polyethylene glycol. These formulations may further comprise one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, bisulfite, sodium metabisulfite, thiodipropionic acid and its esters, and dithiocarbamates.

The pharmaceutical compositions provided herein for oral administration may be also provided in the forms of liposomes, micelles, microspheres, or nanosystems. Miccellar dosage forms can be prepared as described in U.S. Pat. No. 6,350,458.

The pharmaceutical compositions provided herein may be provided as non-effervescent or effervescent, granules and powders, to be reconstituted into a liquid dosage form. Pharmaceutically acceptable carriers and excipients used in the non-effervescent granules or powders may include diluents, sweeteners, and wetting agents. Pharmaceutically acceptable carriers and excipients used in the effervescent granules or powders may include organic acids and a source of carbon dioxide.

Coloring and flavoring agents can be used in all of the above dosage forms.

The pharmaceutical compositions provided herein may be formulated as immediate or modified release dosage forms, including delayed-, sustained, pulsed-, controlled, targeted-, and programmed-release forms.

The pharmaceutical compositions provided herein may be co-formulated with other active ingredients which do not impair the desired therapeutic action, or with substances that supplement the desired action, such as antacids, proton pump inhibitors, and H₂-receptor antagonists.

Controlled-Release Dosage Forms

The pharmaceutical compositions in an osmotic controlled-release dosage form may further comprise additional conventional excipients as described herein to promote performance or processing of the formulation.

The osmotic controlled-release dosage forms can be prepared according to conventional methods and techniques known to those skilled in the art (see, Remington: The Science and Practice of Pharmacy, supra; Santus and Baker, J. Controlled Release 1995, 35, 1-21; Verma et al., Drug Development and Industrial Pharmacy 2000, 26, 695-708; Verma et al., J. Controlled Release 2002, 79, 7-27).

In certain embodiments, the pharmaceutical compositions provided herein are formulated as AMT controlled-release dosage form, which comprises an asymmetric osmotic membrane that coats a core comprising the active ingredient(s) and other pharmaceutically acceptable excipients. See, U.S. Pat. No. 5,612,059 and WO 2002/17918. The AMT controlled-release dosage forms can be prepared according to conventional methods and techniques known to those skilled in the art, including direct compression, dry granulation, wet granulation, and a dip-coating method.

In certain embodiment, the pharmaceutical compositions provided herein are formulated as ESC controlled-release dosage form, which comprises an osmotic membrane that coats a core comprising the active ingredient(s), hydroxylethyl cellulose, and other pharmaceutically acceptable excipients.

Dosing

In certain embodiments, compounds provided are administered once daily in a single or divided dose in the amount of about 0.001 to about 25 mg/kg, where kg refers to a subject's body weight.

In certain embodiments, compounds provided are administered once daily in a single or divided dose in the amount of about 0.005 to about 15 mg/kg.

In certain embodiments, compounds provided are administered once daily in a single or divided dose in the amount of about 0.01 to about 10 mg/kg.

In certain embodiments, compounds provided are administered once daily in a single or divided dose in the amount of about 0.5 to about 5 mg/kg.

In certain embodiments, compounds provided are administered once daily in a single or divided dose in the amount of about 1.0 to about 2.5 mg/kg.

In certain embodiments, compounds provided are administered once daily in a single or divided dose in the amount of about 0.1 mg, about 0.25 mg, about 0.5 mg, about 0.75 mg, about 1.0 mg, about 2.5 mg, about 5.0 mg, about 7.5 mg, about 10 mg, about 15 mg, or about 25 mg.

Additional Compounds

In one embodiment, any compound possessing PPARδ agonist activity may be used. In another embodiment, compounds that are selective PPARδ agonists are used. Exemplary compounds include, but are not limited to, GW-501516 (Ligand/GSK), RW1-800025 (JNJ/Metabolex), KD-3010 (Kalypsys, Inc.) and BAY 68-5042 (Bayer).

Incorporated herein by reference in their entireties are the U.S. Pat. Nos. 6,787,552, 7,078,422, 7,265,137, 7,119,104, and 7,402,597 assigned to Nippon Chemiphar Co. Ltd., disclosing PPARδ agonists.

Provided below are the following non-limiting examples.

### EXAMPLES

### Example 1

### 1A. 4-[3-[4-Isopropyl-2-[4-(trifluoromethyl)phenyl]-5-thiazolyl]propionyl]-2-methylphenoxyacetic acid

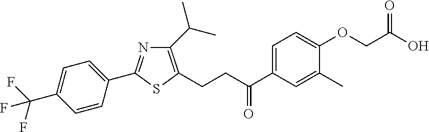

### (1) 1-(4-Hydroxy-3-methylphenyl)-3-[4-isopropyl-2-[4-(trifluoromethyl)phenyl]-5-thiazolyl]propan-1-one

4-Isopropyl-2-[4-(trifluoromethyl)phenyl]thiazole-5-methanol (70 g) was dissolved in EtOAc (0.7 L), and to the mixture SOCl₂ (32.3 g) was added dropwise. After 1 h of stirring at room temperature, the mixture was diluted with water and extracted with EtOAc. The organic layer was washed with aqueous NaHCO₃ and brine, and then dried over Na₂SO₄. The solvent was evaporated and the residue was dried in vacuo to give 5-chloromethyl-4-isopropyl-2-[4-(trifluoromethyl)phenyl]thiazole (73.5 g, yield 98.9%) as a white solid. Under nitrogen atmosphere, NaNH₂ (9.74 g) was suspended in THF (270 mL), and to the mixture was added the solution of ethyl 3-(4-benzyloxy-3-methylphenyl)-3-oxopropionate (0.65 g) in THF (270 mL). After stirring at room temperature for 0.5 h, the solution of 5-chloromethyl-4-isopropyl-2-[4-(trifluoromethyl)phenyl]thiazole (73.2 g) in THF (270 mL) was added dropwise and the mixture was refluxed for 14 h. The solvent was evaporated, and to the residue was added AcOH (270 mL) and CHCl₃ (134 mL) and the mixture was refluxed for 8 h. After cooling to room temperature, the resultant suspension was stirred in ice bath for 3 h. The filtrated crystal was dried in vacuo to give 1-(4-hydroxy-3-methylphenyl)-3-[4-isopropyl-2-[4-(trifluoromethyl)phenyl]-5-thiazolyl]propan-1-one (80 g, yield 88.7% from ethyl 3-(4-benzyloxy-3-methylphenyl)-3-oxopropionate) as a pale yellow solid.

¹H-NMR (CDCl₃, 400 MHz): 1.33 (d, 6H, J=7 Hz), 2.29 (s, 3H), 3.14 (dq, 1H, J=7 Hz, J=7 Hz), 3.2-3.3 (m, 4H), 5.35 (s, 1H), 6.80 (d, 1H, J=8 Hz), 7.63 (d, 2H, J=8 Hz), 7.74 (dd, 1H, J=2, 8 Hz), 7.79 (d, 1H, J=2 Hz), 7.89 (d, 2H, J=8 Hz).

### (2) Ethyl 4-[3-[4-isopropyl-2-[4-(trifluoromethyl)phenyl]-5-thiazolyl]propionyl]-2-methylphenoxyacetate

1-(4-Hydroxy-3-methylphenyl)-3-[4-isopropyl-2-[4-(trifluoromethyl)phenyl]-5-thiazolyl]propan-1-one (79.5 g) and Cs₂CO₃ (65.7 g) were suspended in acetone (0.8 L), and to the mixture was added ethyl bromoacetate (31.7 g). After refluxing for 2 h, the solvent was evaporated and to the residue was added water to extract with EtOAc. The organic layer was washed with brine and dried over Na₂SO₄. The solvent was evaporated to give crude product, which was recrystallized from n-hexane to give ethyl 4-[3-[4-isopropyl-2-[4-(trifluoromethyl)phenyl]-5-thiaz-olyl]propionyl]-2-methylphenoxyacetate (81.2 g, yield 85.2%) as a white crystal.

¹H-NMR (CDCl₃, 400 MHz) δ: 1.30 (t, 3H, J=7 Hz), 1.33 (d, 6H, J=7 Hz), 2.33 (s, 3H), 3.15 (dq, 1H, J=7 Hz, J=7 Hz), 3.2-3.3 (m, 4H), 4.27 (q, 2H, J=7 Hz), 4.71 (s, 2H), 6.71 (d, 1H, J=8 Hz), 7.64 (d, 2H, J=8 Hz), 7.75 (dd, 1H, J=2, 8 Hz), 7.81 (d, 1H, J=2 Hz), 8.00 (d, 2H, J=8 Hz).

### (3) [4-[3-[2-(4-Trifluoromethyl)phenyl-4-iso-propyl-5-thiazolyl]propionyl]--2-methylphenoxy]acetic acid

Ethyl 4-[3-[4-isopropyl-2-[4-(trifluoromethyl)phenyl]-5-thiazolyl]propionyl]-2-methylphenoxy-acetate (80 g) was suspended in EtOH (400 mL), and to the mixture was added the solution of NaOH (12.33 g) in water (400 mL). After stirring at room temperature for 2 h, to the mixture was added HCl (2 mol/L) to extract with EtOAc. The organic layer was washed with water and brine, then dried over Na₂SO₄. The solvent was evaporated to give the crude product, which was recrystallized from THF/n-hexane to give 4-[3-[4-isopropyl]-2-[4-((trifluoromethyl)phenyl]-5-thiazolyl]propionyl]-2-methylphenoxy-acetic acid (63 g, yield 83.1%) as a white crystal.

White powder (mp: 145-155° C.)

¹H-NMR (CDCl₃, 400 MHz) δ: 1.33 (d, 6H, J=7 Hz), 2.32 (s, 3H), 3.15 (dq, 1H, J=7 Hz, J=7 Hz), 3.2-3.3 (m, 4H), 4.76 (s, 2H), 6.75 (d, 1H, J=8 Hz), 7.64 (d, 2H, J=8 Hz), 7.81 (dd, 1H, J=2, 8 Hz), 7.82 (d, 1H, J=2 Hz), 8.00 (d, 2H, J=8 Hz).

### 1B. [3-[2-[3-Isopropyl-6-(trifluoromethyl)benzothiophen-2-yl]ethyl]-5-methyl-benzisoxazol-6-yloxy]acetic acid

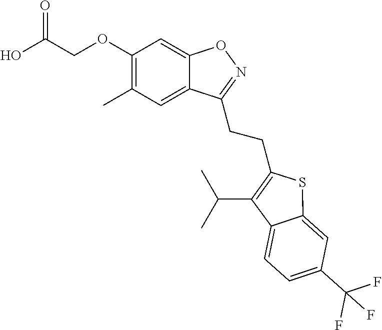

### (1) 6-Acetamido-[3-[2-[3-isopropyl-6-(trifluoromethyl)benzothiophen-2-yl]ethyl]-5-methyl]benzisoxazole

6-Acetamido-3,5-dimethylbenzisoxazole (381 mg, 1.87 mmol) was dissolved in dry THF (15 mL). To the solution, 2M LDA (2.3 mL, 4.6 mmol) was added dropwise for 20 min at −78° C. under N₂, and the mixture was stirred for 15 min at −78° C., to which THF solution (5.0 mL) of 2-(chloromethyl)-3-isopropyl-6(trifluoromethyl)benzothiophene (655 mg, 2.24 mmol) was added dropwise for 20 min. The mixture was stirred for 1 h under the same conditions, and warmed to room temperature. A saturated NH₄Cl aq and ethyl acetate were added to reaction mixture. The organic layer was washed with water, brine, dried over Na₂SO₄. After the solvent was removed under reduced pressure, the residue was purified by flash chromatography (hexane/ethyl acetate, 1:1) to give the title compound (426 mg) as pale yellow crystals (y. 50%).

¹H-NMR (CDCl₃, 400 MHz) δ: 1.34 (6H, d, J=7 Hz), 2.24 (3H, s), 2.26 (3H, br s), 3.3-3.5 (5H, m), 7.09 (1H, br s), 7.19 (1H, s), 7.54 (1H, d, J=8 Hz), 7.93 (1H, d, J=8 Hz), 8.05 (1H, s), 8.40 (1H, br s).

### (2) 6-Amino-[3-[2-[3-isopropyl-6-(trifluoromethyl)benzothiophen-2-yl]ethyl]-5-methyl]-benzisoxazole

6-Acetamido-[3-[2-[3-isopropyl-6-(trifluoromethyl)benzothiophen-2-yl]methyl]-5-methyl]benzisoxazole (326 mg, 0.708 mmol) was suspended in 1M HCl (3.0 mL) and AcOH (7.0 mL). The suspension was heated under reflux for 23 hours. Then reaction mixture was cooled to room temperature, poured into ice-cold water, and neutralized with 4M NaOH. After ethyl acetate was added to the mixture, the organic layer was washed with brine, dried over Na₂SO₄. After the organic solvent was removed under reduced pressure, the residue was purified by flash chromatography (hexane/ethyl acetate, 2:1) to give the title compound (201 mg) as brown crystals (y. 68%).

¹H-NMR (CDCl₃, 400 MHz) δ: 1.36 (6H, d, J=7 Hz), 2.15 (3H, s), 3.26 (2H, t, J=8 Hz), 3.3-3.5 (5H, m), 3.99 (2H, br s), 6.74 (1H, s), 7.09 (1H, s), 7.52 (1H, d, J=8 Hz), 7.93 (1H, d, J=8 Hz), 8.05 (1H, s).

### (3) 6-Hydroxy-[3-[2-[3-isopropyl-6-(trifluoromethyl)benzothiophen-2-yl]ethyl]-5-methyl]-benzisoxazole

6-Amino-[3-[2-[3-isopropyl-6-(trifluoromethyl)benzothiophen-2-yl]ethyl]-5-methyl]-benzisoxazole (100 mg, 0.239 mmol) was suspended in 25% H₂SO₄ (2.0 mL). A solution of NaNO₂ (25 mg, 0.36 mmol) in water (1.0 mL) was added to the suspension while cooled on ice. After the stirred for 20 min under the same conditions, the mixture was added dropwise to 75% H₂SO₄ (1.5 mL) that heated to 120° C. The mixture was refluxed for 1 h at 120° C., cooled to room temperature, and poured into ice-cold water. After ethyl acetate was added to the mixture, the organic layer was washed with brine and dried over Na₂SO₄. After the solvent was removed, the residue was purified by flash chromatography (hexane/ethyl acetate, 5:1) to give the title compound (20 mg) as brown crystals (y. 20%).

¹H-NMR (CDCl₃, 400 MHz) δ: 1.36 (6H, d, J=7 Hz), 2.23 (3H, s), 3.2-3.5 (5H, m), 6.94 (1H, s), 7.37 (1H, s), 7.53 (1H, d, J=8 Hz), 7.93 (1H, d, J=8 Hz), 8.04 (1H, s).

### (4) Ethyl[3-[2-[3-isopropyl-6-(trifluoromethyl)benzothiophen-2-yl]ethyl]-5-methyl-benzisoxazol-6-yloxy]acetate

6-Hydroxy-[3-[2-[3-isopropyl-6-(trifluoromethyl)benzothiophen-2-yl]ethyl]-5-methyl]-benzisoxazole (528 mg, 1.26 mmol) and potassium carbonate (261 mg, 1.87 mmol) was suspended in acetone (10.0 mL). Ethyl bromoacetate (0.21 mL, 1.89 mmol) was added to the suspension while cooled on ice. The mixture was refluxed for 4 h, cooled to room temperature, and poured into ice-cold water. After the ethyl acetate was added to the mixture, the organic layer was washed with brine and dried over Na₂SO₄. After the solvent was removed, the residue was purified by flash chromatography (hexane/ethyl acetate, 100:1-5:1) to give the title compound (421 mg) as pale yellow crystals (y. 66%).

¹H-NMR (CDCl₃, 400 MHz) δ: 1.26 (3H, t, J=7 Hz), 1.35 (6H, d, J=7 Hz), 2.27 (3H, s), 3.2-3.5 (5H, m), 4.27 (211, q. J=7 Hz), 4.71 (2H, s) 6.72 (1H, s), 7.18 (1H, s), 7.52 (1H, d, J=8 Hz), 7.93 (1H, d, J=8 Hz), 8.18 (1H, s).

### [3-[2-[3-Isopropyl-6-(trifluoromethyl)benzothiophen-2-yl]ethyl]-5-methylbenzisoxazol-6-yloxy]acetic acid

Ethyl[3-[2-[3-isopropyl-6-(trifluoromethyl)benzothiophen-2-yl]ethyl]-5-methyl-benzisoxazol-6-yloxy]acetate (421 mg, 0.833 mmol) was dissolved in EtOH (5.1 mL). 1M NaOH (1.7 mL) was added to the solution, and the mixture was stirred for 20 h. Ice was added to the reaction mixture. The mixture was acidified with 1M HCl (3.4 mL), extracted with ethyl acetate. The organic layer was washed with brine, dried over Na₂SO₄. After the solvent was removed, the residue was purified by flash chromatography (CHCl₃/MeOH, 100:1-5:1) to give the title compound (370 mg) as pale yellow crystals.

MP (dec): 222-224° C.; FAB-MS (m/e):478 (M+1).

¹H-NMR (CDCl₃, 400 MHz) δ: 1.36 (6H, d, J=7 Hz), 2.27 (3H, s), 3.2-3.5 (5H, m), 4.78 (2H, s) 6.87 (1H, s), 7.23 (1H, s), 7.52 (1H, d, J=8 Hz), 7.93 (1H, d, J=8 Hz), 8.05 (1H, s).

### 1C. [3-[2-[3-Isopropyl-6-(trifluoromethyl)benzothiophen-2-yl]ethyl]-5-methylbenzisoxazol-6-yl]propionic acid

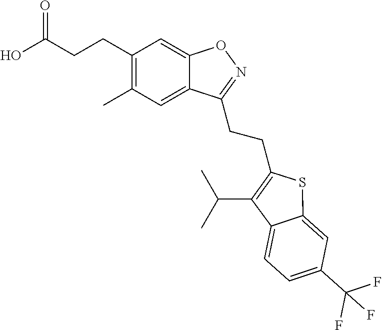

### (1) Methyl 2-bromo-[3-[2-[3-isopropyl-6-(trifluoromethyl)benzothiophen-2-yl]ethyl]-5-methylbenzoisoxazol-6-yl]propionate

6-Amino-[3-[2-[3-isopropyl-6-(trifluoromethyl)benzothiophen-2-yl]ethyl]-5-methyl]-benzisoxazole (150 mg, 0.358 mmol) was dissolved in methanol (1 mL)-acetone (2 mL). The solution was cooled to 0° C. 48% HBr (0.17 mL, 1.4 mmol) was dropwise added to the solution for 1 minute. A solution of NaNO₂ (30 mg, 0.43 mmol) in water (1.0 mL) was further added to the solution. The mixture was stirred for 30 minutes at 0° C. The mixture was raised to room temperature. Methyl acrylate (0.23 mL, 2.5 mmol) and copper(I) oxide (5.0 mg) were added to the mixture. The resulting mixture was stirred at 40° C. for 30 minutes. Ice-cold water was added to the mixture. The mixture was neutralized with ammonia solution, and extracted with ethyl acetate. The organic layer was washed with brine, and dried over Na₂SO₄. After the solvent was removed, the residue was purified by flash chromatography (hexane/ethyl acetate, 5:1) to give the title compound (135 mg) as pale yellow crystals (y. 66%).

¹H-NMR (CDCl₃, 400 MHz) δ: 1.33 (6H, d, J=7 Hz), 2.33 (3H, s), 3.3-3.4 (4H, m), 3.47 (2H, dd, J=5 Hz, 8 Hz), 3.57 (1H, dd, J=5 Hz, 8 Hz), 3.75 (3H, s), 4.42 (1H, t, J=8 Hz), 7.23 (1H, s), 7.37 (1H, s), 7.52 (1H, d, J=8 Hz), 7.92 (1H, d, J=8 Hz), 8.05 (1H, s).

### (2) Methyl[3-[2-[3-isopropyl-6-(trifluoromethyl)benzothiophen-2-yl]ethyl]-5-methyl-benzoisoxazol-6-yl]acrylate

Methyl 2-bromo-[3-[2-[3-isopropyl-6-(trifluoromethyl)benzothiophen-2-yl]ethyl]-5-methylbenzisoxazol-6-yl]propionate (740 mg, 1.30 mmol) was dissolved in MeOH (15 mL). Et₃N (2.6 mL, 2.63 mmol) was added to the solution, and the mixture was refluxed for 6 h. Ice was added to the reaction mixture. The mixture was acidified with 2M HCl, extracted with ethyl acetate. The organic layer was washed with brine, dried over Na₂SO₄. After the solvent was removed under reduced pressure, the crude crystal was filtered, to give the title compound (600 mg) as yellow crystal (y. 95%).

¹H-NMR (CDCl₃, 400 MHz) δ: 1.35 (6H, d, J=7 Hz), 2.41 (3H, s), 3.3-3.4 (3H, m), 3.48 (2H, dd, J=5 Hz, 8 Hz), 3.83 (3H, s), 6.44 (1H, d, J=16 Hz), 7.27 (1H, s), 7.53 (1H, d, J=8 Hz), 7.69 (1H, s), 7.93 (1H, d, J=8 Hz), 7.99 (1H, d, J=16 Hz), 8.05 (1H, s).

### (3) [3-[2-[3-Isopropyl-6-(trifluoromethyl)benzothiophen-2-yl]ethyl]-5-methylbenzisoxazol-6-yl]acrylic acid

Methyl[3-[2-[3-isopropyl-6-(trifluoromethyl)benzothiophen-2-yl]ethyl]-5-methyl-benzisoxazol-6-yl]acrylate (600 mg, 1.23 mmol) was dissolved in MeOH (3.0 mL)-THF (3.0 mL). A solution of NaOH (98 mg) in water (3.0 mL) was added to the solution, and the mixture was stirred for 24 h. The mixture was acidified with 2M HCl (2.5 mL), extracted with ethyl acetate. The organic layer was washed with brine, dried over Na₂SO₄. After the solvent was removed, dried to give the title compound (370 mg) as yellow crystals.

¹H-NMR (CDCl₃, 400 MHz) δ: 1.35 (6H, d, J=7 Hz), 2.42 (3H, s), 3.3-3.4 (3H, m), 3.49 (2H, dd, J=5 Hz, 8 Hz), 6.47 (1H, d, J=16 Hz) 7.28 (1H, s), 7.53 (1H, d, J=8 Hz), 7.74 (1H, s), 7.93 (1H, d, J=8 Hz), 8.05 (1H, s), 8.09 (1H, d, J=16 Hz).

### [3-[2-[3-Isopropyl-6-(trifluoromethyl)benzothiophen-2-yl]ethyl]-5-methylbenzisoxazol-6-yl]propionic acid

[3-[2-[3-Isopropyl-6-(trifluoromethyl)benzothiophen-2-yl]ethyl]-5-methylbenzisoxazol-6-yl]acrylic acid (555 mg, 1.17 mmol) was dissolved in EtOH (56 mL). Hydrazine monohydrate (1.14 mL, 23.4 mmol) was added to the solution, and the mixture was refluxed for 24 h under the oxygen atmosphere. The mixture was acidified with 2M HCl (35 mL), extracted with ethyl acetate. The organic layer was washed with brine, dried over Na₂SO₄. After the solvent was removed, the residue was purified by flash chromatography (CHCl₃/MeOH, 100:1-5:1) to give the title compound (440 mg) as pale yellow crystals (y. 79%).

MP: 178-180° C.; FAB-MS (m/e):476 (M+1).

¹H-NMR (CDCl₃, 400 MHz) δ: 1.35 (6H, d, J=7 Hz), 2.32 (3H, s), 2.71 (2H, t, J=7 Hz), 3.07 (2H, t, J=7 Hz), 3.32 (2H, dd, J=7 Hz, 8 Hz), 3.3-3.5 (1H, m), 3.47 (2H, dd, J=7 Hz, 8 Hz), 7.25 (1H, s), 7.36 (1H, s), 7.52 (1H, d, J=8 Hz), 7.93 (1H, d, J=8 Hz), 8.04 (1H, s), IR(KBr)cm⁻¹:2975, 2929, 1702, 1436, 1328, 1303, 1259, 1234, 1213, 1162, 1153, 1116, 1083, 883, 869, 815, 721, 418.

### Example 2

### Analysis of Tibialis-Anterior Muscles from the Rat Treated with Compound I.1

The muscle fibers on a set of animals that were treated with the compound 1.1 at the doses of 3 or 10 mg/kg once a day for 3 weeks are analyzed. Tibialis-anterior from 3 animals per group were used for both morpho-histology analysis and molecular analysis of specific markers of mitochondrial activities. The histology analysis was preformed on cryosection followed by a histochemical staining for succinate dehydrogenase (SDH) (Luquet S. et al., FASEB J, 2003. 17(15): p. 2299-301). Two biopsies of each muscle samples were generated and analyzed.

The quantification was performed by using Adobe Photoshop software (Lehr et al. Histochem Cytochem, 1997. 45(11): p. 1559-65, J Histochem Cytochem, 1999. 47(1): p. 119-26), and was based on pictures (in TIFF format) obtained from different slides of muscle (Tibialis-Anterior).

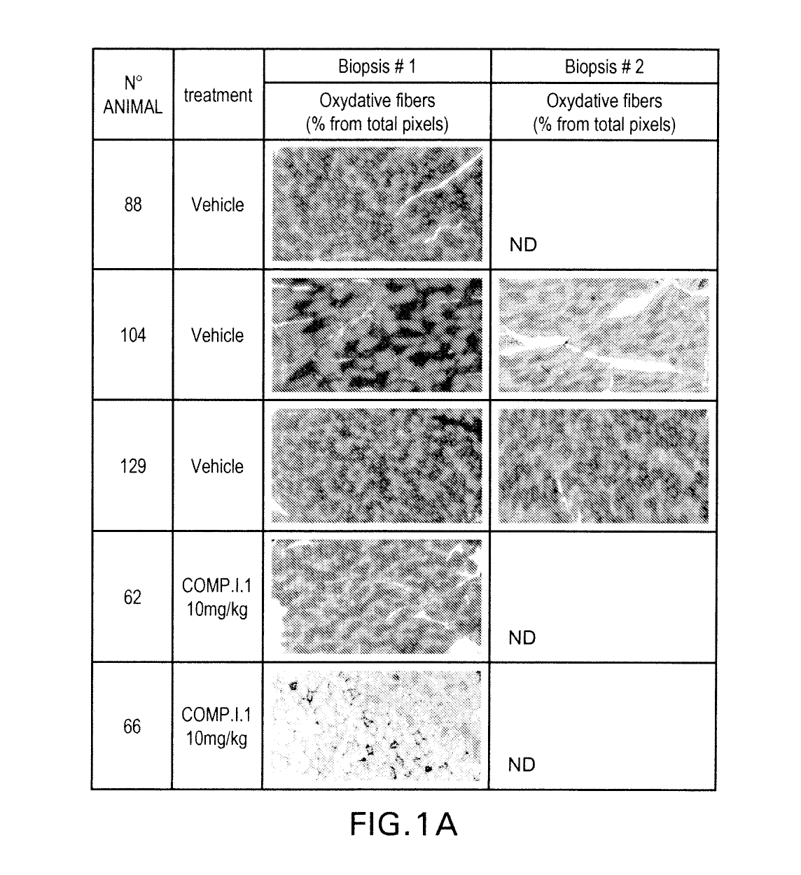

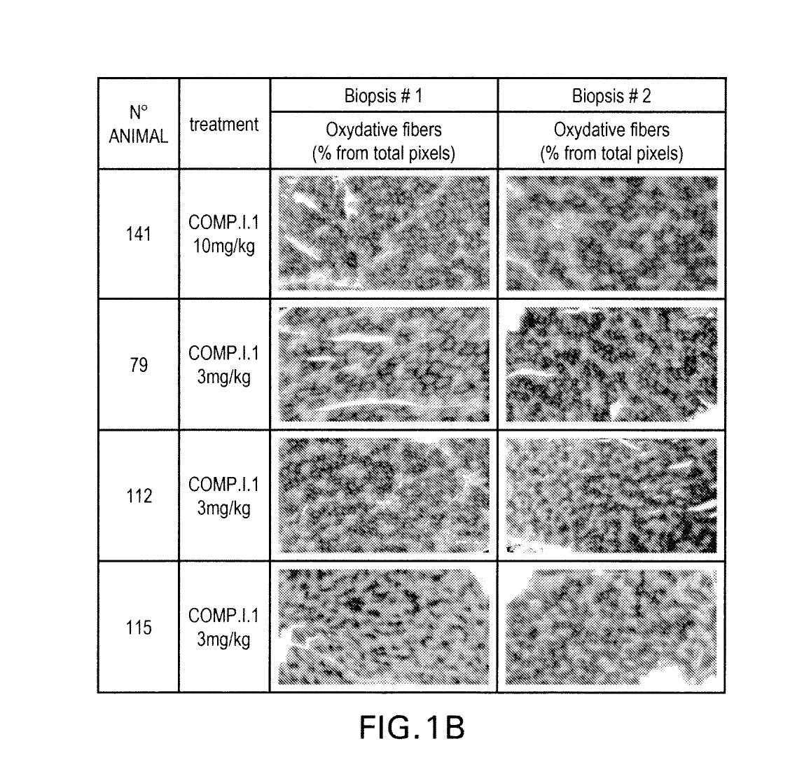

For each picture identical areas of about 57000 pixels at similar magnification were selected and the surface occupied by the stained fibers was measured (FIG. 1). The results are expressed as the percentage of the area of highly-stained and moderately-stain fibers from the total area selected. The analysis is performed in a blinded fashion.

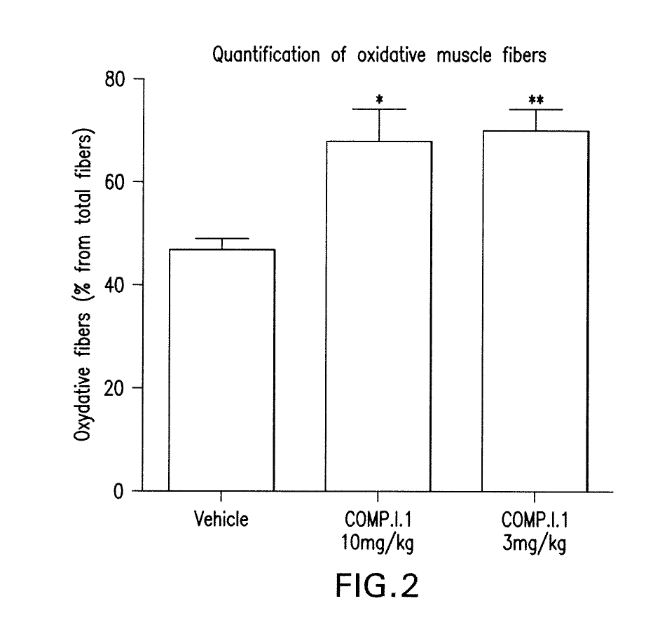

The results are represented in FIG. 2 and the data are presented in Table 8. Statistical analysis (Prism-Graphpad) was performed using unpaired T-test with: * corresponding to p<0.05 and ** to p<0.01.

TABLE 8
Biopsy data following treatment

| | | | | | | Biopsis #1 | Biopsis #2 |
|---|---|---|---|---|---|---|---|
| | | Biopsis #1 | | Biopsis #2 | | oxydative | oxydative |
| | | Non- | | Non- | | fibers | fibers |
| | | oxydative | oxydative | oxydative | oxydative | (% from | (% from |
| N^{o} | | fibers | fibers | fibers | fibers | total | total |
| ANIMAL | Treatment | (pixels) | (pixels) | (pixels) | (pixels) | pixels) | pixels) |
| 88 | Vehicle | 29999 | 26744 | ND | ND | 47.13 | ND |
| 104 | Vehicle | 31836 | 23700 | 26204 | 30647 | 42.68 | 53.91 |
| 129 | Vehicle | 29795 | 28233 | 33376 | 24652 | 48.65 | 42.48 |
| 62 | COMP.I.1 10 mg/kg | 18992 | 35030 | ND | ND | 68.84 | ND |
| 66 | COMP.I.1 10 mg/kg | 27170 | 30218 | ND | ND | 52.66 | ND |
| 141 | COMP.I.1 10 mg/kg | 16958 | 41070 | 9243 | 45326 | 70.78 | 83.06 |
| 79 | COMP.I.1 3 mg/kg | 15343 | 42685 | 16208 | 41820 | 73.56 | 72.07 |
| 112 | COMP.I.1 3 mg/kg | 16157 | 41871 | 1109 | 47009 | 72.16 | 81.01 |
| 115 | COMP.I.1 3 mg/kg | 27185 | 28767 | 16402 | 38128 | 51.41 | 69.92 |

### Example 3

### PPAR Selectivity of the Provided Compounds. Assays with GAL4-PPAR Chimera Receptors

Receptor Expression Plasmids

An established chimeric receptor system (1) was utilized to allow comparison of the relative transcriptional activity of the receptor subtypes. The mammalian expression vectors pSG5-GAL4-hPPARα, pSG5-GAL4-hPPARγ and pSG5-GAL4-hPPARδ, which express the ligand binding domains (LBDs) of human PPARα (amino acids 167-468), PPARγ1 (amino acids 176-477) and PPARδ (amino acids 139-441) each fused to the yeast transcription factor GAL4 DNA binding domain (amino acid 1-147), were used. MH100×4-tk-luc (2) was used as the reporter plasmid.

Assays with Full-Length Receptors

Receptor expression plasmids: Full-length coding sequences for human PPARα, PPARγ and PPARδ were inserted into the mammalian expression vector pcDNA3.1+ or pCMX. The full-length coding sequence for human RXRα was inserted into pcDNA3.1+.

Reporter plasmid: PPREx3-tk-luc (3) was used as the reporter plasmid. It contains three copies of rat Acyl-CoA oxidase peroxisome proliferator response element (PPRE) sequence (AGGACA-A-AGGTCA).

Transient Transfection Assays

The African green monkey kidney cell line, CV-1 was used for the transfection assays. CV-1 cells were seeded in 24-well plates at 0.5×10⁵ cells per well and were cultured for 24 hours. Transfection mixtures for chimera receptors contained 30 ng of receptor expression plasmid, 120 ng of the reporter plasmid, 350 ng of pCMX-β-galactosidase (βGAL) expression plasmid as a control for transfection efficiency, 250 ng of pGEM4 carrier plasmid and 2 μL of a lipofection reagent (Lipofectamine 2000, Invitrogen). Transfection mixtures for full-length receptors contained 15 ng of receptor expression plasmid, 15 ng of hRXRα expression plasmid, 120 ng of the reporter plasmid, 350 ng of pCMX-βGAL, 250 ng of pGEM4 and 2 μL of Lipofectamine 2000. These mixtures were added to cells and incubated for 5 hours according to the manufacturer's instructions. After the transfection, cells were incubated for an additional 40 hours in the presence of the compound I.1 or each reference compound. Cell lysates were prepared with a lysis buffer (Passive Lysis Buffer, Promega) and used in the luciferase and βGAL assays. The luciferase and βGAL activity were measured according to the methods of Umesono, K. et al. (4) with slight modifications. A substrate reagent kit (Picagene, Toyo Ink) was used for the luciferase assay. Assays were performed in duplicate and triplicate for GAL4-chimeras and full-length receptors, respectively. Experiments were repeated at least four times.

Calculation of Relative PPAR Transactivation Activities

Each point of a relative PPAR transcriptional activity to maximal activity was calculated based on the values bellow:

Luciferase activity of cells treated with a positive control (10⁻⁶ M GW-590735 for hPPARα, 3×10⁻⁶ M rosiglitazone maleate for hPPARγ assays and 10⁻⁷ M GW-501516 for hPPARδ) as the maximal activity, and luciferase activity of cells treated with 0.1% of DMSO as the minimum activity.

Calculation of EC₅₀ Values

EC₅₀ values defined as the concentration of the compound I.1 and GW-501516 to produce 50% of maximal reporter activity were calculated with Prism software (Graphpad Software).

TABLE 9
PPAR Selectivity Data for Selected Compounds provided in Cell-Based
Transactivation Assays

| | | Transactivation activity | | | Transactivation activity | | | |
|---|---|---|---|---|---|---|---|---|
| | | GAL 4 DBD-hPPAR | | | FULL-hPPAR + mRXRalpha | | | |
| | Molecular | LBD (EC50-Um) | | | (EC50-Um) | | | |
| Type | Weight | alpha | gamma | delta | alpha | gamma | delta | Structure |
| δ | 491.52 | >10 | >8 | 0.018 | 3.2 | 5.8 | 0.0022 | 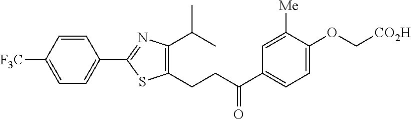 |
| δ | 457.91 | >10 | >10 | 0.032 | 3.8 | 9.8 | 0.058 | 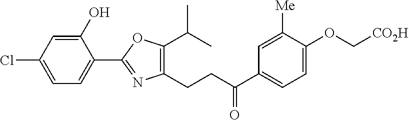 |
| δ | 489.35 | >10 | >10 | 0.025 | 7.8 | >10 | 0.007 | 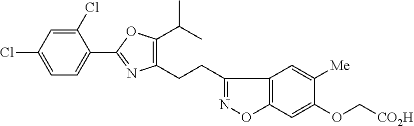 |
| δ | 489.52 | >10 | >10 | 0.045 | >10 | >10 | 0.019 | 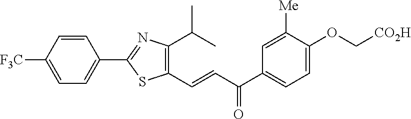 |
| δ | 507.58 | 3.3 | 6.7 | 0.018 | | | | 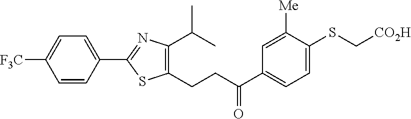 |
| δ | | >10 | >10 | >1 | >8 | >8 | >0.3 | 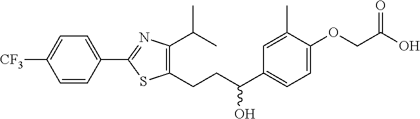 |
| δ | | >10 | >10 | >0.7 | | | | 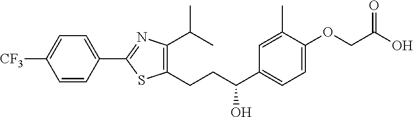 |

### Example 4

### Mouse and Rat PPAR Transcriptional Activation by the Compound I.1 in Cell-Based Transactivation Assay

Assays with GAL4-PPAR Chimera Receptors.

Receptor Expression Plasmids

An established chimeric receptor system (Lehmann J M et. al. J Biol Chem. 1997; 272(6):3406-10) was utilized to allow comparison of the relative transcriptional activity of the receptor subtypes. The mammalian expression vectors pSG5-GAL4-PPARα, pSG5-GAL4-PPARγ and pSG5-GAL4-PPARδ from homo sapiens (hs) (NM_{—}006238, NM_{—}015869, NM_{—}001001928), macaca mulatta (mm) (NM_{—}001033029, XM_{—}001116676, NM_{—}001032860), mus musculus (m) (NM_{—}011144, U10375, NM_{—}011146) and rattus norvegicus (r) (NM_{—}013141, NM_{—}013196, NM_{—}013124), which express the ligand binding domains (LBDs) of human PPARα (amino acids 167-468 for hs and m, amino acids 167-467 for mm), PPARγ1 (amino acids 176-477 for hs, amino acids 204-505 for m and mm from PPARγ) and PPARδ (amino acids 139-441 for hs and mm, amino acids 139-440 for m) each fused to the yeast transcription factor GAL4 DNA binding domain (amino acid 1-147) and the human glucocorticoid receptor (amino acids 1-76), were cloned and produced by Euromedex and Genscript.

Reporter plasmid: MH100×4-tk-luc (Forman B M et al. Cell. 1995 81(4):541-50) was used as the reporter plasmid.

Transient Transfection Assays

The African green monkey kidney cell line, CV-1 was used for the transfection assays. CV-1 cells were seeded in 24-well plates at 0.5×10⁵ cells per well and were cultured for 24 hours. Transfection mixtures for chimera receptors contained 30 ng of receptor expression plasmid, 120 ng of the reporter plasmid, 350 ng of pCMX-β-galactosidase (βGAL) expression plasmid as a control for transfection efficiency, 250 ng of pGEM4 carrier plasmid and 2 μL of a lipofection reagent (Lipofectamine 2000, Invitrogen). These mixtures were added to cells and incubated for 5 hours according to the manufacturer's instructions. After the transfection, cells were incubated for an additional 40 hours in the presence of the compound I.1 or each reference compound at different concentrations. Cell lysates were prepared with a lysis buffer (Passive Lysis Buffer, Promega) and used in the luciferase and βGAL assays. The luciferase and βGAL activity were measured with the Luciferase assay system (E4030, Promega) and with the βGAL enzyme assay system (E2000, Promega). Assays were performed in triplicate for GAL4-chimeras. Experiments were repeated at least three times.

Calculation of Relative PPAR Transactivation Activities

Each point of a relative PPAR transcriptional activity to maximal activity was calculated based on the values bellow:

Luciferase activity of cells treated with a positive control (10⁻⁵ M GW-590735 for hPPARα, 3×10⁻⁵ M rosiglitazone maleate for hPPARγ assays and 10⁻⁵ M GW-501516 for hPPARδ) as the maximal activity, and luciferase activity of cells treated with 0.1% of DMSO as the minimum activity.

Calculation of EC₅₀ Values.

EC₅₀ values defined as the concentration of the compound I.1 and GW-501516 to produce 50% of maximal reporter activity were calculated with Prism software (Graphpad Software).

Experiments Runned with 10% Serum vs 0.1% Serum.

We runned the experiments at 2 serum concentrations: 10% and 0.1%. The EC50 were calculated as previously described.

We first set-up the assay and we validate the GAL4-chimeras/reporter plasmids by using homo-sapiens sequences of the different PPAR. The experiments were conducted at 10% and 0.1% serum with GW501516 (PPARδ agonist).

TABLE 10
Human PPAR transactivation activities of GW-501516 in cell-based
transactivation assays at 0.1% serum

| | | GAL4-chimera | | GAL4-chimera | | |
|---|---|---|---|---|---|---|
| | | (human) | | (human) − 0.1% serum | | |
| Com- | | | EC₅₀ (μM) | | EC₅₀ (μM) | |
| pound | Structural formula | hPPARα | hPPARγ | hPPARα | hPPARγ | hPPARδ |
| GW- 501516 | 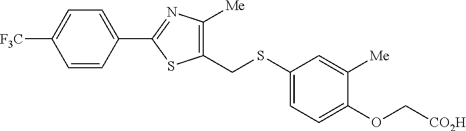 | 4.6 ± 1.9 | >10 | 1.02 ± 0.4 | 1.9 ± 0.4 | 0.0011 ± 0.0001 |

The transactivation activities of the compound I.1 and GW501516 are measured in rat and mouse transactivation assays at 10% and 0.1% serum (Tables 11 and 12).

TABLE 11
PPAR transactivation activities of the compound I.1 and GW-501516 in
cell-based transactivation assays at 10% serum

| | | GAL4-chimera (mouse) | | | GAL4-chimera (rat) | | |
|---|---|---|---|---|---|---|---|
| | | | EC₅₀ (μM) | | | EC₅₀ (μM) | |
| Compound | Structural formula | hPPARα | hPPARγ | hPPARδ | hPPARα | hPPARγ | hPPARδ |
| COMP.I.1 | 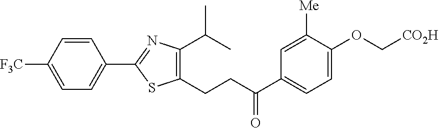 | >10 | >10 | 0.327 ± 0.07 | >10 | >10 | 0.692 ± 0.177 |
| GW-501516 | 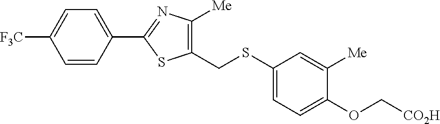 | >10 | >10 | 0.154 ± 0.007 | >10 | >10 | 0.427 ± 0.29 |

TABLE 12
PPAR transactivation activities of the compound I.1 and GW-501516 in
cell-based transactivation assays at 0.1% serum

| | | GAL4-chimera (mouse) | | | GAL4-chimera (rat) | | |
|---|---|---|---|---|---|---|---|
| | | | EC₅₀ (μM) | | | EC₅₀ (μM) | |
| Compound | Structural formula | hPPARα | hPPARγ | hPPARδ | hPPARα | hPPARγ | hPPARδ |
| COMP.I.1 | 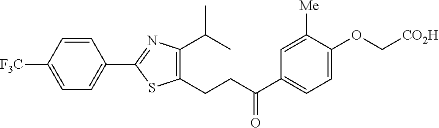 | >10 | >10 | 0.065 ± 0.001 | >10 | >10 | 0.118 ± 0.031 |
| GW-501516 | 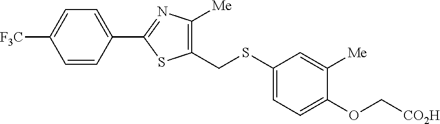 | 1.1 ± 0.1 | >10 | 0.018 ± 0.003 | 1.96 ± 0.7 | >10 | 0.072 ± 0.025 |

Plasmid Reference:

MH100×4-tk-luc plasmid: pGL3-basic luciferase reporter gene vector (Promega, Cat#E1751) (Amp^{R}); Insert: UAS_{G}X4 and TK. Minimal TK promoter (162 bp) is cloned upstream luciferase coding sequence. The GAL4 binding sequence UAS_{G}×4(5′-CGACGGAGTACTGTCCTCCGAGCT, four copies) (SEQ ID NO:1) is cloned upstream of the promoter.

(SEQ ID NO: 2)
CGACGGAGTACTGTCCTCCGAGCTCGACGGAGTACTGTCCTCCGAGC
TCGACGGAGTACTGTCCTCCGAGCTCGACGGAGTACTGTCCTCCGAG
CTTCTAGAGGATCCGGCCCCGCCCAGCGTCTTGTCATTGGCGAATTC
GAACACGCAGATGCAGTCGGGGCGGCGCGGTCCGAGGTCCACTTCGC
ATATTAAGGTGACGCGTGTGGCCTCGAACACCGAGCGACCCTGCAGC
GACCCCCTTAACAGCGTCAACAGCGTGCCGCAGATCTCGAGGAGCTT
GGCATTCCGGTACTGTTGGTAAA [symbol] [symbol] ---

Bold: 4×UAS_{G} elements
Italics: Thymidine kinase minimal sequence (V00470)
Bold Italics: beginning of luciferase

pSG5-GAL4-PPARLBD: pSG5 (Stratagene—Catalog#216201) (Amp^{R}) The glucocorticoid receptor (amino acids 1-76) is cloned upstream the GAL4 DNA binding domain. The PPAR sequence is cloned in 3′ of the GAL4 sequence.

(SEQ ID NO: 3)
ATGGACTCCAAAGAATCATTAACTCCTGGTAGAGAAGAAAACCCCAG
CAGTGTGCTTGCTCAGGAGAGGGGAGATGTGATGGACTTCTATAAAA
CCCTAAGAGGAGGAGCTACTGTGAAGGTTTCTGCGTCTTCACCCTCA
CTGGCTGTCGCTTCTCAATCAGACTCCAAGCAGCGAAGACTTTTGGT
TGATTTTCCAAAAGGCTCAGTAAGCAATGCGCAGCAGCCAAAGCTAC
TGTCTTCTATCGAACAAGCATGCGATATTTGCCGACTTAAAAAGCTC
AAGTGCTCCAAAGAAAAACCGAAGTGCGCCAAGTGTCTGAAGAACAA
CTGGGAGTGTCGCTACTCTCCCAAAACCAAAAGGTCTCCGCTGACTA
GGGCACATCTGACAGAAGTGGAATCAAGGCTAGAAAGACTGGAACAG
CTATTTCTACTGATTTTTCCTCGAGAAGACCTTGACATGATTTTGAA
AATGGATTCTTTACAGGATATAAAAGCATTGTTAACAGGATTATTTG
TACAAGATAATGTGAATAAAGATGCCGTCACAGATAGATTGGCTTCA
GTGGAGACTGATATGCCTCTAACATTGAGACAGCATAGAATAAGTGC
GACATCATCATCGGAAGAGAGTAGTAACAAAGGTCAAAGACAGTTGA
CTGTATCG [symbol] [symbol] ---

Bold: Glucocorticoid receptor (homo sapiens—amino acids 1-76);
Italics: GAL4 DNA binding domain (Saccharomyces Cerevisiae—amino acids 1-147);
Bold Italics: PPAR Ligand Binding Domain sequence.

### Example 5

### Hepatic and Peripheral Insulin Sensitive Effects in Rats Fed a High Fat/Medium Fructose Diet (WD), Alone or in Association with Metformin

After 8 weeks of high fat diet, rats were treated for 5 weeks with pioglitazone (10 mg/kg), metformin (50 mg/kg), the compound I.1 (3 and 10 mg/kg), alone or in association with metformin (50 mg/kg), and GW501516 (10 mg/kg). Body weight gain was decreased by metformin and this effect was emphasized when it was associated with the compound I.1 in a dose of 10 mg/kg (also confirmed by a reduction on adipose tissue mass measured at the end of the study). After 17 days of treatment, rats were fasted for 4 hours and blood was sampled. Pioglitazone, metformin (alone or in association with the compound I.1), decreased HOMA-IR by ˜50% and the compound I.1 in a dose of 10 mg/kg and GW501516 decreased it by ˜25%. Only pioglitazone improved plasma adiponectin levels. No treatment clearly affected plasma FFA and TG levels. The glucose tolerance was assessed after 21 days of treatment by an oral glucose load performed after 6 hours of fasting. The compound I.1 in a dose of 10 mg/kg significantly improved glucose tolerance in this model in a better extent than pioglitazone. After 5 weeks of treatment, an euglycemic-hyperinsulinemic clamp procedure was performed in awake rats. Two doses of insulin were infused with 3H-glucose: 0.2 U/kg/h to assess an effect on HGP and 0.8 U/kg/h to inhibit HGP and then to assess an effect on whole body glucose utilization. GIR was significantly increased during the first insulin level by pioglitazone (138%), metformin (112%), the compound I.1 in a dose of 3 mg/kg (105%) and the compound I.1 in a dose of 10 mg/kg+metformine (82%). The other treatments tended to increase GIR by at least 50%. The effect was less marked on GIR measured during the second level of insulin. All treatments had a tendency to increase GTO (10-22%), the effect being more marked during the second step (19-27%). In a general manner, the compound I.1 improved insulin resistance in a lesser extent than pioglitazone and metformin, but was more effective than GW501516. There were no real dose dependent effects. If pioglitazone decreased HGP and increased whole body glycogen synthesis, metformin and the compound I.1 tended to increase in addition whole body glucose oxidation. This last effect was slightly emphasized when the 2 drugs were associated. The compound I.1 did not affect TG content in the liver whereas pioglitazone and GW501516 tended to decrease it. The compound I.1 in a dose of 10 mg/kg associated with metformin and GW501516 tended to decreased TNF-α content in the liver although non significantly. All treatments decreased TNF-α content in EWAT, except pioglitazone. In conclusion, the compound I.1 has interesting effects on glucose homeostasis in WD fed rats by increasing glucose tolerance and improving insulin resistance at both the level of liver and peripheral tissues. A combination with metformin could emphasize the effects of the compound I.1 (Abbreviations: WD: Western Diet, FFA: Free Fatty Acids, TG: Triglycerides, AUC: Area Under the Curve; GIR: Glucose Infusion Rate, HGP: hepatic glucose production, TO: Turn Over, IWAT: Inguinal White Adipose Tissue, EWAT: Epididymal White Adipose Tissue, RWAT: Retroperitoneal White Adipose Tissue, PWAT: Perirenal White Adipose Tissue, SDH: Succinate DeHydrogenase).

Animal Model

Male SD rats (250-275 g) were first fed with the western diet during 8 weeks for inducing insulin resistance. The first set of rats was undergoing an OGTT after 3 weeks of treatment (half rats/group of the clamp arm) and the second set was also undergoing an OGTT after 3 weeks of treatment (half rats/group of clamp arm) and the Histology study (3 rats/group).

Rats of each set were screened and randomized into the several groups according to their fasted (4 h) plasma glucose, insulin levels (for HOMA-IR calculation) and body weight. Rats that did not respond to the western diet were excluded from the study.

Homogenous mild obese and insulin resistant rats were allocated into the different treatment groups and western diet was continued until the end of the experiment. Given that, the 2 series started with a gap of one week

Treatments

Dosage regimen: Rats were treated once daily via the oral route, in the morning. The duration of the treatment was between 5 and 5.5 weeks.

Test Groups

Group 1: vehicle (n=12)
Group 2: pioglitazone, 10 mg/kg (n=12)
Group 3: metformin, 50 mg/Kg (n=12)
Group 4: the compound I.1, 3 mg/Kg (n=12)
Group 5: the compound I.1, 10 mg/Kg (n=12)
Group 6: the compound I.1, 3 mg/Kg+metformin 50 mg/kg (n=12)
Group 7: the compound I.1, 10 mg/Kg+metformin 50 mg/kg (n=12)
Group 8: GW501216, 10 mg/Kg (n=12)

Fasting Conditions

Fasting conditions for the OGTT and the clamp procedure: food was removed from the cage and litter was changed just before light off (between 7:30 and 8:00 am). Then each experiment started after about 6 hours of fasting (between 1:30 and 2:00 pm).

Fasting conditions for plasma parameters: food was removed from the cage and litter was changed 4 hours after light off (between 11:30 am and noon). Then blood collection started after 4 hours of fasting (at 4:00 pm).

Oral Glucose Tolerance Test

After 3 weeks of treatment, rats were fasted for 6 hours and an oral glucose load (2.5 g/kg body weight) was performed. Blood glucose was measured 30 minutes before glucose load and at 0, 15, 30, 60, 90 and 120 minutes.

Euglycemic-Hyperinsulinemic 2 Steps Clamp with 3H-Glucose

After 4 weeks of treatment, a catheter was implanted into the femoral vein under isoflurane anaesthesia and a period of 5-6 days was respected for recovery. Rats that did not recover body weight were excluded from the study. The accepted body weight loss estimated the day of perfusion was fixed at 5% in groups where treatments should not affect body weight (as seen during body weight follow-up) (vehicle, pioglitazone and the compound I.1, mg/kg groups) and at 10% where treatments decreased it (the 5 other groups). The morning of the clamp procedure, rats were treated and fasted for 6 hours prior to tracer, glucose and insulin infusions. The beginning of the clamp procedure was performed in the middle of the dark cycle.

The euglycemic hyperinsulinemic 2 steps insulin clamp was performed using 0.2 U/kg/h and 0.8 U/kg/h insulin infusion associated with 3H-glucose infusion. We then assessed:

Whole body glucose utilization

Hepatic glucose production

Glucose infusion rate

Whole body glycogen and glycolytic rates

In Vivo Glucose Utilization Rate

During the clamp procedure, a bolus (30 μCi) of D-[3-3H]glucose was first injected followed by 4 μCi/min/kg infusion rate during all the experiment to ensure a detectable plasma D[3-3H]glucose enrichment. A bolus of insulin (100 mU) was first injected, and insulin was then infused at a rate of 0.2 U/kg/h for the first 2 hours and 0.8 U/kg/h from 120 minutes to 210 minutes.

Throughout the infusion, glycaemia was assessed with a blood glucose meter from the tip of the tail vein when needed. Euglycemia was maintained by periodically (every 10 minutes) adjusting a variable infusion of 30% glucose. Plasma glucose concentrations and D-[3-3H]glucose specific activity were determined during stable phase in 10 μl of blood sampled from the tip of the tail vein every 20 minutes from 60 to 120 minutes during the first step and from 150 to 210 minutes during the second step.

For glucose turnover measurements, D-[3-3H]glucose enrichments were determined from total blood after deproteinization by a Zn(OH)₂ precipitate. Briefly, an aliquot of the supernatant was evaporated to dryness to determine the radioactivity corresponding to D-3-3H. In a second aliquot of the same supernatant, glucose concentration was assessed by the glucose oxidase method (Biomérieux, France).

Calculation

Calculations for glucose turnover measurements were made from parameters obtained during the infusions in steady-state condition (60-120 and 160-210 minutes). Briefly, the D-[3-3H]glucose specific activity was calculated by dividing the D-[3-3H]glucose enrichment by the plasma glucose concentration. The whole body glucose turnover rate was calculated by dividing the rate of D[3-3H]glucose by the D-[3-3H]glucose plasma specific activity. For each rat, the mean values were calculated and averaged with values from rats of the same group. The whole body glycolysis rate was measured by assessing the amount of tritiated water accumulated in the blood during the 3H-glucose infusion and the whole body glycogen synthesis rate was calculated by the difference between the whole body glucose turnover and the whole body glycolysis rate.

Blood, Tissue Collection and Biochemistry

At the end of the clamp procedure (9.5 hours fasting), perirenal, retroperitoneal, epididymal and inguinal fat pads and liver weights were recorded. Blood was collected from cardiac puncture and plasmas were stored at −80° C. for further assays if needed (depending on the radioactive state of samples). Liver triglyceride content was assessed (enzymatic-colorimetric method) as well as liver and adipose tissue TNF-α content (ELISA method).

Deviations

Body weight measurements had to be performed between 9:00 and 11:00 am. The first set of rats was weighted at 11:15 am on day 25. The second set of rats was weighted at 8:30 on days 17 and 21, at 1:45 pm on day 24 and at 11:10 am on day 28. In the histology arm, the rat #66 was treated on day 17 with the compound I.1 in a dose of 10 mg/kg and in addition with the mix of the compound I.1 in a dose of 3 mg/kg+metformin 50 mg/kg.

Results and Discussion.

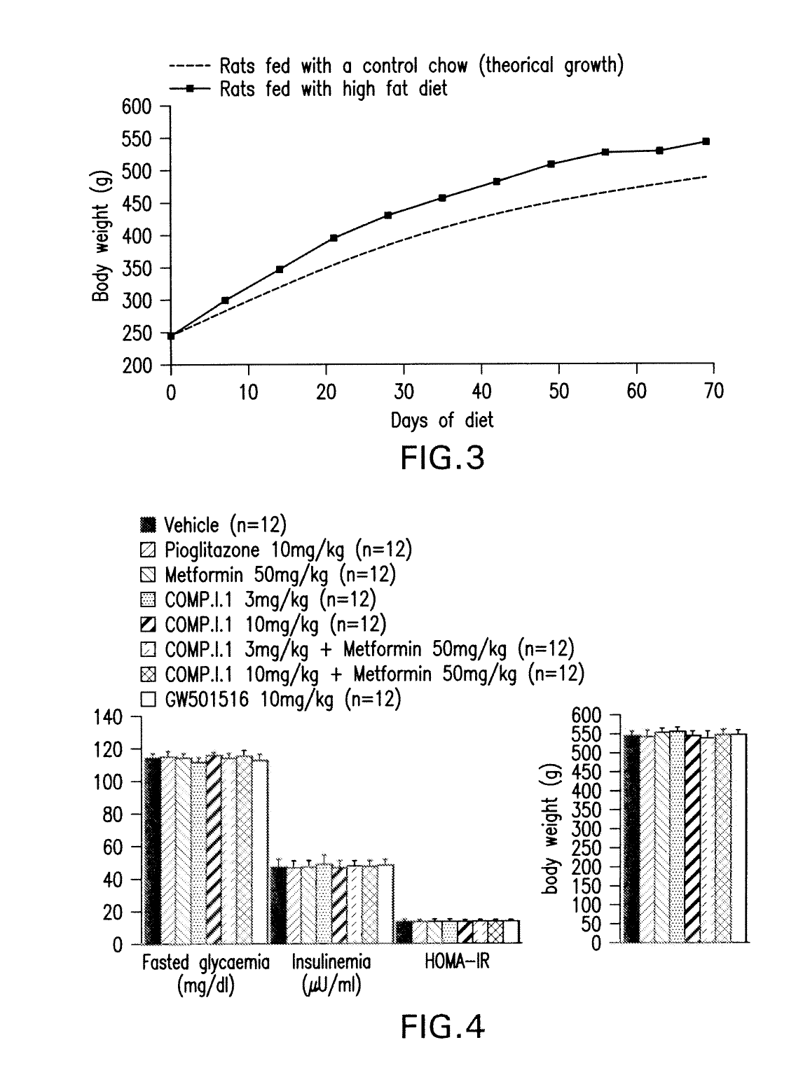

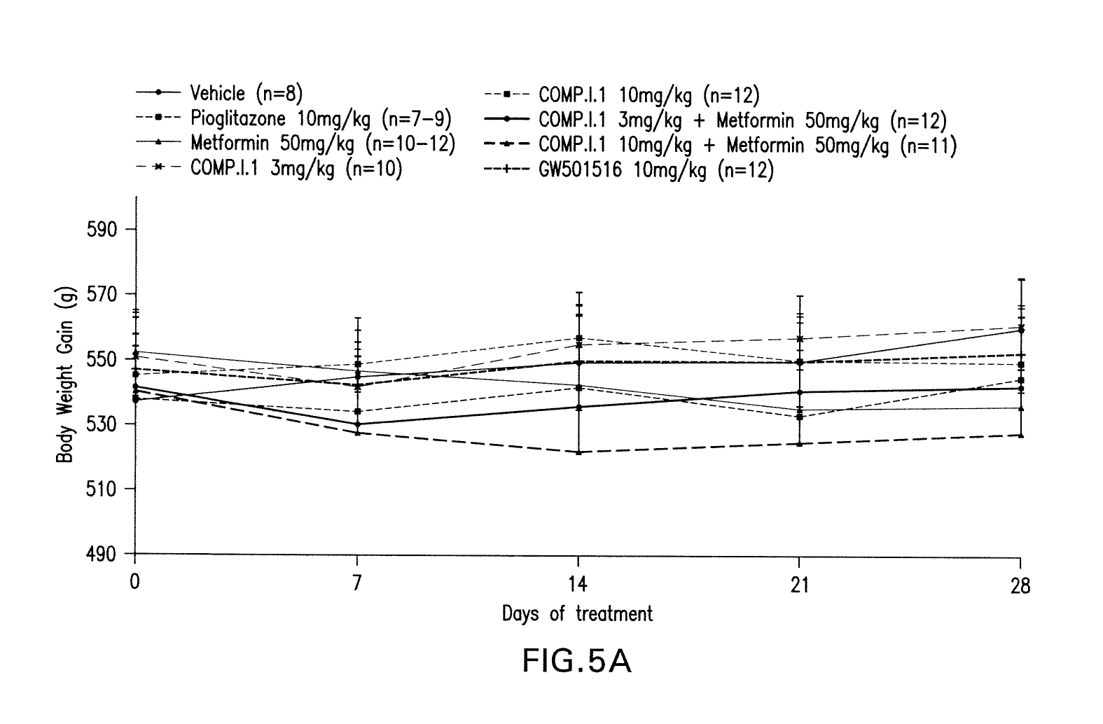

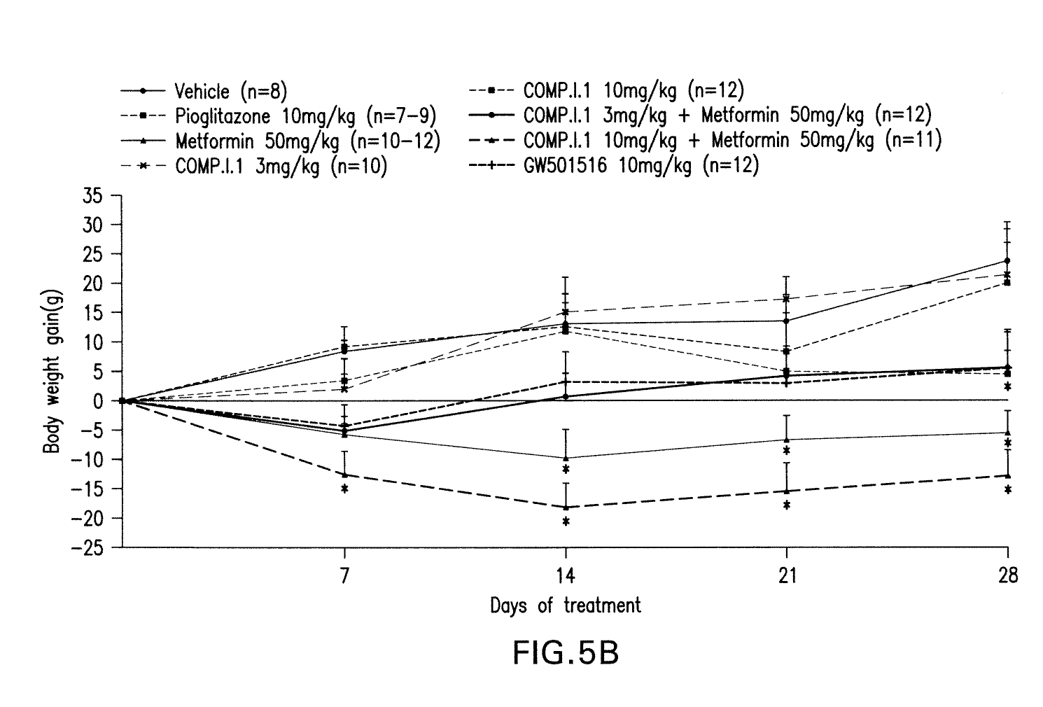

Animal model WD fed rats. After 8 weeks of WD, body weight gain was 296±4 g. Body weight of rats fed with a control chow generally reaches about 480 g at 16 weeks old (Charles River data). Rats used in this study reached 540 g±4 g at the same age (FIG. 3). So the model was mild obese (12.5% of difference). Insulinemia and blood glucose were measured. Mild obese and insulin resistant rats were homogeneously allocated in the 8 groups according to their fasted glycaemia (˜114 mg/dl), insulinemia (˜48 μU/ml), HOMA-IR (˜13) and body weight (˜547 g) (FIG. 4). Data of the histology arm are given in annex (n=3 per group). Body weight follow-up during treatment. FIG. 5A shows that the body weight of vehicle treated rats was increasing during the treatment period (+23.6±6.7 g after 4 weeks). Pioglitazone and the compound IA in a dose of 3 mg/kg did not affect body weight gain. Metformin associated with the compound I.1 in a dose of 3 mg/kg, and GW501516 slightly decreased body weight the first week of treatment but not in a significant manner. Growth of rats was then parallel to the vehicle group during 2 weeks. The body weight decreasing effect of the 2 compounds was significant after 4 weeks of treatment (FIG. 5A). The compound I.1 in a dose of 10 mg/kg had no effect the first 2 weeks of treatment and decreased thereafter body weight gain, until a significant effect after 4 weeks of treatment (FIG. 5B). After one week of treatment, body weight was significantly decreased in rats treated with metformin associated with the compound I.1 in a dose of 10 mg/kg. Metformin alone significantly decreased body weight after 2 weeks of treatment. After these 2 first weeks, body weight gain slightly increased in these 2 groups, but it remained significantly lower compared to the vehicle group (FIG. 5B).

Plasma Biochemistry after 17 Days of Treatment

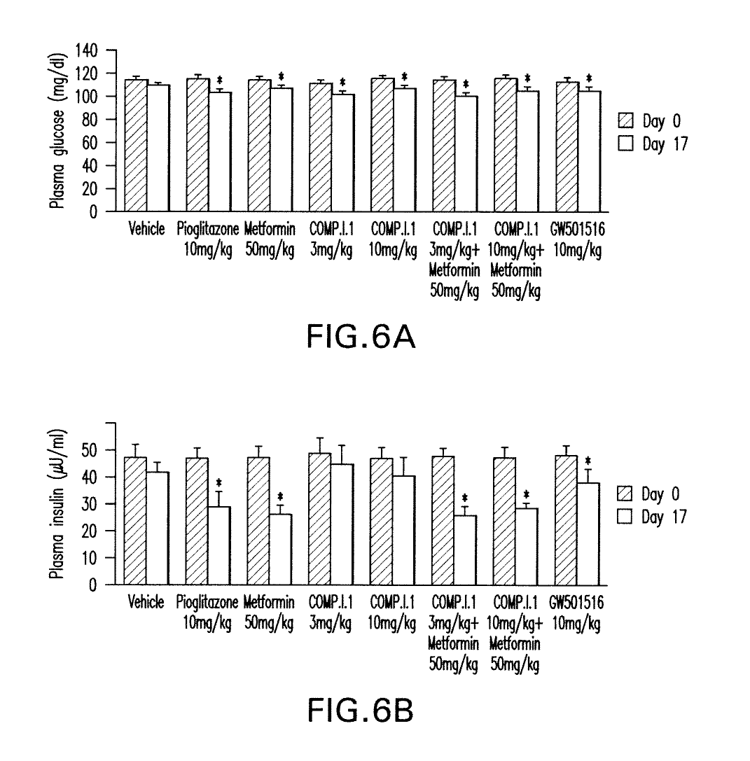

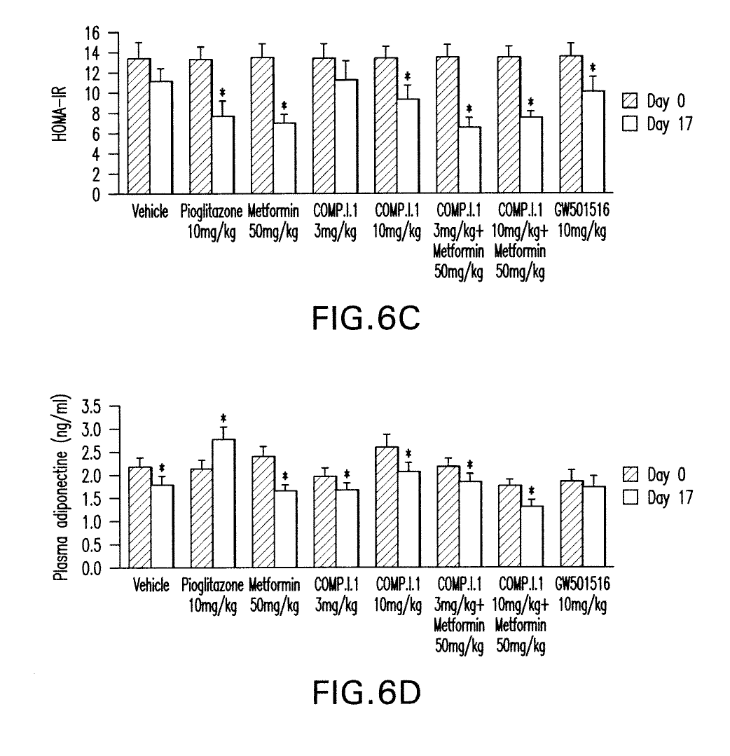

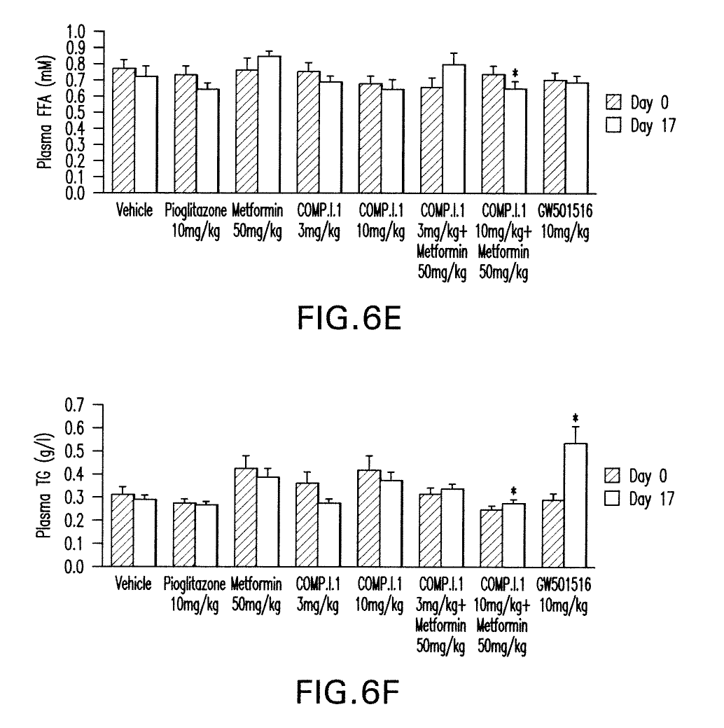

Rats were fasted 4 hours before blood glucose measurement and blood collection. Biochemical parameters were assessed in plasma. All treatments significantly decreased glycemia (6-11%, FIG. 6A). Pioglitazone significantly decreased plasma insulin by 39%. Metformin, alone or in association with the compound I.1 in a dose of 3 or 10 mg/kg, significantly decreased plasma insulin by 44, 46 and 40% respectively. GW501516 significantly decreased it by 21% (FIG. 6B). So the effects observed on glycemia and plasma insulin had direct repercussion on HOMA-IR and it appears that the compound I.1 in a dose of 10 mg/kg significantly decreased HOMA-IR by 28% (FIG. 6C). Plasma adiponectin was significantly decreased by 18% in the vehicle group after 17 days of treatment. As expected, pioglitazone significantly increased plasma adiponectin levels. GW501516 prevented the decrease in circulating adiponectin. In all other groups, adiponectin decreased by 15-30% (FIG. 6D). So metformin and the compound I.1, alone or in association, did not prevent a decrease in plasma adiponectin in this model. There was no significant effect of the treatments on plasma FFA and TG levels, except metformin associated with the compound I.1 in a dose of 10 mg/kg that significantly decreased plasma FFA by 12% (FIG. 6E) and increased plasma TG by 11% (FIG. 6F). But these effects are not physiologically relevant. On the other hand, GW501516 significantly increased plasma TG (by 84%) (FIG. 4F).

Glucose Tolerance Test after 3 Weeks of Treatment

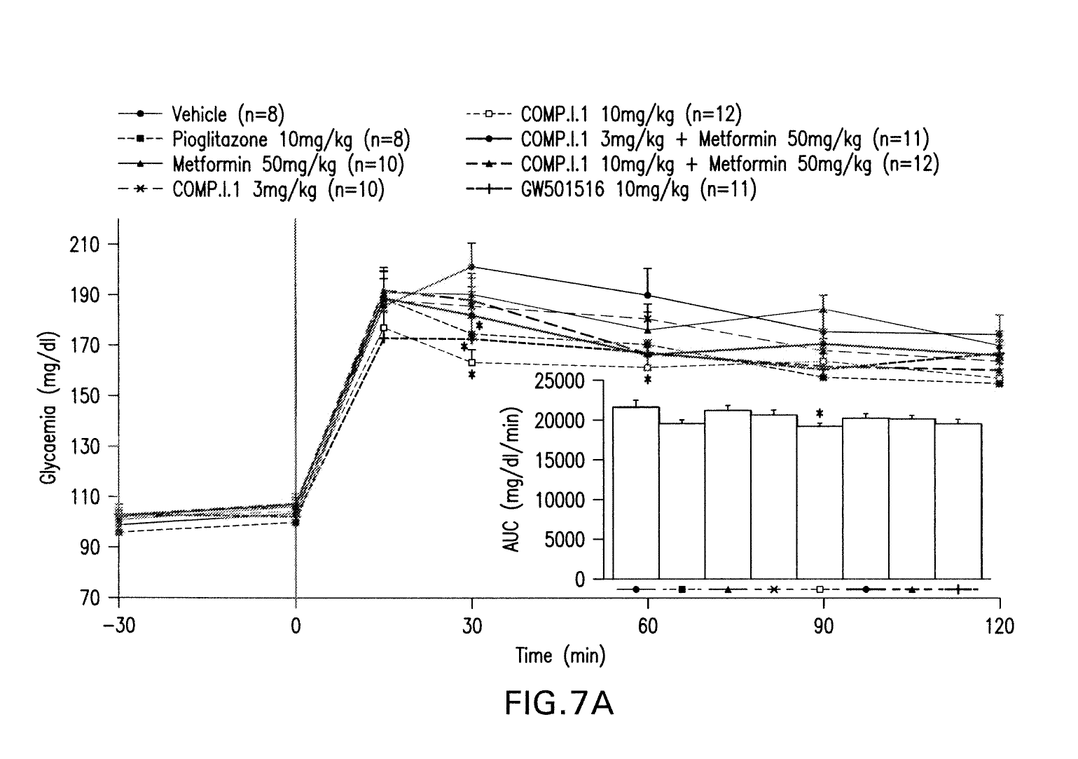

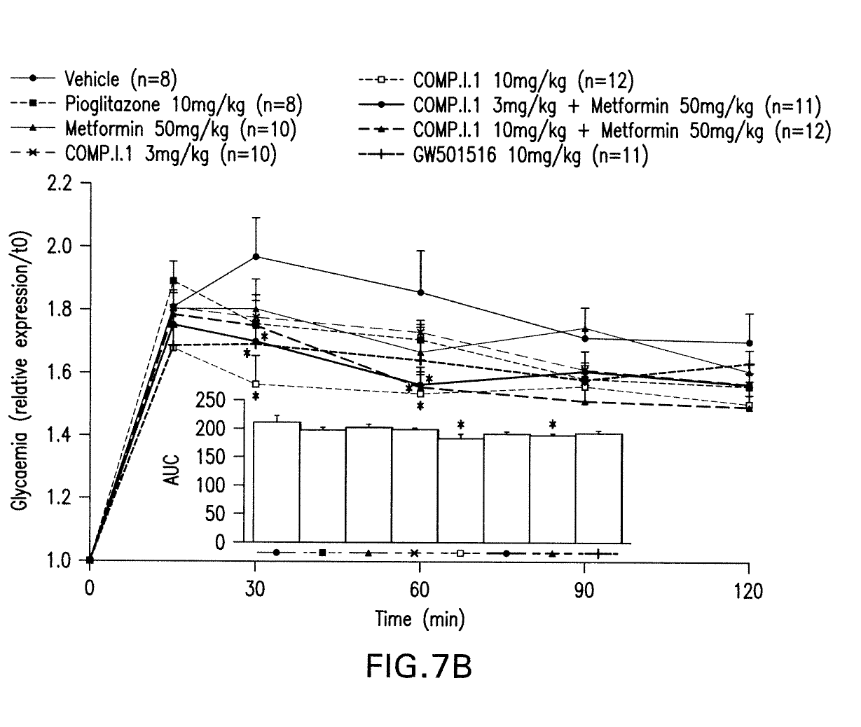

After 6 hours of fasting, rat received 2.5 g/kg glucose by the oral route. Glycaemia reached 186±5.4 mg/dl and 201±9 mg/dl at 15 and 30 minutes respectively in the vehicle group (FIG. 7A). This correspond to an 81 and 100% increase compared to the 0 (FIG. 7B). Pioglitazone and GW501516 had a strong tendency to improved glucose tolerance in a general manner. Even if the AUC was not significantly decreased, the variation of glycemia was significantly different from the vehicle group at 30 minutes (FIGS. 7A, 7B). Metformin and the compound I.1 in a dose of 3 mg/kg had a slight tendency to improve glucose tolerance but it was not statistically significant. On the other hand, the AUC showed that the compound I.1 in a dose of 10 mg/kg significantly improved glucose tolerance. There was not a clear potentiating effect when the rats were treated with the combination of the two compounds, whatever the dose used (FIGS. 7A, 7B).

Insulin Resistance Assessment after 5 Weeks of Treatment

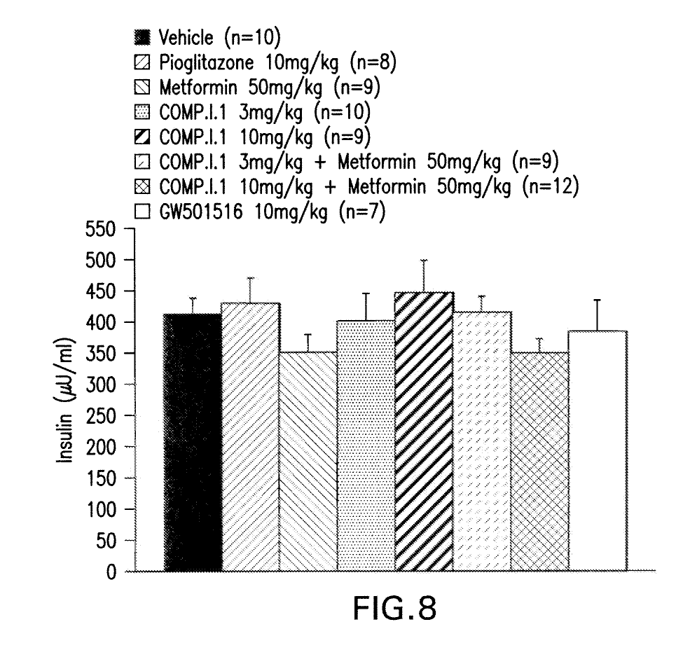

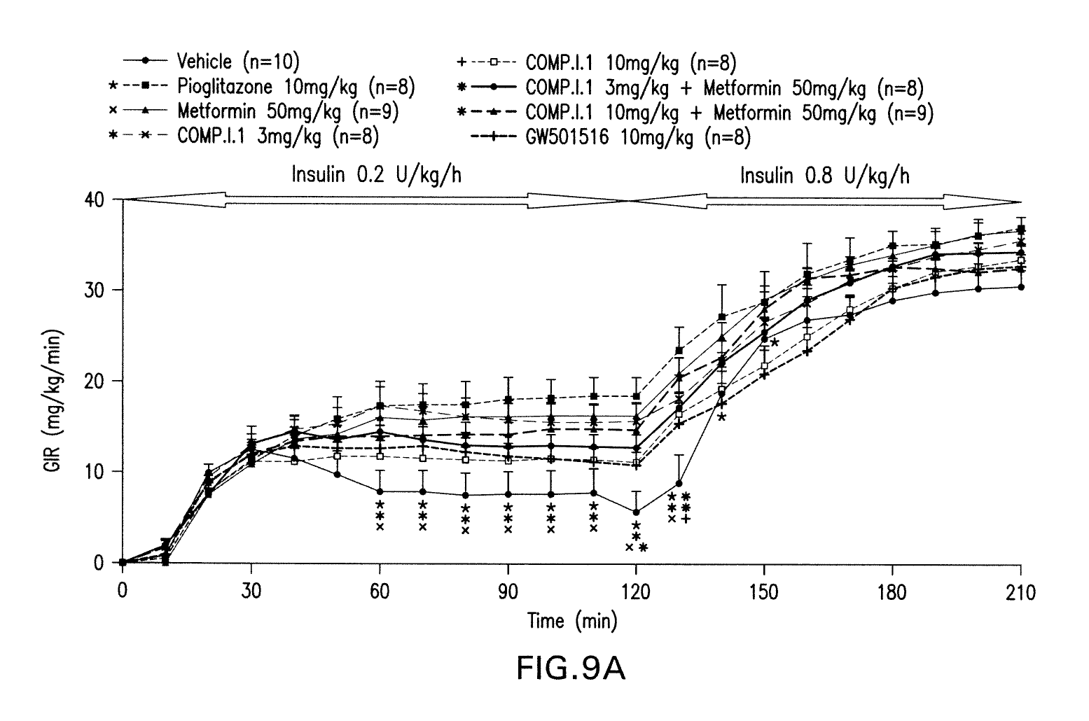

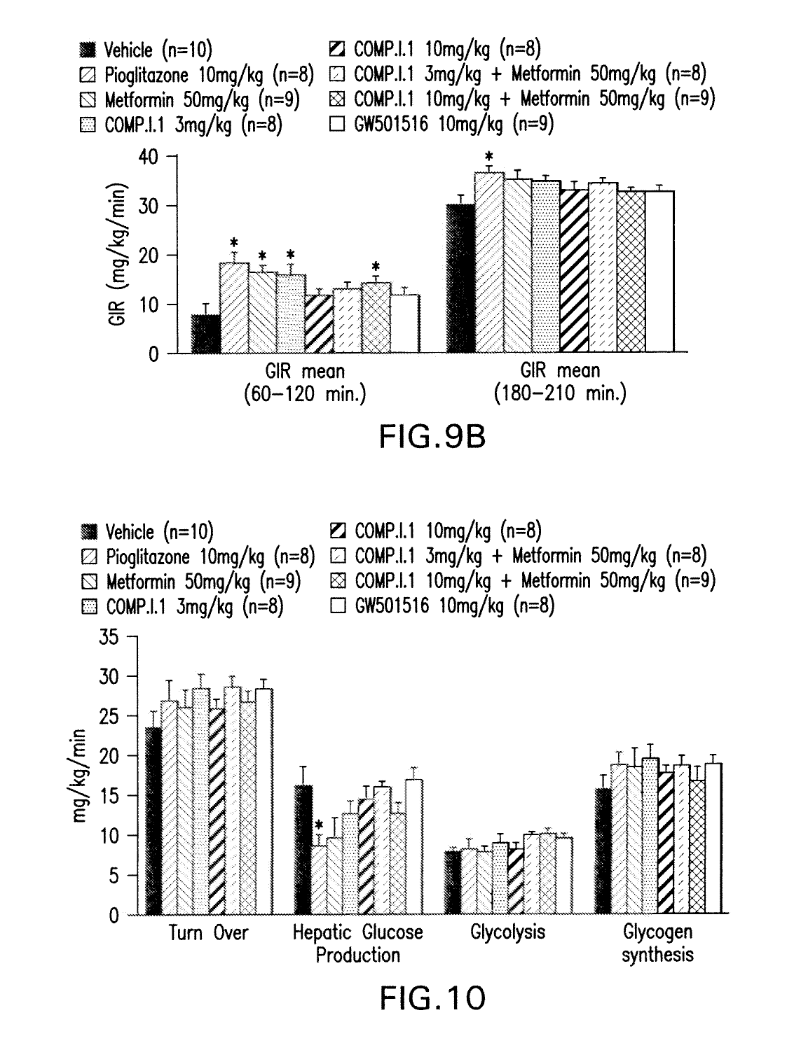

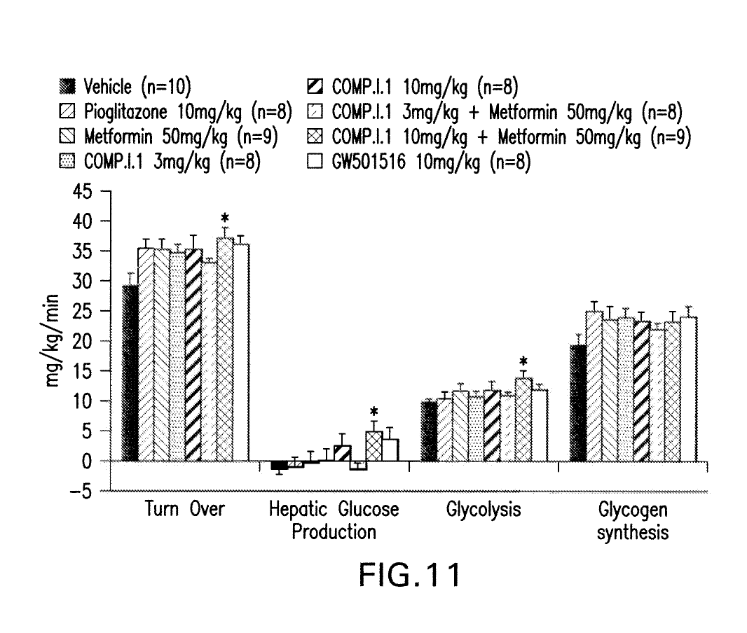

Clamp procedure was performed after 5 weeks of treatment, in 6 h fasting conditions. 0.2 U/kg/h of insulin was infused the first 2 hours. This physiological dose did not totally inhibit HGP, so an effect on this last one was assessed. 0.8 U/kg/h of insulin was infused thereafter. This pharmacological dose totally inhibits HGP, so the GTO rate corresponded to the glucose utilization rate specifically in peripheral tissues (such as muscles and adipose tissues). Plasma insulin level was measured to ensure that all groups were infused with similar doses of insulin (from 445 to 350 μU/ml, FIG. 8). FIG. 9A shows the GIR evolution during the infusion. GIR of the vehicle group was lower than all others groups during the first steady state (˜60-120 min). Pioglitazone, metformin, the compound I.1 in a dose of 3 mg/kg and the compound I.1 in a dose of 10 mg/kg+metformin increased GIR in a significant manner. At 0.8 U/kg/h, pioglitazone and metformin both tended to increase GIR (not significantly). The other compounds were less effective. When GIR was expressed as mean during the steady state GIR of the vehicle group was 7.7±2.4 mg/kg/min during the first step and 30±1.9 mg/kg/min during the second step (FIG. 9B). GIR during the first step was significantly increased by pioglitazone (138%), metformin (112%), the compound I.1 in a dose of 3 mg/kg (105%), and the compound I.1 in a dose of 10 mg/kg+metformin (82%) treatments. The other treatments increased GIR (from 50 to 70%) but not in a significant manner. Only pioglitazone significantly improved GIR during the second step. Under 0.2 U/kg/h, GTO in the vehicle group was 23.5±2 mg/kg/min, HGP was 16.1±2.4 mg/kg/min, glycolysis was 7.8±0.6 mg/kg/min, and glycogen synthesis was 15.6±1.7 mg/kg/min (FIG. 10). All treatments had a tendency to increase GTO (10-22%) and glycogen synthesis rate (18-25%, except the compound I.1 in a dose of 10 mg/kg alone or associated with metformin). Only the compound I.1 associated with metformin and GW501516 had a tendency to increased glycolysis (22-27%). HGP was significantly decreased by pioglitazone (47%) and not significantly by metformin (40%), the compound I.1 in a dose of 3 mg/kg, and the compound I.1 in a dose of 10 mg/kg+metformin (22% for both). Under 0.8 U/kg/h, GTO in the vehicle group was 29.2±2.1 mg/kg/min, HGP was −1.1±1.1 mg/kg/min, glycolysis was 9.8±0.6 mg/kg/min, and glycogen synthesis was 19.4±1.7 mg/kg/min (FIG. 11). All treatments had again a tendency to increase GTO (19-27%) and glycogen synthesis (13-29%). Only the association of the compound I.1 in a dose of 10 mg/kg with metformin had a significant effect on GTO (p<0.05). Metformin, the compound I.1 in a dose of 10 mg/kg (alone or in association with metformin), and GW501516 increased glycolysis (20, 42 and 21% respectively). Taken together, these results show that pioglitazone and metformin have insulin sensitizing effects at both the liver (observation made under physiological dose of insulin), and peripheral tissue levels. Pioglitazone effect was in favour to an increased storage into glycogen whereas metformin had a tendency to increase both glucose oxidation and storage. The compound I.1 had an insulin sensitizing effect, less marked than metformin, and not in a dose dependant manner. The association with metformin seems to be relevant since it increased whole body glucose oxidation rate. The compound I.1 seemed to better improve liver insulin resistance compared to GW501516. They both slightly increased glucose oxidation and storage.

Liver and Adipose Tissue Weights after 5 Weeks of Treatment

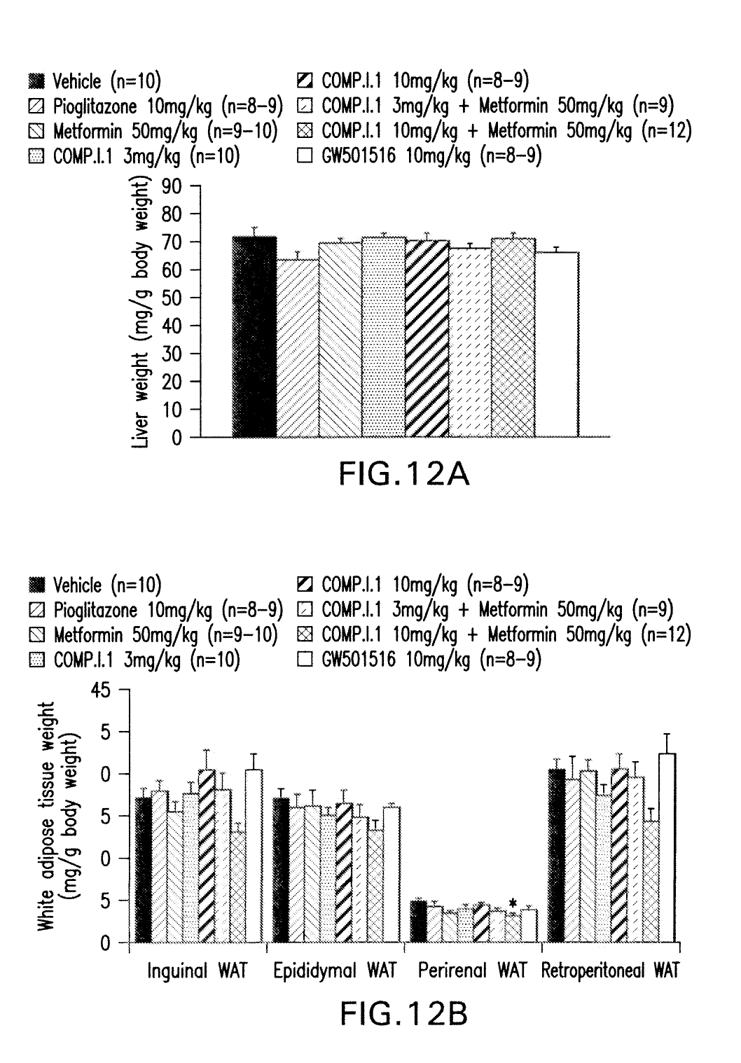

Tissues were weight after the clamp procedure. This animal model was characterized by a strong hepatic steatosis. Liver weight of the vehicle group was 38±1.4 g vs ˜15 g in normal rats (internal data). Pioglitazone and GW501516 both decreased liver weight even in a non significant manner (12 and 8% respectively, FIG. 12A). Most of the treatments did not modify subcutaneous or deep retroperitoneal and epididymal white adipose tissues. Only the combination of the compound I.1 in a dose of 10 mg/kg and metformin decreased the weight of these tissues (24%, 30% and 22% respectively). Metformin alone or in association with the compound I.1 in a dose of 3 and 10 mg/kg had a tendency to decrease perirenal white adipose tissue (by 27, 23 and 34%) (FIG. 12B).

Triglycerides Content in the Liver after 5 Weeks of Treatment

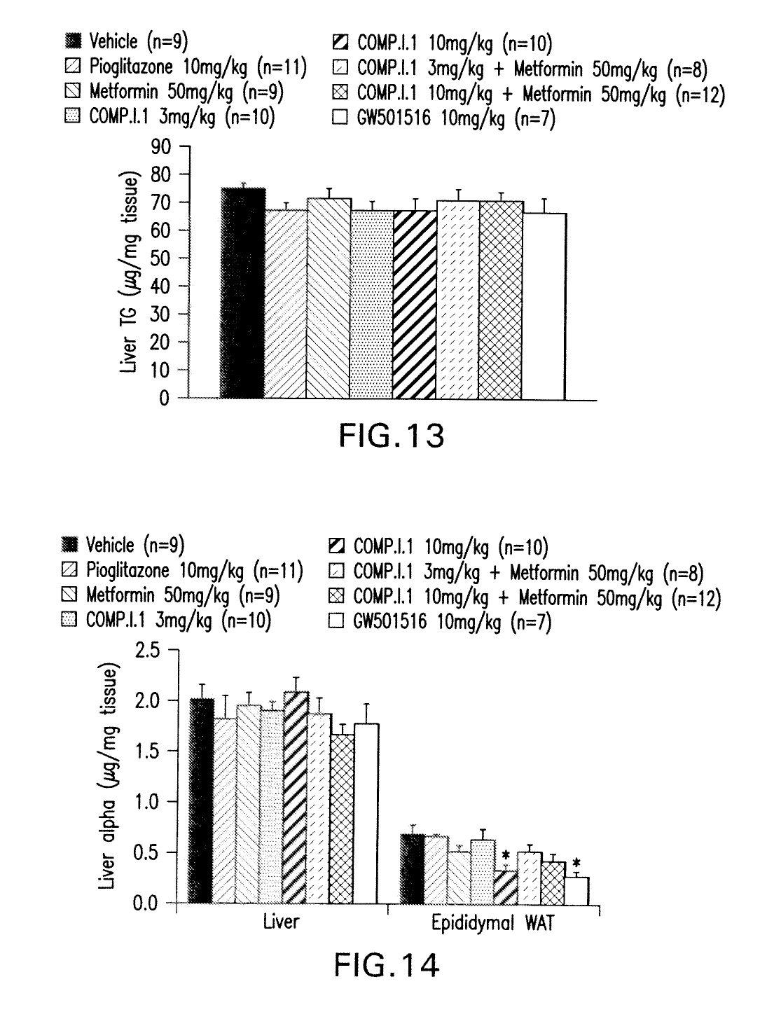

Pioglitazone, the compound I.1 (at any used dose) and GW501516 did not significantly decrease triglycerides content in the liver (FIG. 13).

TNF-α Levels in Liver and Epididymal Adipose Tissue after 5 Weeks of Treatment.

FIG. 14 shows that TNF-α level was unchanged in the liver in most of the treated rats. However metformin+the compound I.1 in a dose of 10 mg/kg and GW501516 tended to decrease TNF-α level (17% and 12% respectively). On the other hand, metformin, alone or associated with the compound I.1 in a dose of 3 and 10 mg/kg tended to decrease TNF-α levels in epididymal white adipose tissues (25, 25, and 38% respectively), the compound I.1 in a dose of 10 mg/kg and GW501516 significantly decreased it (51 and 60% respectively).

Histological Analyses for Succinate Dehydrogenase

The compound I.1 increased by about 50% the number of oxidative fiber in tibialis muscle (FIG. 1).

This study showed that the compound I.1 improved glucose homeostasis in rats fed with a high fat/medium fructose diet in a lesser extent than pioglitazone or metformin but in a better extent than GW501516. The effects observed were not clearly dose-dependent. The compound I.1 improved glucose tolerance, and insulin resistance (decreased HOMA-IR, increased GIR). The combination with metformin seemed to better improve HOMA-IR, reduce body weight gain, with a decreasing effect on adipose tissue mass and an increased glucose oxidation rate. This last result was reinforced by the increased number of oxidative fibers in tibialis muscle in rats treated with the compound I.1.

All publications and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

We claim:

1. A method for treating diseases of the lung selected form the group consisting of chronic obstructive airways disease (COAD), chronic obstructive pulmonary disease (COPD), adult onset asthma, emphysema or juvenile onset asthma, comprising administering a compound of formula (I) or a salt thereof: 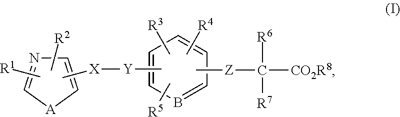
wherein:
R¹ is phenyl, naphthyl, pyridyl, thienyl, furyl, quinolyl or benzothienyl, any of which can have substituents selected from the group consisting of C₁₋₈ alkyl, C₁₋₈ alkyl having halogen, C₁₋₈ alkoxy, C₁₋₈ alkoxy having halogen, C₂₋₈ alkenyl, C₂₋₈ alkynyl, halogen, C₂₋₇ acyl, benzoyl, hydroxyl, nitro, amino, phenyl and pyridyl;
R² is C₁₋₈ alkyl, C₁₋₈ alkyl having halogen, C₂₋₈ alkenyl, C₂₋₈ alkynyl, 3-7 membered cycloalkyl, C₁₋₈ alkyl having 3-7 membered cycloalkyl, or C₁₋₆ alkyl substituted with phenyl, naphthyl or pyridyl, any of which can have substituents selected from the group consisting of C₁₋₈ alkyl, C₁₋₈ alkyl having halogen, C₁₋₈ alkoxy, C₁₋₈ alkoxy having halogen, C₂₋₈ alkenyl, C₂₋₈ alkynyl, halogen, C₂₋₇ acyl, benzoyl, hydroxyl, nitro, amino, phenyl and pyridyl;
A is oxygen, sulfur or NR⁹ in which R⁹ is hydrogen or C₁₋₈ alkyl;
X is a C₁₋₈ alkylene chain which can have substituents selected from the group consisting of C₁₋₈ alkyl, C₁₋₈ alkoxy and hydroxyl and which can contain a double bond;
Y is C(═=O), C(═=N—-OR¹⁰), CH(OR¹¹), CH═=CH, C—-C, or C(═=CH₂) in which each of R¹⁰ and R¹¹ is hydrogen or C₁₋₈ alkyl;
each of R³, R⁴ and R⁵ is hydrogen, C₁₋₈ alkyl, C₁₋₈ alkyl having halogen, C₁₋₈ alkoxy, C₁₋₈ alkoxy having halogen, C₂₋₈ alkenyl, C₂₋₈ alkynyl, halogen, C₂₋₇ acyl, benzoyl, hydroxyl, nitro, amino, phenyl, or pyridyl;
B is CH or nitrogen;
Z is oxygen or sulfur;
each of R⁶ and R⁷ is hydrogen, C₁₋₈ alkyl, C₁₋₈ alkyl having halogen;
R⁸ is hydrogen or C₁₋₈ alkyl;
provided that at least one of R³, R⁴ and R⁵ is not hydrogen;
or formula (II) or a salt thereof: 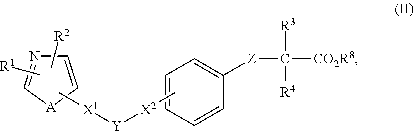
wherein:
each of R¹ and R² independently is a hydrogen atom, a halogen atom, nitro, an alkyl group having 1-8 carbon atoms, an alkoxy group having 1-8 carbon atoms, an alkyl group having 1-8 carbon atoms which has 1 to 3 halogen substituents, an alkoxy group having 1-8 carbon atoms which has 1 to 3 halogen substituents, an alkenyl group having 2-8 carbon atoms, an alkynyl group having 2-8 carbon atoms, a 3-7 membered cycloalkyl group, an alkyl group having 1-8 carbon atom which has a 3-7 membered cycloalkyl substituent, an aryl group having 6-10 carbon atoms which optionally has a substituent, an arylalkyl group which has a C₆₋₁₀ aryl portion and C₁₋₈ alkyl portion, a heterocyclic group which optionally has a substituent or a heterocyclic-alkyl group having an alkyl group of 1-8 carbon atoms;
A is an oxygen atom, a sulfur atom, or NR³ in which R³ is a hydrogen atom or an alkyl group having 1-8 carbon atoms;
each of X and Z independently is —C(═O)—, —C(═O)NH—, —C(═N—OR⁴)—, —CH(OR⁵)—, —NH(C═O)—, —NHSO₂—, —SO₂NH—, —CH═CH—, —C≡C—, or a bond in which each of R⁴ and R⁵ is a hydrogen atom or an alkyl group having 1-8 carbon atoms;
Y is an alkylene chain having 1-8 carbon atoms;
or formula (III) or a salt thereof: 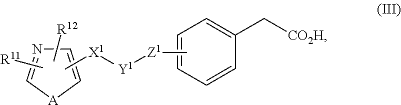
wherein:
each of R¹¹ and R¹² independently is a hydrogen atom, a halogen atom, nitro, hydroxyl, amino, an alkyl group having 1-8 carbon atoms, an alkoxy group having 1-8 carbon atoms, an alkyl group having 1-8 carbon atoms which has 1 to 3 halogen substituents, an alkoxy group having 1-8 carbon atoms which has 1 to 3 halogen substituents, an alkenyl group having 2-8 carbon atoms, an alkynyl group having 2-8 carbon atoms, a 3-7 membered cycloalkyl group, an alkyl group having 1-8 carbon atoms which has a 3-7 membered cycloalkyl substituent, or a phenyl, naphthyl, benzyl, phenethyl, pyridyl, thienyl, furyl, quinolyl, or benzothienyl group which optionally has a substituent selected from the group consisting of a halogen atom, nitro, hydroxyl, amino, an alkyl group having 1-8 carbon atoms, an alkoxy group having 1-8 carbon atoms, an alkyl group having 1-8 carbon atoms which has 1 to 3 halogen substituents, an alkoxy group having 1-8 carbon atoms which has 1 to 3 halogen substituents, an alkenyl group having 2-8 carbon atoms, an alkynyl group having 2-8 carbon atoms, a 3-7 membered cycloalkyl group, an alkyl group having 1-8 carbon atoms which has a 3-7 membered cycloalkyl substituent, phenyl and pyridyl;
each of X¹ and Z¹ independently is —C(═O)—, —C(═O)NH—, —C(═N—OR¹⁴)—, —CH(OR¹⁵)—, —NH(C═O)—, —NHSO₂—, —SO₂NH—, —CH═CH—, —C≡C—, or a bond in which each of R¹⁴ and R¹⁵ is a hydrogen atom or an alkyl group having 1-8 carbon atoms;
Y¹ is an alkylene chain having 1-8 carbon atoms;
or formula (IV) or a salt thereof: 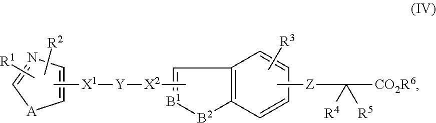
wherein:
A is O, S or NR⁷ in which R⁷ is hydrogen or C₁₋₈ alkyl;
B¹ is CW or N in which W is hydrogen or a bond; B² is O, S or NR⁸ in which R⁸ is hydrogen or C₁₋₈ alkyl;
each of X¹ and X² is O, S, NH, NHC(═O), C(═O), C(═N—OR⁹, CH(OR¹⁰), C═C, C≡C or a bond in which each of R⁹ and R¹⁰ is hydrogen or C₁₋₈ alkyl;
Y is a C₁₋₈ alkylene chain, which can be substituted with C₁₋₈ alkyl or C₁₋₈ alkyl substituted with 1-3 halogens;
Z is NH, O or S;
R¹ is aryl, which can be substituted with a group or atom selected from the group consisting of C₁₋₈ alkyl, C₁₋₈ alkoxy, C₁₋₈ alkyl substituted with 1-3 halogens, hydroxyl, nitro, amino, phenyl, pyridyl and halogen, or a heterocyclic group having five to eight membered ring comprising one to three hetero atoms selected from the group consisting of nitrogen, oxygen and sulfur and the other atoms consisting of carbon (benzene ring can be condensed with the heterocyclic ring);
R² is C₂₋₈ alkyl, C₁₋₈ alkyl substituted with 1-3 halogens, C₃₋₇ cycloalkyl, C₂₋₈ alkenyl, C₂₋₈ alkynyl, alkyl (comprising C₁₋₄ alkyl moiety) substituted with aryl, which can be substituted with a group or atom selected from the group consisting of C₁₋₈ alkyl, C₁₋₈ alkoxy, C₁₋₈ alkyl substituted with 1-3 halogens, hydroxyl, nitro, amino, phenyl, pyridyl and halogen, or alkyl (comprising C₁₋₄ alkyl moiety) substituted with a heterocyclic group having five to eight membered ring (comprising one to three hetero atoms selected from the group consisting of nitrogen, oxygen and sulfur and the other atoms consisting of carbon);
R³ is halogen, trifluoromethyl, C₁₋₈ alkyl, C₂₋₈ alkenyl or C₂₋₈ alkynyl;
each of R⁴ and R⁵ is hydrogen, C₁₋₈ alkyl or C₁₋₈ alkyl substituted with 1-3 halogens; and R⁶ is hydrogen, C₁₋₈ alkyl substituted with amino, C₁₋₈ alkyl or alkali metal;
provided that each of Z and R³ is attached to the benzene ring, and X² is not attached to the benzene ring;
or formula (V) or salt thereof: 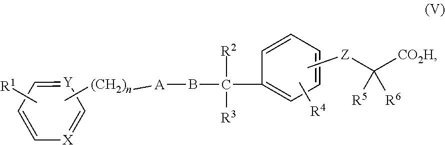
wherein:
R¹ and R⁴ are the same or different and each represents a hydrogen atom, an alkyl group having 1 to 8 carbon atoms, an alkenyl group having 2 to 8 carbon atoms, an alkynyl group having 2 to 8 carbon atoms, an alkoxy group having 1 to 8 carbon atoms, a halogen atom, an alkyl group having 1 to 8 carbon atoms and a halogen atom substituent, an alkoxy group having 1 to 8 carbon atoms and a halogen atom substituent, a hydroxyl group, a nitro group, an acyl group having 2 to 8 carbon atoms, an aryl group having 6 to 10 carbon atoms, or a 5- or 6-membered heterocyclic group;
R² represents a hydrogen atom;
R³ represents an alkyl group having 1 to 8 carbon atoms, or R³ is combined with R² to represent ═O or ═C(R⁷)(R⁸) in which R⁷ and R⁸ are the same or different and each represents a hydrogen atom or an alkyl group having 1 to 8 carbon atoms;
R⁵ and R⁶ are the same or different and each represents a hydrogen atom, an alkyl group having 1 to 8 carbon atoms, or an alkyl group having 1 to 8 carbon atoms and a halogen atom substituent;
X and Y are the same or different and each represents CH or N;
Z represents an oxygen atom or a sulfur atom;
A represents a 5-membered heterocyclic group selected from the group consisting of pyrazole, thiophene, furan and pyrrole which optionally has an alkyl substituent having 1 to 8 carbon atoms which has a substituent selected from the group consisting of an alkyl group having 1 to 8 carbon atoms, a 3- to 7-membered cycloalkyl group, an alkenyl group having 2 to 8 carbon atoms, an alkynyl group having 2 to 8 carbon atoms, an alkoxy group having 1 to 8 carbon atoms, an alkyl group which has 1 to 8 carbon atoms and a 3- to 7-membered cycloalkyl group substituent, an alkyl group having 1 to 8 carbon atoms and a halogen atom substituent, an alkoxy group having 1 to 8 carbon atoms and a halogen atom substituent, an aryl group having 6 to 10 carbon atoms, a 5- or 6-membered heterocyclic group, an aralkyl group having an aryl moiety of 6 to 10 carbon atoms and an alkylene moiety of 1 to 8 carbon atoms, and 5- or 6-membered heterocyclic group;
B represents an alkylene chain having 1 to 8 carbon atoms which optionally has a substituent selected from the group consisting of an alkyl group having 1 to 8 carbon atoms, a 3- to 7-membered cycloalkyl group, an alkenyl group having 2 to 8 carbon atoms, an alkynyl group having 2 to 8 carbon atoms, an alkoxy group having 1 to 8 carbon atoms, a halogen atom, an alkyl group having 1 to 8 carbon atoms and a halogen atom substituent, and an alkoxy group having 1 to 8 carbon atoms and a halogen atom substituent, the alkylene group optionally having a double bond in the case that the alkylene group has 2 to 6 carbon atoms; and
n is an integer of 0 to 5;
or formula (VI) or salts thereof: 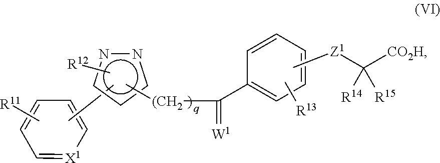
wherein:
R¹¹ and R¹³ are the same or different and each represents a hydrogen atom, an alkyl group having 1 to 8 carbon atoms, an alkenyl group having 2 to 8 carbon atoms, an alkynyl group having 2 to 8 carbon atoms, an alkoxy group having 1 to 8 carbon atoms, a halogen atom, an alkyl group having 1 to 8 carbon atoms and a halogen atom substituent, an alkoxy group having 1 to 8 carbon atoms and a halogen atom substituent, a hydroxyl group, a nitro group, an acyl group having 2 to 8 carbon atoms, an aryl group having 6 to 10 carbon atoms, or a 5- or 6-membered heterocyclic group;
R¹² represents a hydrogen atom, an alkyl group having 1 to 8 carbon atoms, a 3- to 7-membered cycloalkyl group, an alkenyl group having 2 to 8 carbon atoms, an alkynyl group having 2 to 8 carbon atoms, an alkoxy group having 1 to 8 carbon atoms, an alkyl group having 1 to 8 carbon atoms and a 3- to 7-membered cycloalkyl group substituent, an alkyl group having 1 to 8 carbon atoms and a halogen atom substituent, an alkoxy group having 1 to 8 carbon atoms and a halogen atom substituent, an aryl group having 6 to 10 carbon atoms, a 5- or 6-membered heterocyclic group, an aralkyl group having an aryl moiety of 6 to 10 carbon atoms and an alkylene moiety of 1 to 8 carbon atoms, or an alkyl group having 1 to 8 carbon atoms and a 5- or 6-membered heterocyclic substituent;
R¹⁴ and R¹⁵ are the same or different and each represents a hydrogen atom, an alkyl group having 1 to 8 carbon atoms, or an alkyl group having 1 to 8 carbon atoms and a halogen atom substituent;
X¹ represents CH or N;
Z¹ represents an oxygen atom or a sulfur atom;
W¹ represents an oxygen atom or CH₂; and
q is an integer of 2 to 4;
or formula (VII) or salts thereof: 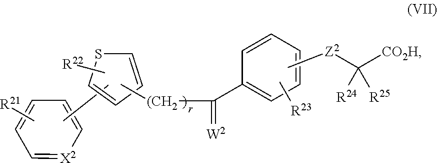
wherein:
R²¹ and R²³ are the same or different and each represents a hydrogen atom, an alkyl group having 1 to 8 carbon atoms, an alkenyl group having 2 to 8 carbon atoms, an alkynyl group having 2 to 8 carbon atoms, an alkoxy group having 1 to 8 carbon atoms, a halogen atom, an alkyl group having 1 to 8 carbon atoms and a halogen atom substituent, an alkoxy group having 1 to 8 carbon atoms and a halogen atom substituent, a hydroxyl group, a nitro group, an acyl group having 2 to 8 carbon atoms, an aryl group having 6 to 10 carbon atoms, or a 5- or 6-membered heterocyclic group;
R²² represents a hydrogen atom, an alkyl group having 1 to 8 carbon atoms, a 3- to 7-membered cycloalkyl group, an alkenyl group having 2 to 8 carbon atoms, an alkynyl group having 2 to 8 carbon atoms, an alkoxy group having 1 to 8 carbon atoms, an alkyl group having 1 to 8 carbon atoms and a 3- to 7-membered cycloalkyl group substituent, an alkyl group having 1 to 8 carbon atoms and a halogen atom substituent, an alkoxy group having 1 to 8 carbon atoms and a halogen atom substituent, an aryl group having 6 to 10 carbon atoms, a 5- or 6-membered heterocyclic group, an aralkyl group having an aryl moiety of 6 to 10 carbon atoms and an alkylene moiety of 1 to 8 carbon atoms, or an alkyl group having 1 to 8 carbon atoms and a 5- or 6-membered heterocyclic substituent;
R²⁴ and R²⁵ are the same or different and each represents a hydrogen atom, an alkyl group having 1 to 8 carbon atoms, or an alkyl group having 1 to 8 carbon atoms and a halogen atom substituent;
X² represents CH or N;
Z² represents an oxygen atom or a sulfur atom;
W² represents an oxygen atom or CH); and
r is an integer of 2 to 4;
or formula (VIII) or salts thereof: 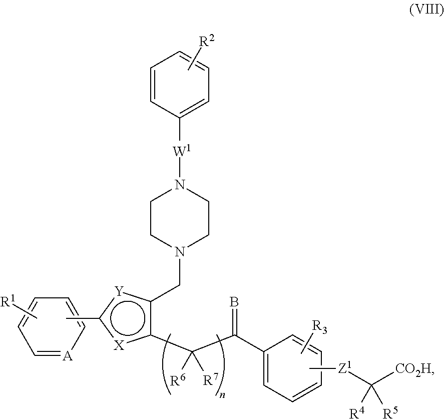
wherein:
A represents CH or a nitrogen atom;
B represents an oxygen atom or C(R⁸)(R⁹) in which each of R⁸ and R⁹ independently represents a hydrogen atom or an alkyl group having 1 to 8 carbon atoms;
W¹ represents a bond, C(═O), or (—C(R¹⁰)(R¹¹)—)ₘ in which each of R¹⁰ and R¹¹ independently a hydrogen or an alkyl group having 1 to 8 carbon atoms and m is an integer of 1 to 3;
X and Y differ from each other, and each represents an oxygen atom, a sulfur atom, a nitrogen atom, or CR¹² in which R¹² represents a hydrogen atom or an alkyl group having 1 to 8 carbon atoms;
Z¹ represents a bond, an oxygen atom, a sulfur atom, or C(R¹³)(R¹⁴) in which each of R¹³ and R¹⁴ independently represents a hydrogen atom or an alkyl group having 1 to 8 carbon atoms;
each of R¹, R² and R³ independently represents a hydrogen atom, an alkyl group having 1 to 8 carbon atoms, an alkenyl group having 2 to 8 carbon atoms, an alkynyl group having 2 to 8 carbon atoms, an alkoxy group having 1 to 8 carbon atoms, a halogen atom, an alkyl group having 1 to 8 carbon atom which is substituted with a halogen atom, an alkoxy group having 1 to 8 carbon atom which is substituted with a halogen atom, hydroxyl, nitro, an acyl group having 2 to 8 carbon atoms, an aryl group having 6 to 10 carbon atoms, or a 5- or 6-membered heterocyclic group;
each of R⁴ and R⁵ independently represents a hydrogen atom, an alkyl group having 1 to 8 carbon atoms, or an alkyl group having 1 to 8 carbon atoms which is substituted with a halogen atom;
each of R⁶ and R⁷ independently represents a hydrogen atom, an alkyl group having 1 to 8 carbon atoms, an alkenyl group having 2 to 8 carbon atoms, an alkynyl group having 2 to 8 carbon atoms, or an alkyl group having 1 to 8 carbon atoms which is substituted with a halogen atom; and
n represents an integer of 1 to 5;
or formula (IX) or salts thereof: 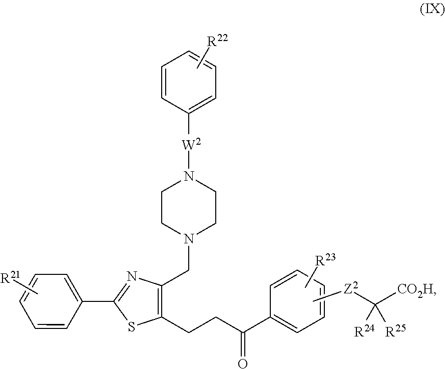
wherein:
W² represents a bond, C(═O), or —CH₂;
Z² represents an oxygen atom or a sulfur atom;
each of R²¹, R²² and R²³ independently represents a hydrogen atom, an alkyl group having 1 to 8 carbon atoms, an alkenyl group having 2 to 8 carbon atoms, an alkynyl group having 2 to 8 carbon atoms, an alkoxy group having 1 to 8 carbon atoms, a halogen atom, an alkyl group having 1 to 8 carbon atom which is substituted with a halogen atom, an alkoxy group having 1 to 8 carbon atom which is substituted with a halogen atom, hydroxyl, nitro, an acyl group having 2 to 8 carbon atoms, an aryl group having 6 to 10 carbon atoms, or a 5- or 6-membered heterocyclic group;
each of R²⁴ and R²⁵ independently represents a hydrogen atom, an alkyl group having 1 to 8 carbon atoms, or an alkyl group having 1 to 8 carbon atoms which is substituted with a halogen atom;
or formula (X) or salts thereof: 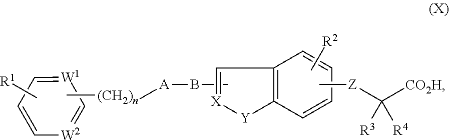
wherein:
each of W¹ and W² independently represents a nitrogen atom or CH′
X represents a nitrogen atom or CH;
Y represents an oxygen atom or a sulfur atom;
Z represents a bond, an oxygen atom, a sulfur atom or NR⁵, in which R⁵ represents a hydrogen atom or an alkyl group having 1 to 8 carbon atoms;
each of R¹ and R² independently represents a hydrogen atom, a halogen atom, a hydroxyl group, a nitro group, an amino group, an alkyl group having 1 to 8 carbon atoms, a 3- to 7-membered cycloalkyl group, an alkenyl group having 2 to 8 carbon atoms, an alkynyl group having 2 to 8 carbon atoms, an alkoxy group having 1 to 8 carbon atoms, an alkyl group having 1 to 8 carbon atoms and a 3- to 7-membered cycloalkyl substituent, an alkyl group having 1 to 8 carbon atoms and a halogen substituent, an alkoxy group having 1 to 8 carbon atoms and a halogen substituent, an aryl group having 6 to 10 carbon atoms, 5- or 6-membered heterocyclic group, an aralkyl group having an aryl moiety of 6 to 10 carbon atoms and an alkylene moiety of 1 to 8 carbon atoms, or an alkyl group having 1 to 8 carbon atoms and a 5- or 6-membered heterocyclic substituent;
each of R³ and R⁴ independently represents a hydrogen atom, an alkyl group having 1 to 8 carbon atoms, or an alkyl group having 1 to 8 carbon atoms and a halogen substituent;
A represents a 5-membered hetero ring selected from the group consisting of pyrazole, thiophene, furan, isoxazole, isothiazole and pyrrole, in which the 5-membered hetero ring may have a substituent selected from the group consisting of a halogen atom, a hydroxyl group, a nitro group, an amino group, an alkyl group having 1 to 8 carbon atoms, a 3- to 7-membered cycloalkyl group, an alkenyl group having 2 to 8 carbon atoms, an alkynyl group having 2 to 8 carbon atoms, an alkoxy group having 1 to 8 carbon atoms, an alkyl group having 1 to 8 carbon atoms and a 3- to 7-membered cycloalkyl substituent, an alkyl group having 1 to 8 carbon atoms and a halogen substituent, an alkoxy group having 1 to 8 carbon atoms and a halogen substituent, an aryl group having 6 to 10 carbon atoms, a 5- or 6-membered heterocyclic group, an aralkyl group having an aryl moiety of 6 to 10 carbon atoms and an alkylene moiety of 1 to 8 carbon atoms, and an alkyl group having 1 to 8 carbon atoms and a 5- or 6-membered heterocyclic substituent;
B represents a bond or an alkylene chain having 1 to 8 carbon atoms which may have a substituent selected from the group consisting of an alkyl group having 1 to 8 carbon atoms, a 3- to 7-membered cycloalkyl group, an alkoxy group having 1 to 8 carbon atoms and a halogen substituent and further may have a double or triple bond; and
n is an integer of 0 to 3;
or formula (XI) or salts thereof: 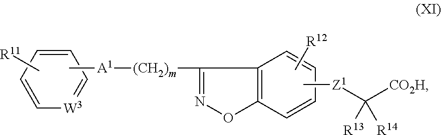
wherein:
W³ represents a nitrogen atom or CH;
Z¹ represents an oxygen atom or a sulfur atom;
each of R¹¹ and R¹² independently represents a hydrogen atom, a halogen atom, a hydroxyl group, a nitro group, an amino group, an alkyl group having 1 to 8 carbon atoms, an alkoxy group having 1 to 8 carbon atoms, an alkyl group having 1 to 8 carbon atoms and a halogen substituent, or an alkoxy group having 1 to 8 carbon atoms and a halogen substituent;
each of R¹³ and R¹⁴ independently represents a hydrogen atom or an alkyl group having 1 to 8 carbon atoms;
A¹ represents pyrazole or thiophene which may have a substituent selected from the group consisting of a halogen atom, a hydroxyl group, a nitro group, an amino group, an alkyl group having 1 to 8 carbon atoms, an alkoxy group having 1 to 8 carbon atoms, an alkyl group having 1 to 8 carbon atoms and a halogen substituent, or an alkoxy group having 1 to 8 carbon atoms and a halogen substituent; and
m is an integer of 2 to 4;
or formula (XII) or salts thereof: 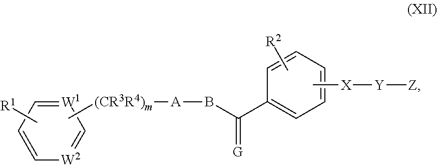
wherein:
each of W¹ and W² independently is CH or nitrogen;
X is NR⁵ or CR⁶R⁷, wherein R⁵ is hydrogen, C₁₋₈ alkyl, C₁₋₈ alkyl substituted with halogen, C₁₋₈ alkyl substituted with C₁₋₈ alkoxy, cycloalkyl of three-membered to seven-membered ring, C₁₋₈ alkyl substituted with cycloalkyl of three-membered to seven-membered ring, C₁₋₈ alkyl substituted with phenyl, C₂₋₈ acyl, or C₂₋₈ alkenyl, and each of R⁶ and R⁷ independently is hydrogen or C₁₋₈ alkyl;
Y is —(CR⁸R⁹)ₙ—, wherein each of R⁸ and R⁹ independently is hydrogen or C₁₋₈ alkyl, and n is 1 to 4; or
X and Y are combined to form —CR¹⁰═CR¹¹— or ethynylene, wherein each of R¹⁰ and R¹¹ independently is hydrogen or C₁₋₈ alkyl;
Z is carboxyl or tetrazolyl;
G is O, S or CR¹²R¹³, wherein each of R¹² and R¹³ independently is hydrogen or C₁₋₈ alkyl;
A is a five-membered heterocyclic ring selected from the group consisting of thiazole, oxazole, imidazole, pyrazole, thiophene, furan, and pyrrole, which can be substituted with a substituent selected from the group consisting of C₁₋₈ alkyl, C₂₋₈ alkenyl, C₂₋₈ alkynyl, C₁₋₈ alkoxy, halogen, C₁₋₈ alkyl substituted with halogen, C₁₋₈ alkoxy substituted with halogen, hydroxyl, nitro, C₂₋₈ acyl, C₆₋₁₀ aryl, and a five-membered or six-membered heterocyclic group;
B is a C₁₋₈ alkylene, C₂₋₈ alkenylene or C₂₋₈ alkynylene chain, wherein the chain can be substituted with a substituent selected from the group consisting of C₁₋₈ alkyl, cycloalkyl of three-membered to seven-membered ring, C₁₋₈ alkoxy, and halogen;
each of R¹ and R² independently is hydrogen, C₁₋₈ alkyl, C₂₋₈ alkenyl, C₂₋₈ alkynyl, C₁₋₈ alkoxy, halogen, C₁₋₈ alkyl substituted with halogen, C₁₋₈ alkoxy substituted with halogen, hydroxyl, nitro, C₂₋₈ acyl, C₆₋₁₀ aryl, or a five-membered or six-membered heterocyclic group;
each of R³ and R⁴ independently is hydrogen or C₁₋₈ alkyl; and
m is an integer of 0 to 3;
or formula (XIII) or salts thereof: 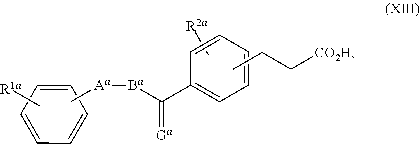
wherein:
wherein G^{a} is O, S or CH₂;
A^{a} is five-membered heterocyclic ring selected from the group consisting of thiazole, oxazole, and thiophene, which can be substituted with a substituent selected from the group consisting of C₁₋₈ alkyl, C₁₋₈ alkoxy, halogen, C₁₋₈ alkyl substituted with halogen, C₁₋₈ alkoxy substituted with halogen, hydroxyl, nitro, and C₂₋₈ acyl;
B^{a} is a C₁₋₈ alkylene or C₂₋₈ alkenylene chain; and
each of R^{1a} and R^{2a} independently is hydrogen, C₁₋₈ alkyl, C₁₋₈ alkoxy, halogen, C₁₋₈ alkyl substituted with halogen, C₁₋₈ alkoxy substituted with halogen, hydroxyl, nitro, or C₂₋₈ acyl;
or formula (XIV) or salts thereof: 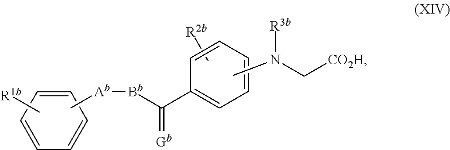
wherein:
G is O, S or CH₂;
A is a five-membered heterocyclic ring selected from the group consisting of thiazole, oxazole, and thiophene, which can be substituted with a substituent selected from the group consisting of C₁₋₈ alkyl, C₁₋₈ alkoxy, halogen, C₁₋₈ alkyl substituted with halogen, C₁₋₈ alkoxy substituted with halogen, hydroxyl, nitro, and C₂₋₈ acyl;
B^{b} is a C₁₋₈ alkylene or C₂₋₈ alkenylene chain;
each of R^{1b} and R^{2b} independently is hydrogen, C₁₋₈ alkyl, C₁₋₈ alkoxy, halogen, C₁₋₈ alkyl substituted with halogen, C₁₋₈ alkoxy substituted with halogen, hydroxyl, nitro, or C₂₋₈ acyl; and
R^{3b} is hydrogen or C₁₋₈ alkyl;
or formula (XV) or a salt thereof: 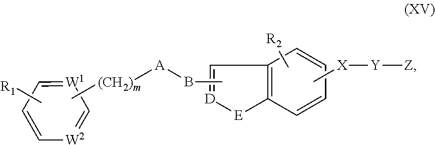
wherein:
each of W¹ and W² is independently CH or N;
X is NR³ or CR⁴R⁵, in which R³ is an alkyl group having 1 to 8 carbon atoms, an alkyl group having 1 to 8 carbon atoms and a halogen atom substituent, an alkyl group having 1 to 8 carbon atoms substituted with an alkoxy group having 1 to 8 carbon atoms, an alkyl group having 1 to 8 carbon atoms substituted with a 3-7 membered cycloalkyl group, an alkyl group having 1 to 8 carbon atoms substituted with a phenyl group, an acyl group having 2 to 8 carbon atoms or an alkenyl group having 2 to 8 carbon atoms;
each of R⁴ and R⁵ is independently H or an alkyl group having 1 to 8 carbon atoms;
Y is —(CR⁶R⁷)ₙ—, in which each of R⁶ and R⁷ is independently H or an alkyl group having 1 to 8 carbon atoms, and n is an integer of 1 to 4;
Z is a carboxylic group or a tetrazolyl group;
A is a 5 or 6-membered-heterocyclic group selected from the group consisting of thiazole, oxazole, imidazole, pyrazole, thiophene, furan, pyrrole, pyridine or pyrimidine, or a phenyl group, which may have a substituent selected from the group consisting of an alkyl group having 1 to 8 carbon atoms, a 3-7 membered cycloalkyl group, an alkenyl group having 2 to 8 carbon atoms, an alkynyl group having 2 to 8 carbon atoms, an alkoxyl group having 1 to 8 carbon atoms, an alkyl group having 1 to 8 carbon atoms substituted with a 3-7 membered cycloalkyl group, an alkyl group having 1 to 8 carbon atoms and a halogen atom substituent, an alkoxy group having 1 to 8 carbon atoms and a halogen atom substituent, an aryl group having 6 to 10 carbon atoms, a 5 or 6-membered heterocyclic group, aralkyl group comprising an aryl group having 6 to 10 carbon atoms and an alkyl group having 1 to 8 carbon atoms or an alkyl group having 1 to 8 carbon atoms substituted with a 5 or 6-membered heterocyclic group;
B is a bond or an alkylene chain having 1 to 8 carbon atoms which may have a substituent selected from the group consisting of an alkyl group having 1 to 8 carbon atoms, a 3-7 membered cycloalkyl group, an alkoxy group having 1 to 8 carbon atoms or a halogen atom and which may have a double bond or triple bond when the carbon number of alkylene chain is 2 or more;
D is N or CH;
E is O or S;
each of R¹ and R² is independently H, an alkyl group having 1 to 8 carbon atoms, an alkenyl group having 2 to 8 carbon atoms, an alkynyl group having 2 to 8 carbon atoms, an alkoxy group having 1 to 8 carbon atoms, a halogen atom, an alkyl group having 1 to 8 carbon atoms and a halogen atom substituent, an alkoxy group having 1 to 8 carbon atoms and a halogen atom substituent, hydroxyl, nitro, an acyl group having 2 to 8 carbon atoms, an aryl group having 6 to 10 carbon atoms or a 5 or 6-membered heterocyclic group; and
m is an integer of 0 to 3;
or formula (XVI) or a pharmaceutically acceptable salt thereof, 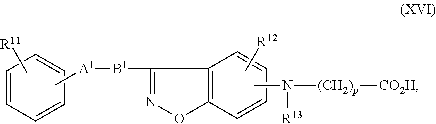
wherein:
R¹³ is an alkyl group having 1 to 8 carbon atoms or an alkyl group having 1 to 8 carbon atoms and a halogen atom substituent;
p is an integer of 1 to 4;
A¹ is thiazole, oxazole, pyridine, pyrimidine or phenyl which may have a substituent selected from the group consisting of an alkyl group having 1 to 8 carbon atoms or an alkyl group having 1 to 8 carbon atoms and a halogen atom substituent;
B¹ is an alkylene chain having 2 to 4 carbon atoms; and
each of R¹¹ and R¹² is independently H, an alkyl group having 1 to 8 carbon atoms, a halogen atom, an alkyl group having 1 to 8 carbon atoms and a halogen atom substituent,
wherein N(R¹³)((CH₂)ₚ—CO₂H) is attached to the 6th position of benzisoxazole;
or formula (XVII) or a salt thereof: 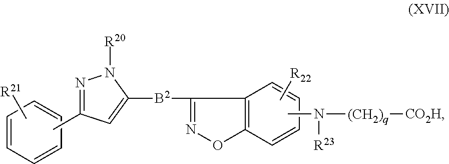
wherein:
R²³ is an alkyl group having 1 to 8 carbon atoms or an alkyl group having 1 to 8 carbon atoms and a halogen atom substituent;
q is an integer of 1 to 4;
R²⁰ is an alkyl group having 1 to 8 carbon atoms;
B² is an alkylene chain having 2 to 4 carbon atoms;
each of R²¹ and R²² is independently H, an alkyl group having 1 to 8 carbon atoms, a halogen atom, an alkyl group having 1 to 8 carbon atoms and a halogen atom substituent;
or formula (XVIII) or a salt thereof: 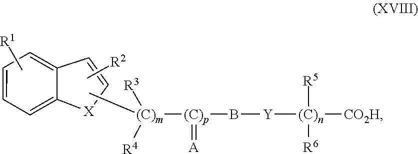
wherein:
R¹ represents hydrogen, halogen, hydroxyl, nitro, amino, cyano, carboxyl, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, a 5- or 6-membered heterocyclic group, an aralkyl group having an aryl moiety of 6-10 carbon atoms and an alkylene moiety of 1-8 carbon atoms, or an alkyl group having 1-8 carbon atoms and a 5- or 6-membered heterocyclic substituent;
R² represents hydrogen, an alkyl group having 1-8 carbon atoms, an alkenyl group having 2-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an acyl group having 2-8 carbon atoms, an aryl group having 6-10 carbon atoms, or an aralkyl group having an aryl moiety of 6-10 carbon atoms and an alkylene moiety of 1-8 carbon atoms;
each of R³, R⁴, R⁵ and R⁶ independently represents hydrogen, an alkyl group having 1-8 carbon atoms, or an alkyl group having 1-8 carbon atoms and having a halogen substituent;
X is oxygen, sulfur or NR⁷, R⁷ representing hydrogen, an alkyl group having 1-8 carbon atoms, an alkyl group having 1-8 carbon atoms and having a halogen substituent, an aralkyl group having an aryl moiety of 6-10 carbon atoms and an alkylene moiety of 1-8 carbon atoms, an acyl group having 2-8 carbon atoms, or an alkenyl group having 2-8 carbon atoms;
Y is oxygen, sulfur, NR⁸ or a bond, R⁸ representing hydrogen, an alkyl group having 1-8 carbon atoms, an alkyl group having 1-8 carbon atoms and having a halogen substituent, an acyl group having 2-8 carbon atoms, or an alkenyl group having 2-8 carbon atoms;
p is 0 or 1;
A is oxygen CH₂, N—NH₂ or N—OR⁹, R⁹ representing hydrogen, an alkyl group having 1-8 carbon atoms, an alkyl group having 1-8 carbon atoms and having a halogen substituent, an acyl group having 2-8 carbon atoms, an alkenyl group having 2-8 carbon atoms, or an aralkyl group having an aryl moiety of 6-10 carbon atoms and an alkylene moiety of 1-8 carbon atoms;
B represents, in the case of p=1, a benzene ring having or not having a substituent selected from the group consisting of halogen, hydroxyl, nitro, amino, an alkyl group having 1-8 carbon atoms, 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an alkoxy group having 1-8 carbon atoms and having a halogen substituent, an acyl group having 2-8 carbon atoms, an aryl group having 6-10 carbon atoms, or an aralkyl group having an aryl moiety of 6-10 carbon atoms and an alkylene moiety of 1-8 carbon atoms, and, in the case of p=0, a condensed ring selected from the group consisting of indole, benzofuran, benzisoxazole and 1,2-benzisothiazole, in which said condensed ring has or does not have a substituent selected from the group consisting of halogen, hydroxyl, nitro, amino, an alkyl group having 1-8 carbon atoms, 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an alkoxy group having 1-8 carbon atoms and having a halogen substituent, an acyl group having 2-8 carbon atoms, an aryl group having 6-10 carbon atoms, or an aralkyl group having an aryl moiety of 6-10 carbon atoms and an alkylene moiety of 1-8 carbon atoms;
Y is bonded to the benzene ring of B;
—(C(R³)(R⁴))ₘ— is bonded to the condensed ring of B at its 3-position;
m is an integer of 1 to 4;
n is an integer of 0 to 5; and
Y is a bond in the case of n=0;
or formula (XIX) or a pharmacologically acceptable salt thereof: 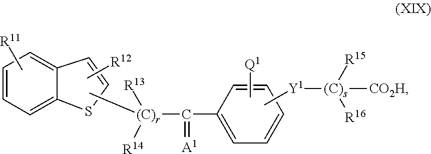
wherein:
R¹¹ represents hydrogen, halogen, hydroxyl, nitro, amino, cyano, carboxyl, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, a 5- or 6-membered heterocyclic group, an aralkyl group having an aryl moiety of 6-10 carbon atoms and an alkylene moiety of 1-8 carbon atoms, or an alkyl group having 1-8 carbon atoms and a 5- or 6-membered heterocyclic substituent,
R¹² represents hydrogen, an alkyl group having 1-8 carbon atoms, an alkenyl group having 2-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an acyl group having 2-8 carbon atoms, an aryl group having 6-10 carbon atoms, or an aralkyl group having an aryl moiety of 6-10 carbon atoms and an alkylene moiety of 1-8 carbon atoms,
each of R¹³, R¹⁴, R¹⁵ and R¹⁶ independently represents hydrogen, an alkyl group having 1-8 carbon atoms, or an alkyl group having 1-8 carbon atoms and having a halogen substituent,
Y¹ is oxygen, sulfur, NR¹⁸ or a bond. R¹⁸ representing hydrogen, an alkyl group having 1-8 carbon atoms, an alkyl group having 1-8 carbon atoms and having a halogen substituent, an acyl group having 2-8 carbon atoms, or an alkenyl group having 2-8 carbon atoms,
A¹ is oxygen CH₂, N—NH₂ or N—OR¹⁹, R¹⁹ representing hydrogen, an alkyl group having 1-8 carbon atoms, an alkyl group having 1-8 carbon atoms and having a halogen substituent, an acyl group having 2-8 carbon atoms, an alkenyl group having 2-8 carbon atoms, or an aralkyl group having an aryl moiety of 6-10 carbon atoms and an alkylene moiety of 1-8 carbon atoms,
Q¹ represents hydrogen, halogen, hydroxyl, nitro, amino, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, or an aralkyl group having an aryl moiety of 6-10 carbon atoms and an alkylene moiety of 1-8 carbon atoms,
r is an integer of 1 to 4, and
s is an integer of 1 to 5;
or formula (XX) or a pharmacologically acceptable salt thereof: 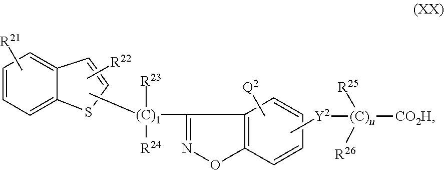
wherein:
R²¹ represents hydrogen, halogen, hydroxyl, nitro, amino, cyano, carboxyl, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, a 5- or 6-membered heterocyclic group, an aralkyl group having an aryl moiety of 6-10 carbon atoms and an alkylene moiety of 1-8 carbon atoms, or an alkyl group having 1-8 carbon atoms and a 5- or 6-membered heterocyclic substituent;
R²² represents hydrogen, an alkyl group having 1-8 carbon atoms, an alkenyl group having 2-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and having an alkoxy substituent having 1-8 carbon atoms, an acyl group having 2-8 carbon atoms, an aryl group having 6-10 carbon atoms, or an aralkyl group having an aryl moiety of 6-10 carbon atoms and an alkylene moiety of 1-8 carbon atoms;
each of R²³, R²⁴, R²⁵ and R²⁶ independently represents hydrogen, an alkyl group having 1-8 carbon atoms, or an alkyl group having 1-8 carbon atoms and having a halogen substituent;
Y² is oxygen, sulfur, NR²⁸ or a bond, R²⁸ representing hydrogen, an alkyl group having 1-8 carbon atoms, an alkyl group having 1-8 carbon atoms and having a halogen substituent, an acyl group having 2-8 carbon atoms, or an alkenyl group having 2-8 carbon atoms;
Q² represents hydrogen, halogen, hydroxyl, nitro, amino, an alkyl group having 1-8 carbon atoms, a 3- to 7-membered cycloalkyl group, an alkenyl group having 2-8 carbon atoms, an alkynyl group having 2-8 carbon atoms, an alkoxy group having 1-8 carbon atoms, an alkyl group having 1-8 carbon atoms and having a 3- to 7-membered cycloalkyl substituent, an alkyl group having 1-8 carbon atoms and having a halogen substituent, an alkyl group having 1-8 carbon atoms and an alkoxy substituent having 1-8 carbon atoms, an alkoxy group having 1-8 carbon atoms and having a halogen substituent, an acyl group having 2-8 carbon atoms, an aryl group having 6-10 carbon atoms, or an aralkyl group having an aryl moiety of 6-10 carbon atoms and an alkylene moiety of 1-8 carbon atoms;
t is an integer of 1 to 4; and
u is an integer of 1 to 5.

2. A method for treating inflammatory conditions in a subject, wherein an inflammatory response is present, and wherein an inflammatory condition is selected from the group consisting of an inflammatory vascular disease, such as atherosclerosis, coronary or peripheral vascular disease, myocardial infarction and stroke; an inflammatory bowel disease, such as Crohn's disease and ulcerative colitis; a systemic inflammatory disorder, such as Lupus Erythematosus; an inflammatory rheumatic disorder, such as rheumatoid arthritis and psoriatic joint disease; and an inflammatory disease of the lung, comprising administering to the subject in need thereof the compound of any one of formulae (I) to (XX) of claim 1 or a pharmaceutically acceptable salt thereof.

3. A method for treating disorders or manifestations of insulin and glucose metabolism, protection of pancreatic beta cells and prevention of microvascular and macrovascular disorders in a subject, wherein the disorders or manifestations of insulin and glucose metabolism are selected form the group consisting of insulin resistance, diabetes, the metabolic syndrome, hypoglycemia, high blood pressure, obesity, and dyslipidemia, comprising administering to the subject in need thereof the compound of any one of formulae (I) to (XX) of claim 1 or a pharmaceutically acceptable salt thereof.

4. A method for treating central or abdominal or visceral obesity in a subject, in which weight loss is required or desired, comprising administering to the subject in need thereof the compound of any one of formulae (I) to (XX) of claim 1 or a pharmaceutically acceptable salt thereof.

5. A method for treating disorders of the skeletal muscle, mitochondrial disease and myopathy in a subject, wherein the mitochondrial disease is selected form the group consisting of myoclonus twiching, epilepsy, ragged red fibers (RRF), hearing loss, exercise intolerance, dementia, and lactic acidosis, comprising administering to the subject in need thereof the compound of any one of formulae (I) to (XX) of claim 1 or a pharmaceutically acceptable salt thereof.

6. A method for treating renal ischemia in a subject, comprising administering to the subject in need thereof the compound of any one of formulae (I) to (XX) of claim 1 or a pharmaceutically acceptable salt thereof.

7. A method for treating myopathy in a subject, comprising administering to the subject in need thereof the compound of any one of formulae (I) to (XX) of claim 1 or a pharmaceutically acceptable salt thereof.

8. A method for treating mitochondrial diseases, Leber's hereditary optic neuropathy (LHON), visual loss beginning in young adulthood, Wolff-Parkinson-White syndrome, multiple sclerosis-type disease, Leigh syndrome, subacute sclerosing encephalopathy, neuropathy, ataxia, retinitis pigmentosa, ptosis (NARP), and myoneurogenic gastrointestinal encephalopathy (MNGIE) in a subject, wherein the mitochondrial diseases are selected from the group consisting of mitochondrial myopathies, diabetes mellitus and deafness (DAD), comprising administering to the subject in need thereof the compound of any one of formulae (I) to (XX) of claim 1 or a pharmaceutically acceptable salt thereof.

9. A method for treating hair loss in a subject comprising administering to the subject in need thereof the compound of any one of formulae (I) to (XX) of claim 1 or a pharmaceutically acceptable salt thereof.

10. A method for wound healing in a subject, comprising administering to the subject in need thereof the compound of any one of formulae (I) to (XX) of claim 1 or a pharmaceutically acceptable salt thereof.

11. The method of any one of claims 1-10, wherein said compound is a PPARδ agonist and is >500 fold selective for PPARδ over PPARα or PPARγ.

12. The method of any one of claims 1-11, wherein said compound is a PPARδ agonist and is >1000 fold selective for PPARδ over PPARα or PPARγ.

13. The method of any one of claims 1-12, wherein the compound is selected from: 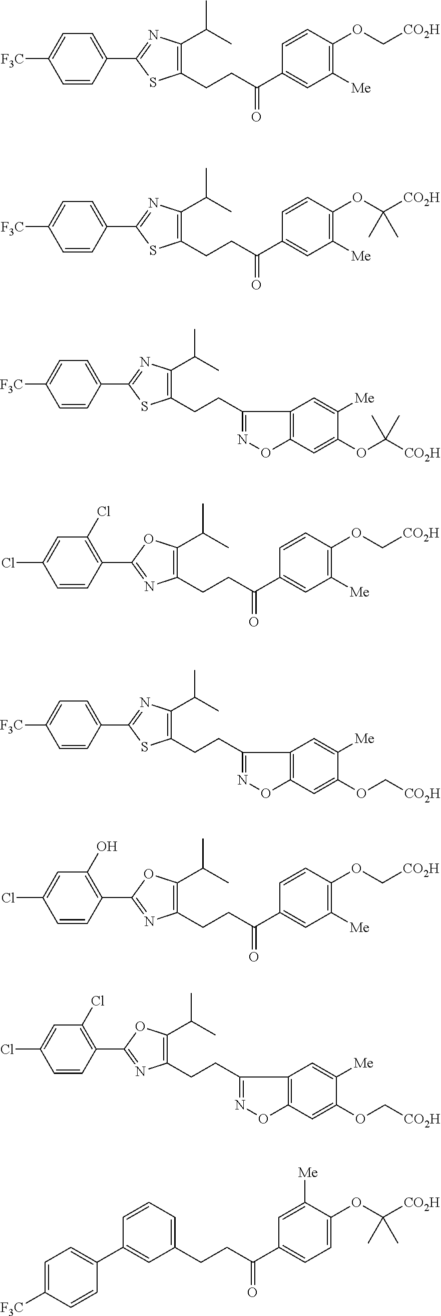 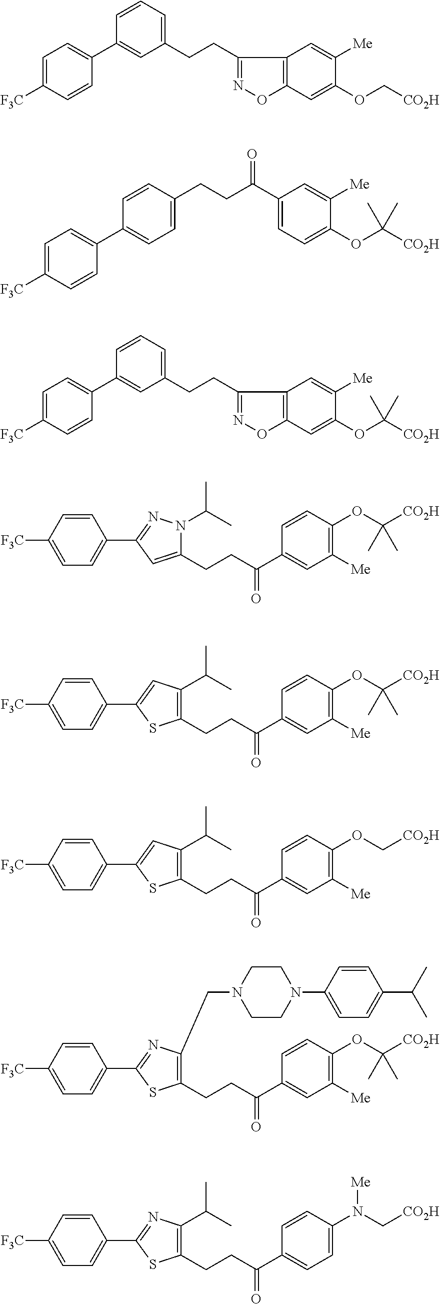 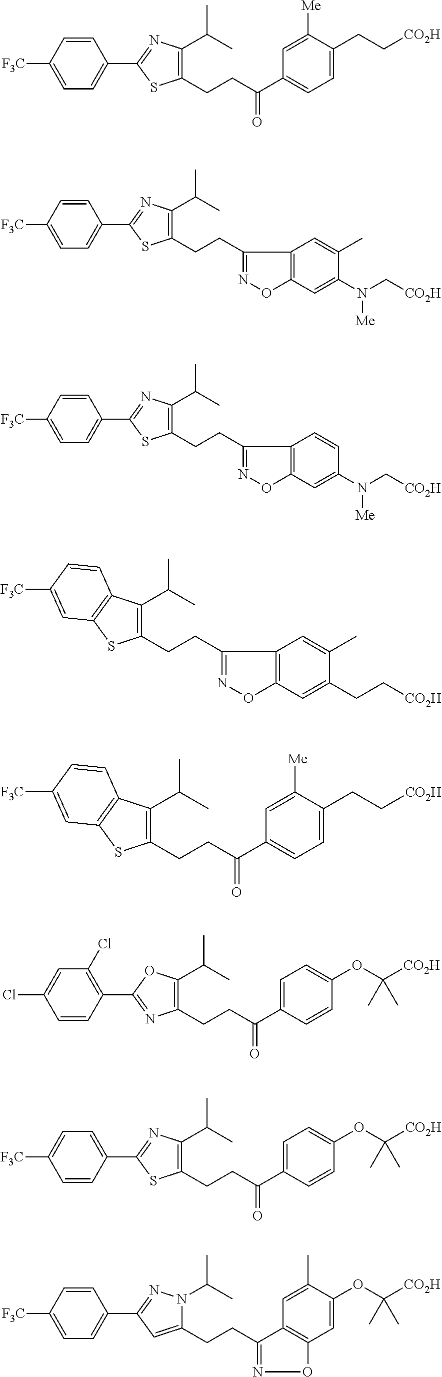 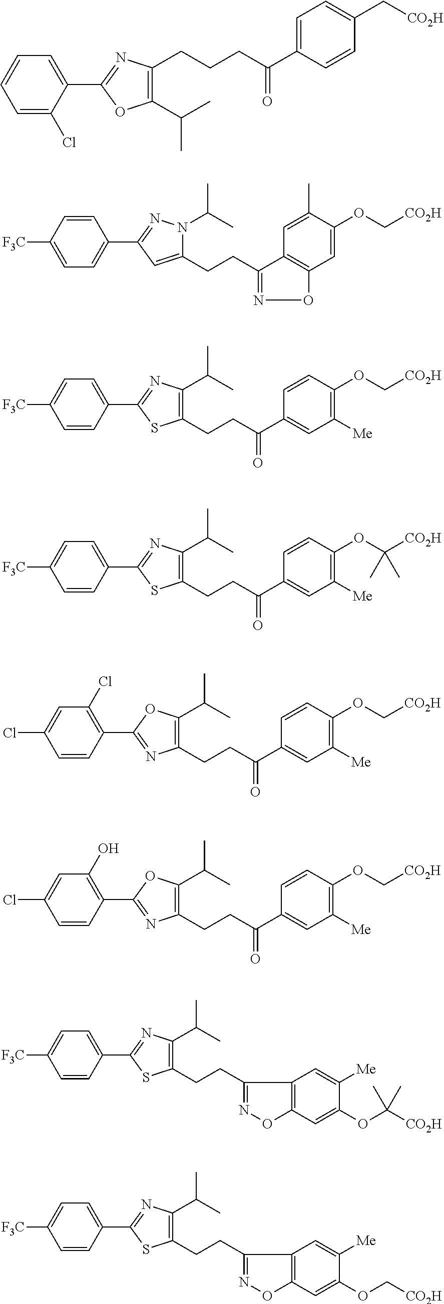 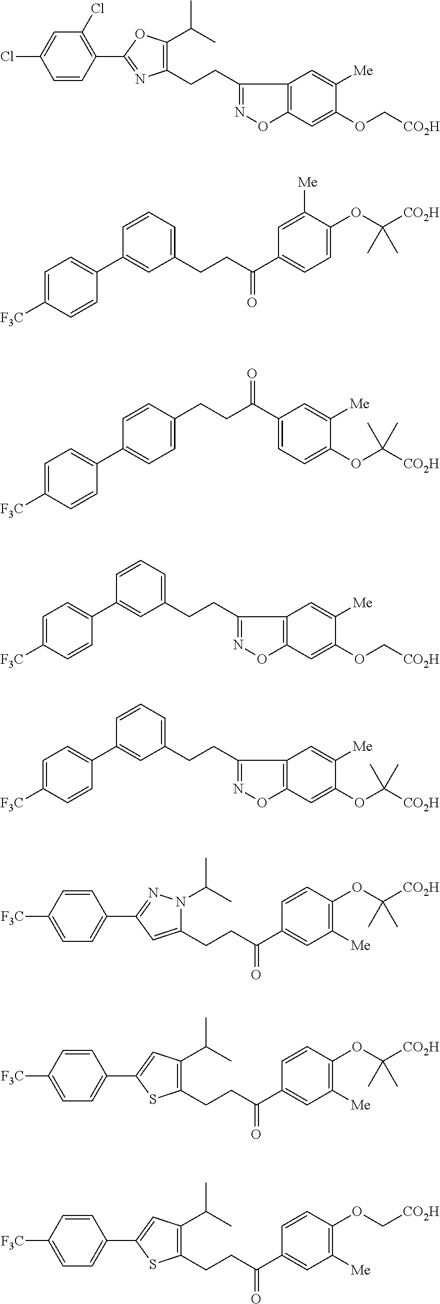 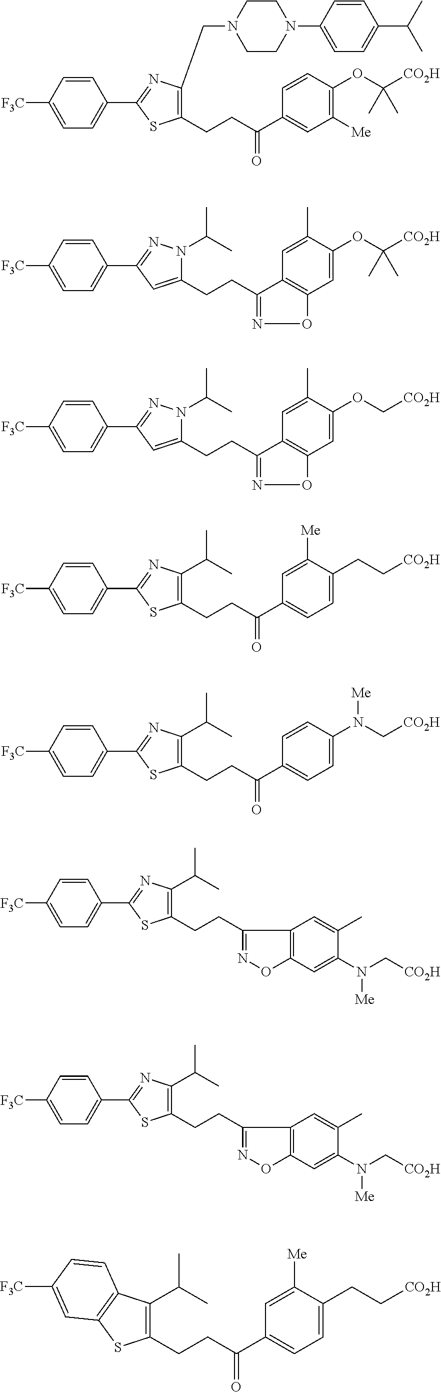 [chemical structure]


SEQUENCE LISTING (4 sequences)

SEQ ID NO: 1 - DNA, 24 bp, Artificial Sequence
  GAL4 binding sequence UASg
cgacggagtactgtcctccgagct

SEQ ID NO: 2 - DNA, 320 bp, Homo sapiens
cgacggagtactgtcctccgagctcgacggagtactgtcctccgagctcgacggagtactgtcctccgagctcgacggagtactgtcctccgagcttctagaggatccggccccgcccagcgtcttgtcattggcgaattcgaacacgcagatgcagtcggggcggcgcggtccgaggtccacttcgcatattaaggtgacgcgtgtggcctcgaacaccgagcgaccctgcagcgacccgcttaacagcgtcaacagcgtgccgcagatctcgaggagcttggcattccggtactgttggtaaaatggaagacgccaaa

SEQ ID NO: 3 - DNA, 683 bp, Homo sapiens
gcttagcgcaacacactgctatgtcagttgacagaaactggaaacaatgatgagagaaggctactactacagcgtgaataagatacgacagagttacaatctccgtatagtcagaggtgacttcggttagatagacactgccgtagaaataagtgtaatagaacatgtttattaggacaattgttacgaaaatataggacatttcttaggtaaaagttttagtacagttccagaagagctcctttttagtcatctttatcgacaaggtcagaaagatcggaactaaggtgaagacagtctacacgggatcagtcgcctctggaaaaccaaaaccctctcatcgctgtgagggtcaacaagaagtctgtgaaccgcgtgaagccaaaaagaaacctcgtgaactcgaaaaattcagccgtttatagcgtacgaacaagctatcttctgtcatcgaaaccgacgacgcgtaacgaatgactcggaaaaccttttagttggttttcagaagcgacgaacctcagactaactcttcgctgtcggtcactcccacttctgcgtctttggaagtgtcatcgaggaggagaatcccaaaatatcttcaggtagtgtagaggggagaggactcgttcgtgtgacgaccccaaaagaagagatggtcctcaattactaagaaacctcaggta

SEQ ID NO: 4 - DNA, 13 bp, Artificial Sequence
  Acyl-CoA oxidase peroxisome proliferator response element sequence
aggacaaaggtca